(19) United States
(12) Patent Application Publication
(10) Pub. No.: US 2023/0093131 A1
(43) Pub. Date: Mar. 23, 2023

(54) ASSESSING AND TREATING ALCOHOL-ASSOCIATED LIVER DISEASE

(71) Applicant: Mayo Foundation for Medical Education and Research, Rochester, MN (US)

(72) Inventors: Harmeet Malhi, Rochester, MN (US); Vijay H. Shah, Rochester, MN (US); Gregory J. Gores, Rochester, MN (US); Tejasav Sehrawat, Rochester, MN (US); Juan Pablo Arab Verdugo, Santiago (CL)

(21) Appl. No.: 17/802,604

(22) PCT Filed: Mar. 12, 2021
(86) PCT No.: PCT/US2021/022096

§ 371 Date: Aug. 26, 2022

Related U.S. Application Data
(60) Provisional application No. 62/989,421, filed on Mar. 13, 2020.

(51) Int. Cl. G01N 33/92

(52) U.S. Cl. CPC G01N 33/92; G01N 2405/08; G01N 2800/085; G01N 2800/52

(57) ABSTRACT

This document relates to methods and materials for assessing and/or treating alcohol-associated liver disease (ALD). For example, methods and materials to determine if a mammal has an ALD are provided herein. This document also relates to materials and methods for using one or more ALD treatments to treat a mammal (e.g., a human) identified as having an ALD.

### CROSS-REFERENCE TO RELATED APPLICATIONS

This application claims the benefit of U.S. Patent Application Ser. No. 62/989,421, filed on Mar. 13, 2020. The disclosure of the prior application is considered part of (and is incorporated by reference in) the disclosure of this application.

### STATEMENT REGARDING FEDERAL FUNDING

This invention was made with government support under AA021788, AA021171, and DK111378 awarded by the National Institutes of Health. The government has certain rights in the invention.

### BACKGROUND

### 1. Technical Field

This document relates to methods and materials for assessing and/or treating liver diseases (e.g., alcohol-associated liver disease (ALD)). For example, methods and materials provided herein can be used to determine if a mammal has an ALD. This document also provides materials and methods for using one or more ALD treatments to treat a mammal (e.g., a human) identified as having an ALD.

### 2. Background Information

Alcohol-associated liver disease (ALD) has been recognized as the most common cause of advanced liver disease worldwide and accounts for 50% of deaths due to cirrhosis (Paula et al., Am J Gastroenterol; 105:1782-1787 (2010); Thursz et al., J Hepatol; 70:521-530 (2019); and Griswold et al., The Lancet; 392:1015-1035 (2018)). Alcoholic hepatitis (AH) is the most severe form of ALD with an extensive inflammatory response and a 30-day mortality rate of around 20-40% (Seitz et al., Nat Rev Dis Primers; 4:16 (2018)). Though health care utilization is high, no pharmacological therapy is associated with improved survival beyond one month (May et al., BMC Gastroenterol; 16(1):129 (2016)). Well-founded circulating biomarkers based on AH pathogenesis that permit diagnosis, inform clinical course, and predict therapeutic targets are lacking.

### SUMMARY

The diagnosis of liver diseases such as ALD remains predominantly clinico-pathological. Biochemical tests are not disease-specific and widespread use of liver biopsies is limited due to risks and poor patient acceptance, and because it is impractical to follow-up response to treatment with serial liver biopsies. Owing to the limitations of these tests, there is an unmet need to identify reproducible, efficient, and non-invasive biomarkers (see, e.g., Singal et al., C/in Gastroenterol Hepatol; 12:555-564 (2014)).

This document provides methods and materials related to assessing and/or treating a liver disease (e.g., an ALD such as AH). In some cases, this document provides methods and materials for identifying a mammal as having an ALD. For example, this document provides methods and materials for detecting the presence or absence of an elevated level of extracellular vesicles (EVs; e.g., circulating EVs) within a sample obtained from a mammal (e.g., a human) and classifying the mammal as having an ALD, based at least in part, on the presence of an elevated level of EVs. In some cases, the presence of an elevated level of EVs (e.g., circulating EVs) in a sample obtained from a mammal (e.g., a human) also can be used to classify the mammal as having an ALD and having an increased risk of mortality (e.g., 90-day mortality). In some cases, this document provides methods and materials for detecting the presence or absence of one or more cargos within EVs (e.g., circulating EVs) in a sample obtained from a mammal (e.g., a human) and classifying the mammal as having an ALD, based at least in part, on the presence, absence, or level of one or more cargos present in the EVs. In some cases, this document provides methods and materials for treating a mammal having an ALD. For example, this document provides methods and materials for using one or more IL-22 polypeptides, one or more inhibitors of a bromodomain-containing protein 4 (BRD4) polypeptide, and/or one or more ALD treatments to treat a mammal in need thereof (e.g., a mammal identified as having an ALD as described herein).

As demonstrated herein, an elevated level of circulating EVs and/or the presence of particular cargos within circulating EVs can be present in plasma obtained from an AH patient. For example, humans having AH can exhibit circulating EVs of greater than about 1.5×10¹¹ EVs per milliliter (EVs/mL) of plasma (e.g., a level that was elevated as compared with heavy drinkers, healthy controls, and end-stage liver disease due to NASH or chronic cholestatic diseases). In some cases, humans having AH and having circulating EVs of greater than about 5×10¹¹ EVs/mL plasma can experience a high risk of 90-day mortality and can be classified as having a high risk of 90-day mortality. For example, humans having AH can exhibit the presence of enriched cargos (e.g., enriched sphingosine (SPH), enriched sphinganine (SPA), enriched sphingosine 1-phosphate (S1P), enriched C14:0 ceramides (C14-cer), enriched C16:0 ceramides (C16-cer), enriched C18:0 ceramides (C18-cer), enriched C20:0 ceramides (C20-cer), enriched C22:0 ceramides (C22-cer), enriched C24:0 ceramides (C24-cer), and/or enriched C24:1 ceramides (C24-cer) cargos)) in circulating EVs (e.g., as compared with healthy controls and heavy drinkers).

Having the ability to identify a mammal as having an ALD based, at least in part, on an elevated level of circulating EVs and/or the presence of particular cargos within circulating EVs provides a unique and unrealized opportunity to identify a mammal (e.g., a human) as having an ALD, as opposed to a mammal that is a heavy alcohol drinker or a mammal having other etiologies of liver disease, using a liquid biopsy (e.g., without the need for a liver biopsy). In addition, the ability to assess 90-day survival of a patient based, at least in part, on an elevated level of circulating EVs and/or the presence of particular cargos within circulating EVs can be used to select a targeted approach for treating that patient.

In general, one aspect of this document features methods for treating a mammal having an ALD. The methods can include, or consist essentially of, identifying a mammal as having greater than 1.5×10¹¹ circulating EVs per mL in a sample obtained from the mammal, and administering an inhibitor of a BRD4 polypeptide to the mammal. The mammal can be a human. The sample can be a blood sample (e.g., a plasma sample). The ALD can be alcoholic hepatitis. The circulating EVs can include an enriched sphingolipid cargo. The enriched sphingolipid cargo can be sphinganine (SPA), sphingosine (SPH), sphingosine-1-phosphate (S1P), a 14:0 ceramide, a C16:0 ceramide, a C18:0 ceramide, a C20:0 ceramide, a C22:0 ceramide, a C24:1 ceramide, a C24:0 ceramide, or combinations thereof. The enriched sphingolipid cargo can be SPA, and the circulating EVs can include from about 0.9 nM to about 34.02 nM of the SPA. The enriched sphingolipid cargo can be 14:0 ceramide, and the circulating EVs can include from about 0.25 nM to about 54.7 nM of the 14:0 ceramide. The enriched sphingolipid cargo can be C16:0 ceramide, and the circulating EVs can include from about 19 nM to about 1765.9 nM of the C16:0 ceramide. The enriched sphingolipid cargo can be C24:0 ceramide, and the circulating EVs can include from about 23.2 nM to about 2030.62 nM of the C24:0 ceramide. The inhibitor can be iBET151, RVX-208, iBET 762, Zen-3694, JQ1, OTX-015, GSK620, or ABBV-744.

In another aspect, this document features methods for treating a mammal having an ALD. The methods can include, or consist essentially of, administering an inhibitor of a BRD4 polypeptide to a mammal identified as having greater than 1.5×10¹¹ circulating EVs per mL in a sample obtained from the mammal. The mammal can be a human. The sample can be a blood sample (e.g., a plasma sample). The ALD can be alcoholic hepatitis. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or combinations thereof. The enriched sphingolipid cargo can be SPA, and the circulating EVs can include from about 0.9 nM to about 34.02 nM of the SPA. The enriched sphingolipid cargo can be 14:0 ceramide, and the circulating EVs can include from about 0.25 nM to about 54.7 nM of the 14:0 ceramide. The enriched sphingolipid cargo can be C16:0 ceramide, and the circulating EVs can include from about 19 nM to about 1765.9 nM of the C16:0 ceramide. The enriched sphingolipid cargo can be C24:0 ceramide, and the circulating EVs can include from about 23.2 nM to about 2030.62 nM of the C24:0 ceramide. The inhibitor can be iBET151, RVX-208, iBET 762, Zen-3694, JQ1, OTX-015, GSK620, or ABBV-744.

In another aspect, this document features methods for treating a mammal having an ALD. The methods can include, or consist essentially of, identifying a mammal as having greater than 5×10¹¹ circulating EVs per mL in a sample obtained from the mammal, and administering an inhibitor of a BRD4 polypeptide to the mammal. The mammal can be a human. The sample can be a blood sample (e.g., a plasma sample). The ALD can be alcoholic hepatitis. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or combinations thereof. The enriched sphingolipid cargo can be SPA, and the circulating EVs can include from about 0.9 nM to about 34.02 nM of the SPA. The enriched sphingolipid cargo can be 14:0 ceramide, and the circulating EVs can include from about 0.25 nM to about 54.7 nM of the 14:0 ceramide. The enriched sphingolipid cargo can be C16:0 ceramide, and the circulating EVs can include from about 19 nM to about 1765.9 nM of the C16:0 ceramide. The enriched sphingolipid cargo can be C24:0 ceramide, and the circulating EVs can include from about 23.2 nM to about 2030.62 nM of the C24:0 ceramide. The inhibitor can be iBET151, RVX-208, iBET 762, Zen-3694, JQ1, OTX-015, GSK620, or ABBV-744.

In another aspect, this document features methods for treating a mammal having an ALD. The methods can include, or consist essentially of, administering an inhibitor of a BRD4 polypeptide to a mammal identified as having greater than 5×10¹¹ circulating EVs per mL in a sample obtained from the mammal. The mammal can be a human. The sample can be a blood sample (e.g., a plasma sample). The ALD can be alcoholic hepatitis. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or combinations thereof. The enriched sphingolipid cargo can be SPA, and the circulating EVs can include from about 0.9 nM to about 34.02 nM of the SPA. The enriched sphingolipid cargo can be 14:0 ceramide, and the circulating EVs can include from about 0.25 nM to about 54.7 nM of the 14:0 ceramide. The enriched sphingolipid cargo can be C16:0 ceramide, and the circulating EVs can include from about 19 nM to about 1765.9 nM of the C16:0 ceramide. The enriched sphingolipid cargo can be C24:0 ceramide, and the circulating EVs can include from about 23.2 nM to about 2030.62 nM of the C24:0 ceramide. The inhibitor can be iBET151, RVX-208, iBET 762, Zen-3694, JQ1, OTX-015, GSK620, or ABBV-744.

In another aspect, this document features methods for treating a mammal having an ALD. The methods can include, or consist essentially of, identifying a mammal as having circulating EVs comprising enriched sphingolipid cargo in a sample obtained from the mammal, and administering an inhibitor of a BRD4 polypeptide to the mammal. The mammal can be a human. The sample can be a blood sample (e.g., a plasma sample). The ALD can be alcoholic hepatitis. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or combinations thereof. The enriched sphingolipid cargo can be SPA, and the circulating EVs can include from about 0.9 nM to about 34.02 nM of the SPA. The enriched sphingolipid cargo can be 14:0 ceramide, and the circulating EVs can include from about 0.25 nM to about 54.7 nM of the 14:0 ceramide. The enriched sphingolipid cargo can be C16:0 ceramide, and the circulating EVs can include from about 19 nM to about 1765.9 nM of the C16:0 ceramide. The enriched sphingolipid cargo can be C24:0 ceramide, and the circulating EVs can include from about 23.2 nM to about 2030.62 nM of the C24:0 ceramide. The inhibitor can be iBET151, RVX-208, iBET 762, Zen-3694, JQ1, OTX-015, GSK620, or ABBV-744.

In another aspect, this document features methods for treating a mammal having an ALD. The methods can include, or consist essentially of, administering an inhibitor of a BRD4 polypeptide to a mammal identified as having circulating EVs comprising enriched sphingolipid cargo in a sample obtained from the mammal. The mammal can be a human. The sample can be a blood sample (e.g., a plasma sample). The ALD can be alcoholic hepatitis. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or combinations thereof. The enriched sphingolipid cargo can be SPA, and the circulating EVs can include from about 0.9 nM to about 34.02 nM of the SPA. The enriched sphingolipid cargo can be 14:0 ceramide, and the circulating EVs can include from about 0.25 nM to about 54.7 nM of the 14:0 ceramide. The enriched sphingolipid cargo can be C16:0 ceramide, and the circulating EVs can include from about 19 nM to about 1765.9 nM of the C16:0 ceramide. The enriched sphingolipid cargo can be C24:0 ceramide, and the circulating EVs can include from about 23.2 nM to about 2030.62 nM of the C24:0 ceramide. The inhibitor can be iBET151, RVX-208, iBET 762, Zen-3694, JQ1, OTX-015, GSK620, or ABBV-744.

In another aspect, this document features methods for treating a mammal having an ALD. The methods can include, or consist essentially of, identifying a mammal as having greater than 1.5×10¹¹ circulating EVs per mL in a sample obtained from the mammal; and administering an IL-22 polypeptide or an ALD treatment to the mammal. 5 The mammal can be a human. The sample can be a blood sample (e.g., a plasma sample). The ALD can be AH. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be 10 SPA, and the circulating EVs can include from about 0.9 nM to about 34.02 nM of SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide. The can include administering the ALD treatment to the mammal, and the ALD treatment can be administering nutritional supplementation, alcohol cessation counseling, administering corticosteroids, administering pentoxifylline, or any combinations thereof.

In another aspect, this document features methods for treating a mammal having an ALD. The methods can include, or consist essentially of, administering an IL-22 polypeptide or an ALD treatment to a mammal identified as having greater than 1.5×10¹¹ circulating EVs per mL in a sample obtained from the mammal. The mammal can be a human. The sample can be a blood sample (e.g., a plasma sample). The ALD can be AH. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be SPA, and the circulating EVs can include from about 0.9 nM to about 34.02 nM of SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide. The can include administering the ALD treatment to the mammal, and the ALD treatment can be administering nutritional supplementation, alcohol cessation counseling, administering corticosteroids, administering pentoxifylline, or any combinations thereof.

In another aspect, this document features methods for treating a mammal having an ALD and as being at high risk of mortality. The methods can include, or consist essentially of, identifying a mammal as having greater than 5×10¹¹ circulating EVs per mL in a sample obtained from the mammal; and administering an IL-22 polypeptide or an ALD treatment to the mammal. The mammal can be a human. The sample can be a blood sample (e.g., a plasma sample). The ALD can be AH. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be SPA, and the circulating EVs can include from about 0.9 nM to about 34.02 nM of SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide. The method can include administering the ALD treatment to the mammal, and the ALD treatment can be administering corticosteroids, administering pentoxifylline, and/or undergoing a liver transplantation.

In another aspect, this document features methods for treating a mammal having an ALD and as being at high risk of mortality. The methods can include, or consist essentially of, administering an IL-22 polypeptide or an ALD treatment to a mammal identified as having greater than 5×10¹¹ circulating EVs per mL in a sample obtained from the mammal. The mammal can be a human. The sample can be a blood sample (e.g., a plasma sample). The ALD can be AH. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be SPA, and the circulating EVs can include from about 0.9 nM to about 34.02 nM of SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide. The method can include administering the ALD treatment to the mammal, and the ALD treatment can be administering corticosteroids, administering pentoxifylline, and/or undergoing a liver transplantation.

In another aspect, this document features methods for treating a mammal having an ALD. The methods can include, or consist essentially of, identifying a mammal as having circulating EVs including enriched sphingolipid cargo in a sample obtained from the mammal; and administering an IL-22 polypeptide or an ALD treatment to the mammal. The mammal can be a human. The sample can be a blood sample (e.g., a plasma sample). The ALD can be AH. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be SPA, and the circulating EVs can include from about 0.9 nM to about 34.02 nM of SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide. The method can include administering the ALD treatment to the mammal, and the ALD treatment can be administering nutritional supplementation, alcohol cessation counseling, administering corticosteroids, administering pentoxifylline, or any combinations thereof.

In another aspect, this document features methods for treating a mammal having an ALD. The methods can include, or consist essentially of, administering an IL-22 polypeptide or an ALD treatment to a mammal identified as having circulating EVs including enriched sphingolipid cargo in a sample obtained from a mammal. The mammal can be a human. The sample can be a blood sample (e.g., a plasma sample). The ALD can be AH. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be SPA, and the circulating EVs can include from about 0.9 nM to about 34.02 nM of SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide. The method can include administering the ALD treatment to the mammal, and the ALD treatment can be administering nutritional supplementation, alcohol cessation counseling, administering corticosteroids, administering pentoxifylline, or any combinations thereof.

In another aspect, this document features methods for identifying a mammal having an ALD. The methods can include, or consist essentially of, detecting the presence of greater than 1.5×10¹¹ circulating EVs per mL in a sample obtained from a mammal; and classifying the mammal as having ALD. The mammal can be a human. The sample can be a blood sample (e.g., a plasma sample). The ALD can be AH. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be SPA, and the circulating EVs can include from about 0.9 nM to about 34.02 nM of SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide.

In another aspect, this document features methods for identifying a mammal having an ALD and as being at high risk of mortality. The methods can include, or consist essentially of, detecting the presence of greater than 5×10¹¹ circulating EVs per mL in a sample obtained from a mammal; and classifying the mammal as having ALD and as being at high risk of mortality. The mammal can be a human. The sample can be a blood sample (e.g., a plasma sample). The ALD can be AH. The circulating EVs can include an enriched sphingolipid cargo. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be SPA, and the circulating EVs can include from about 0.9 nM to about 34.02 nM of said SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide.

In another aspect, this document features methods for identifying a mammal having an ALD. The methods can include, or consist essentially of, detecting the presence of circulating EVs including enriched sphingolipid cargo in a sample obtained from a mammal; and classifying the mammal as having ALD. The mammal can be a human. The sample can be a blood sample (e.g., a plasma sample). The ALD can be AH. The enriched sphingolipid cargo can be SPA, SPH, S1P, a 14:0 ceramide, a C16:0 ceramide, a C18:0 ceramide, a C20:0 ceramide, a C22:0 ceramide, a C24:1 ceramide, a C24:0 ceramide, or any combinations thereof. The enriched sphingolipid cargo can be SPA, and the circulating EVs can include from about 0.9 nM to about 34.02 nM of SPA. The enriched sphingolipid cargo can be 14:0 ceramide, and the circulating EVs can include from about 0.25 nM to about 54.7 nM of 14:0 ceramide. The enriched sphingolipid cargo can be C16:0 ceramide, and the circulating EVs can include from about 19 nM to about 1765.9 nM of C16:0 ceramide. The enriched sphingolipid cargo can be C24:0 ceramide, and the circulating EVs can include from about 23.2 nM to about 2030.62 nM of C24:0 ceramide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1: EVs are elevated in plasma from AH subjects and correlate with disease severity. (A) EV counts were significantly elevated in AH when compared to healthy controls (p<0.0001), heavy drinkers (p<0.0001), and decompensated alcoholic cirrhosis subjects (p<0.0001). Heavy drinkers had significantly higher EV counts than healthy controls (p=0.0068). (B) EV counts were significantly higher in AH when compared to ESLD (p<0.0001). MELD scores were comparable between NASH, cholestatic liver diseases, and AH subgroups. (C) EV counts were significantly higher in severe AH subjects (MELD>20) when compared with mild/moderate AH subjects. (D) EV counts were also significantly correlated with MELD and CTP scores (p<0.0001).

FIG. 2: Isolated nanoparticles were characterized as small EVs. (A) Nanoparticle size distribution for all groups was within the 50-150 nm small EV range. (B) Immunoblotting was done for EV markers CD81 and TSG101. EVs used for each sample were isolated from identical volumes of plasma. (C) Immunoblotting was done for VLDL/LDL component ApoB100 and HDL component ApoA1. EVs used for each sample were isolated from identical volumes of plasma. Amount of EVs used is the same as our targeted sphingolipids measurements experiment. The final band represents 1:20 dilution of plasma from an AH subject. AH=alcoholic hepatitis, L=protein ladder and P=plasma.

FIG. 3: EV concentration has high performance in diagnosing AH. (A) High diagnostic accuracy was noted for diagnosis of AH versus healthy controls in the discovery cohort. A cut-off value of 1.56×10¹¹ particles/mL was determined using Youden's index. (B) In the complete cohort AH vs. MELD matched severe end-stage liver disease and decompensated alcoholic cirrhosis subjects was diagnosed with high sensitivity and specificity using 1.56×10¹¹ particles/mL. (C) The diagnostic performance in our complete cohort of AH subjects was high compared to MELD-matched severe end-stage liver disease subjects with NASH or cholestatic liver diseases. (D) The diagnostic performance in our complete cohort of AH subjects was high compared to decompensated alcoholic cirrhosis subjects.

FIG. 4: EV concentration has high performance in diagnosing AH. (A) High diagnostic accuracy was noted for diagnosis of AH versus healthy controls in the discovery cohort. A cut-off value of 1.5×10¹¹ particles/mL was determined using Youden's index. (B) In the validation cohort AH vs. healthy controls was diagnosed with high sensitivity and specificity using 1.5×10¹¹ particles/mL. (C) The diagnostic performance in our complete cohort of AH subjects was high compared to healthy controls and (D) heavy drinkers.

FIG. 5: EV counts have utility in predicting 90-day survival. (A) AH subjects were dichotomized at the median and divided into two subgroups of high and low EV counts. (B) Patients in the high EV count group had significantly higher 90-day mortality compared to patients in the low EV count group (logrank p=0.015).

FIG. 6: EV cargo is enriched in sphingolipid species in AH subjects. (A) Heatmap and hierarchical clustering depicts differences in concentration of sphingolipid species in healthy controls, heavy drinkers, alcoholic cirrhosis, ESLD, and alcoholic hepatitis subjects. EV sphingolipid cargo from healthy controls and ESLD subjects clusters together and cargo from alcoholic cirrhosis and heavy drinkers cluster together. EVs from AH subjects are highly-enriched in multiple sphingolipid species and cluster separately. (B) 3D principal component analysis (PCA) graphs demonstrate differential clustering of sphingolipid species in EVs and plasma from healthy controls (light grey) and AH subjects (black). (C) 3D and 2D PCA graphs help demonstrate differential clustering of sphingolipid species in EV cargo from healthy controls (light grey), cirrhotics including alcoholic, NASH, PBC, and PSC subjects (grey), and AH subjects (black). (D) Multiple sphingolipid species, especially long chain ceramides, were significantly raised in AH compared with healthy controls, heavy drinkers, decompensated AC and ESLD subjects. These values are shown here as fold-change over controls. (E) In contrast, only C16:0 ceramide and C24:1 ceramide showed significantly increased concentration in AH. p-values of ≤0.05, 0.01, 0.001, and 0.0001 were denoted as *, **, #, and ##respectively.

FIG. 7: EV x sphingolipid cargo concentrations correlate positively with MELD score. Correlation between EV concentration and all individual sphingolipids were assessed using linear regression modeling and multiple entities showed significant positive correlation with MELD score. These included log EV by itself, as well as SPH, SPA, S1P, C14:0 ceramide, C16:0 ceramide, C18:0 ceramide, C18:1 ceramide, C20:0 ceramide and C24:1 ceramide.

FIG. 8: EV x sphingolipid cargo concentrations correlate positively with CTP score. Correlation between EV concentration and all individual sphingolipids were assessed using linear regression modeling and multiple entities showed significant positive correlation with CTP score. These included log EV by itself, as well as SPH, SPA, S1P, C14:0 ceramide, C16:0 ceramide, C18:0 ceramide, C18:1 ceramide, C20:0 ceramide and C24:1 ceramide.

FIG. 9: EV sphingolipid cargo can predict severity of disease in AH subjects. ROC curves were generated to assess role of EV and individual sphingolipids in predicting disease severity. Log EV (AUC=0.88, p=<0.0001), SPH (AUC=0.85, p<0.0001), SPA (AUC=0.85, p<0.0001), S1P (AUC=0.81, p=0.0002), C14:0 ceramide (AUC=0.82, p=0.0001), C16:0 ceramide (AUC=0.83, p<0.0001), C18:1 ceramide (AUC=0.76, p=0.0009), C18:0 ceramide (AUC=0.74, p=0.001), C20:0 ceramide (AUC=0.73, p=0.001), C22:0 ceramide (AUC=0.74, p=0.001), C24:1 ceramide (AUC=0.80, 0.0001), and C24:0 (AUC=0.72, 0.002) all resulted in significant ROC curves with high AUROC curve values. (SPH=sphingosine, SPA=sphinganine, and S1P=Sphingosine 1-phosphate).

FIG. 10: EV sphingolipid may improve the performance of MELD score. (A) ROC curves were generated to assess the role of EV and individual sphingolipids in predicting 90-day mortality in subjects. Again, the same sphingolipid species had the best performance. Log EV (AUC=0.81, p=0.003), SPH (AUC=0.78, p=0.008), SPA (AUC=0.78, p=0.009), S1P (AUC=0.73, p=0.04), C14:0 ceramide (AUC=0.76, p=0.03), C16:0 ceramide (AUC=0.77, p=0.01), and C24:1 ceramide (AUC=0.75, p=0.03) all resulted in statistically-significant ROC curves. Youden's index was used to assess optimal cut-off to dichotomize the cohort for predicting 90-day mortality, and these were then used to generate KM curves for predicting survival. All 6 EV x sphingolipid species resulted in statistically-significant KM curve graphs (all p<0.01). (B) Log EV count and sphingolipid cargo improved the performance of MELD score in predicting 90-day mortality in our cohort. MELD had an AUC value of 0.86. When log EV was taken into consideration with MELD, the AUC did not improve. However, on addition of the 6 sphingolipid species, which were found to be significantly associated with 90-day mortality in Cox's univariate analysis to MELD and log EV, the AUC value increased to 0.91. (SPH=sphingosine, SPA=sphinganine, and S1P=Sphingosine 1-phosphate).

FIG. 11: EV C16 ceramide and S1P content are increased in AH. C16 ceramide (A) and S1P (B) were quantified by LC-MS/MS in EVs isolated from 800 μL plasma. EV C16 ceramide and S1P were normalized to the number of EVs. Significance was calculated using a two tailed t test for the indicated pairwise comparisons. n=6 each.

FIG. 12: EV C16 ceramide content correlates with liver injury. EV C16 ceramide was correlated with INR (A) Maddreys Discriminant Function (B) and MELD (C) in AH, HD and Healthy Controls. n=6, each.

FIG. 13: EV sphingolipids decrease with AH resolution. The EV C16 ceramide and S1P content correlate with AH resolution over time. n=6 per each group. *p<0.05.

FIG. 14: EV-activated macrophage inflammation is SIP-dependent. A) TNF-α and B) IL-1β responses in primary human macrophages from AH subjects treated with EVs derived from HD (HD EV) or AH (AH EV) for 4 hours showing a significant increase in both cytokines by AH-EVs. The proinflammatory effects of EVs were significantly reduced by S1P receptor inhibitor FTY 720, 1 μM. n=3, *p<0.05, **p<0.01.

FIG. 15: Exemplary experimental schema for mechanism of IL-22-induced AH resolution.

FIG. 16: ASGR is a marker of hepatocyte-derived EV. A) Immuno-gold transmission electron microscopy with omission of primary antibodies (control) and labeling with ASGR2 (15 nm particle, arrowhead) and Cyp2E1 (10 nm particle, arrows). Scale bar=100 nm. B) Western blot demonstrating the specificity of ASGR2 antibody, α-tubulin is loading control.

FIG. 17: Efficient IAMC-capture of hepatocyte-derived EVs. IAMC of hepatocyte-derived EVs with ASGR2 antibody (Pellet). Exosomes were isolated from ASGR2 EV depleted plasma using UTC (FT). ASGR2 antibody captured hepatocyte-derived EVs with high efficiency as demonstrated by significant enrichment of EV markers in the pellet in comparison to FT.

FIG. 18: Bromodomain inhibitors suppresses expression of CXCLs by inhibition of transcription factor binding at CXCL Super Enhancer (SE). Pretreatment of HHSEC with varying amounts of pan-BD inhibitor iBET151 suppresses CXCL expression after TNFα stimulation.

FIG. 19: Bromodomain inhibitor iBET151 attenuates liver CXCL production and neutrophil infiltration in a murine chronic alcohol feeding/LPS model. C57BL6 mice were fed 5% alcohol diet or pair-fed diet for 10 days. A subset of mice also received IP injection of iBET151. Alcohol fed mice also received IP injection of iBET151 and LPS on day 11, 9 and 8 hours before sacrifice respectively (n=4-14). qPCRs demonstrate the multiple of CXCL chemokine production were elevated with alcohol feeding and LPS injection which were attenuated by iBET151. Neutrophil marker Ly6g reflects increased neutrophil liver infiltration with alcohol/LPS which were reduced with iBET151. ALT elevations were also seen with alcohol/LPS mice and attenuated by iBET151.

FIG. 20: Bromodomain inhibitors suppress expression of CXCLs by inhibition of transcription factor binding at CXCL super enhancer and promoter sites. A) LSECs were pretreated with iBET151 (0-50 M) (n=4). CXCL1, 6, and 8 expression levels were assessed by qPCR. Expression levels were normalized to basal condition and log₁₀ fold change values were plotted. One-way matched-pairs ANOVA analysis was performed with Post-hoc Dunnett's multiple comparison correction. B and C) ChIP-qPCR assays for BRD4 binding (B) or NF-κB binding (C) with and without TNFα stimulation was assessed at CXCL super-enhancer and CXCL1 promoter sites after pretreatment with iBET151. D and E) Same experiments were repeated with Celastrol. Enrichment for either CXCL1 promoter (Promoter) or CXCL super enhancer (Super Enhancer) sequence was examined. Sequence enrichment was normalized to input. Box plots show top quartiles and third quartiles; whiskers show maximum and minimum values. Two-way matched-pairs ANOVA was performed on log-transformed input percentage, with Post-hoc Sidak's multiple comparisons correction (n=3).

FIG. 21: Bromodomain inhibitors suppress expression of CXCLs. LSECs were pretreated with iBET151 (0-50 M) (n=4). CXCL2, 3, and 5 expression levels were assessed by qPCR. Expression levels were normalized to basal condition and log₁₀ fold change values were plotted. One-way matched-pairs ANOVA analysis was performed with Post-hoc Dunnett's multiple comparison correction. Error bars indicate SD. There was a linear trend for decreasing CXCL2, 3, 5 expression with increasing iBET151 concentrations with TNFα, and with increasing iBET151 concentrations without TNFα.

FIG. 22: iBET151 LSEC cytotoxicity assay. IncuCyte system was utilized to image cells and assess for cytotoxicity. Dying cells (black arrows) are stained green by fluorescent dye, and green cells are counted for each low power image field. Cells were pretreated with control medium or iBET151 and/or TNFα, and quantification for the percentage of dead cells was done at ˜30 minutes intervals for 10 hours starting at 15 minutes. Images shown were acquired after 4 hours of incubation. There was no difference among various treatment groups. Error bars indicate SD, n=3. Two-way RM ANOVA analysis was performed showing no significant difference among treatment groups.

FIG. 23: Bromodomain Inhibitor iBET151 Attenuates Liver CXCL Production and Neutrophil Infiltration in a Murine Alcohol Feeding/LPS Model. A) Schematic of alcohol feeding protocol. Mice were fed 5% alcohol diet or pair-fed control diet for 10 days. A subset of mice also received IP injection of iBET151 compound. Alcohol fed mice also received IP injection of LPS on day 11, 8 hours before sacrifice (n=6-8 in pair-fed groups, n=12-14 in alcohol fed groups). B) qPCRs demonstrated CXCL chemokine and neutrophil marker Ly6g elevation with alcohol/LPS treatment. This response was attenuated by iBET151 injections. Expression levels were normalized to average expression of pair-fed control mice. C) IHC for MPO (neutrophil marker) is shown, with number of neutrophils per low power fields on y-axis. D) Serum ALT levels were elevated in EtOH/LPS mice compared to pair-fed mice, but no statistical difference was noted with iBET151 administration. For all analysis, two-way ANOVA was performed on normalized expression values for qPCRs or cell counts for IHC staining or ALT values, with Post-hoc Tukey's multiple comparisons correction.

FIG. 24: Comparison of LPS Alone Treatment with Combination Alcohol-feeding/LPS Treatment Mice. A) qPCRs of CXCL chemokine and neutrophil marker Ly6g were normalized to average expression of Pair-fed control mice (n=12). Increased Cxcl1 and Cxcl2 expression was seen in combination group (n=12) compared to LPS alone (n=10). No significant difference was seen with Ly6g. B) BODIPY 493/503 staining of mice liver was performed. Scatter plot shows normalized quantification of BODIPY 493/503 staining to Pair-fed mice (n=12 for Pair-fed, n=14 for EtOH+LPS, and n=10 for LPS only). One-way ANOVA was performed with qPCR fold changes or BODIPY quantification ratios, with Post-hoc Sidak's multiple comparison correction. Error bars indicate SD.

FIG. 25: BODIPY 493/503 Stain for Analysis of Steatosis in Chronic Alcohol Feeding/LPS Injection Mice with iBET151. Frozen section of mouse liver was stained with BODIPY 493/503 (stained green). DAPI was used to stain nuclei (stained blue). Scatter plot shows collective normalized quantification of BODIPY 493/503 staining. There is increased steatosis in alcohol-fed/LPS mice compared to control, and this increase was attenuated with iBET151 administration in the treatment arm (n=12 Pair-fed/Veh, n=14 EtOH+LPS/Veh, n=8 Pair-fed/iBET151, n=6 EtOH+LPS/iBET151). Two-way ANOVA was performed on normalized quantification ratios. Error bars indicate SD.

FIG. 26: Alcohol feeding/LPS Injection Increased Cleaved Caspase 3 in Mice Livers. A) Representative Western Blotting for caspase 3 showed increased cleaved caspase 3 in livers of alcohol-fed/LPS mice. This increase was attenuated by iBET151 injection. There is no difference to total Caspase 3 levels (n=12 Pair-fed/Veh, n=14 EtOH+LPS/Veh, n=8 Pair-fed/iBET151, n=6 EtOH+LPS/iBET151, n=8 LPS only). Quantification of the bands were shown. B and C) Quantification of Malondialdehyde (MDA) assessing lipid peroxidation (B) (n=6 Pair-fed/Veh, n=7 EtOH+LPS/Veh, n=6 Pair-fed/iBET151, n=5 EtOH+LPS/iBET151), and 8-Hydroxy-2′-deoxyguanosine (8-OHdG) assessing DNA oxidation (C) (n=6 Pair-fed/Veh, n=4 EtOH+LPS/Veh, n=4 Pair-fed/iBET151, n=4 EtOH+LPS/iBET151) in mice livers showed no difference among treatment groups. One-way ANOVA analysis was performed with Post-hoc Dunnett's multiple comparison correction. Error bars indicate SD.

### DETAILED DESCRIPTION

This document provides methods and materials related to assessing and/or treating a liver disease (e.g., an ALD such as AH). In some cases, this document provides methods and materials for identifying a mammal as having an ALD. As described herein, an elevated level of EVs (e.g., circulating EVs) can be present in a sample obtained from a mammal (e.g., a human) having ALD. For example, a mammal (e.g., a human) can be identified as having ALD based, at least in part, on the presence of an elevated level of EVs (circulating EVs) in a sample obtained from that mammal. For example, a mammal (e.g., a human) suspected to have ALD can be identified as having ALD based, at least in part, on the presence of an elevated level of EVs (circulating EVs) in a sample obtained from that mammal. Mammals can be suspected as having ALD based, at least in part, on a history of alcohol use (e.g., greater than 40 g/day in females and greater than about 60 g/day in males for a minimum period of at least about 6 months), a bilirubin level of greater than about 3 mg/dL, and/or a level of aspartate transaminase (AST) polypeptides that is greater than 1.5 times a level of alanine transaminase (ALT) polypeptides where both the level of AST polypeptides and the level of ALT polypeptides are less than about 500 U/L. In some cases, a mammal (e.g., a human) suspected to have ALD can be a mammal identified as unlikely to have cardiovascular diseases such as coronary syndromes (e.g., acute coronary syndromes), cancer, and/or a liver disease due to other causes (e.g., nonalcoholic steatohepatitis, primary sclerosing cholangitis, and primary biliary cholangitis). In some cases, a mammal (e.g., a human) identified as having an ALD based, at least in part, on the presence of an elevated level of EVs (e.g., circulating EVs) in a sample obtained from a mammal can be assessed for an increased risk of mortality (e.g., 90-day mortality). Also as described herein, particular cargos can be present within EVs (e.g., circulating EVs) in a sample obtained from a mammal (e.g., a human) having an ALD. For example, EVs from a sample (e.g., a plasma sample) obtained from a mammal can be assessed to determine if the mammal has an ALD based, at least in part, on the presence, absence, or level of one or more cargos present within the EVs.

This document also provides methods and materials for treating a mammal having an ALD (e.g., AH). For example, a mammal identified as having an ALD as described herein (e.g., based, at least in part, on the presence of an elevated level of EVs and/or the presence, absence, or level of one or more cargos in the EVs in a sample from the mammal) can be administered one or more IL-22 polypeptides, one or more inhibitors of a BRD4 polypeptide, and/or one or more ALD treatments to treat the mammal.

In some cases, a mammal can be identified as having an ALD (e.g., AH) based, at least in part, on the presence of an elevated level of EVs (e.g., circulating EVs) in a sample (e.g., a sample obtained from a mammal such as a human). The term “elevated level” as used herein with respect to a level of EVs refers to any level that is greater than a reference level of EVs. The term “reference level” as used herein with respect to EVs refers to the level of EVs typically observed in a sample (e.g., a control sample) from one or more comparable mammals (e.g., humans of comparable age) that do not have an ALD. Control samples can include, without limitation, samples from healthy mammals, samples from mammals that are heavy alcohol drinkers, samples from mammals that have decompensated alcoholic cirrhosis, or samples from mammals that have a non-alcohol related liver disease (e.g., a Model for End Stage Liver Disease (MELD) score matched non-alcohol related liver disease) such as NASH, PBC, and PSC. In some cases, an elevated level of EVs can be a level that is greater than about >1.2×10¹¹ EVs/mL of sample (e.g., plasma). For example, an elevated level of EVs can be a level that is from about 1.2×10¹¹ EVs to about 2.5×10¹² EVs/mL of sample (e.g., plasma). It will be appreciated that levels from comparable samples are used when determining whether or not a particular level is an elevated level.

An EV can be any type of EV. Examples of EVs include, without limitation, exosomes, microvesicles, ectosomes, microparticles, oncosomes, and apoptotic bodies. An EV can be any appropriate size. In some cases, EVs can have a size (e.g., a longest diameter) of from about 20 nanometers (nm) to about 10000 nm. For example, EVs can be small EVs (sEVs) having a size (e.g., a longest diameter) of about 40 nm to about 100 nm. In some cases, EVs can be medium EVs (mEVs) having a size (e.g., a longest diameter) of about 100 nm to about 200 nm. In some cases, EVs can be large EVs (lEVs) having a size (e.g., a longest diameter) of about 200 nm to about 10000 nm.

Any appropriate method can be used to detect the presence, absence, or level of EVs within a sample (e.g., a sample obtained from a mammal such as a human). For example, nano-tracking analysis (NTA), nano-scale flow cytometry, nano-plasmon enhanced scattering (nPES) assay, nano-fluidic devices, differential ultracentrifugation, antigen-based isolation, and/or size exclusion methods can be used to determine the presence, absence, or level of EVs in a sample. In some cases, the presence, absence, or level of EVs within a sample can be detected without enriching the EVs within the sample. In some cases, the presence, absence, or level of EVs within a sample can be determined as described in Example 4. In some cases, the presence, absence, or level of EVs within a sample can be determined as described elsewhere (see, e.g., Hirsova et al., Gastroenterology; 150:956-967 (2016); Kakazu et al., Journal of Lipid Research; 57:233-245 (2016); and Verma et al., Journal of Hepatology; 64:651-660 (2016)).

In some cases, a mammal (e.g., a human such as a human suspected as having an ALD such as AH) can be identified as having an ALD based, at least in part, on the presence, absence, or level of one or more (e.g., one, two, three, four, five, six, seven, eight, nine, or more) cargos in the EVs (e.g., circulating EVs) in a sample (e.g., a sample obtained from the mammal). A cargo can by any appropriate type of cargo (e.g., lipids such as sphingolipids, microRNAs, non-coding RNAs, and polypeptides such as enzymes). Examples of cargos that can be present within EVs in a sample from a mammal having an ALD include, without limitation, SPH, SPA, C14-cer, C16-cer, C24-cer, S1P, C18:1 ceramide, C18:0 ceramide, C20:0 ceramide, C22:0 ceramide, C24:0 ceramide, and C24:1 ceramide. In some cases, a cargo within an EV can be from a cell from which the EV was derived (e.g., the parent cell).

In some cases, a mammal (e.g., a human such as a human suspected as having an ALD such as AH) can be identified as having an ALD based, at least in part, on the presence of one or more enriched cargos in EVs (e.g., circulating EVs) in a sample (e.g., a sample obtained from a mammal such as a human). The term “enriched” as used herein with respect to a level of a cargo present within an EV refers to any level that is greater than a reference level of that cargo within EVs. The term “reference level” as used herein with respect to a level of a cargo present within an EV refers to the level of that cargo present within an EV typically observed in EVs in a sample (e.g., a control sample) from one or more comparable mammals (e.g., humans of comparable age) that do not have an ALD. Control samples can include, without limitation, samples from healthy mammals, samples from mammals that are heavy drinkers, samples from mammals that have decompensated alcoholic cirrhosis, and samples from mammals that have a non-alcohol related liver disease (e.g., MELD score matched non-alcohol related liver disease such as NASH, PBC, and PSC). In some cases, an enriched cargo present within an EV can be present at a level that is at least 1.7 (e.g., at least 2, at least 2.5, at least 2.7, at least 3, at least 3.5, at least 4, at least 4.6, at least 5, at least 6, at least 7, at least 7.5, at least 8, at least 9, at least 9.8, at least 10, at least 11.4, at least 12.5, at least 13.2, at least 14, at least 15, or at least 16.6) fold greater than a reference level of that cargo. It will be appreciated that levels from comparable samples are used when determining whether or not a particular cargo is enriched.

In some cases, an enriched level of SPH within EVs in an AH sample can be a level that is from about 2.1 nM to about 55.96 nM (e.g., from about 2.1 nM to about 50 nM, from about 2.1 nM to about 40 nM, from about 2.1 nM to about 30 nM, from about 2.1 nM to about 20 nM, from about 5 nM to about 55.96 nM, from about 10 nM to about 55.96 nM, from about 15 nM to about 55.96 nM, from about 5 nM to about 50 nM, from about 7 nM to about 40 nM, from about 10 nM to about 30 nM, from about 15 nM to about 20 nM, from about 10 nM to about 20 nM, from about 12 nM to about 23 nM, from about 15 nM to about 25 nM, or from about 17 nM to about 30 nM). For example, an enriched level of SPH within EVs in a sample can be a level that is about 18 nM (e.g., 18.21 nM) of sample (e.g., plasma).

In some cases, an enriched level of SPA within EVs in a sample can be a level that is from about 0.9 nM to about 34.02 nM (e.g., from about 0.9 nM to about 30 nM, from about 0.9 nM to about 25 nM, from about 0.9 nM to about 20 nM, from about 0.9 nM to about 15 nM, from about 0.9 nM to about 10 nM, from about 1 nM to about 34.02 nM, from about 2 nM to about 34.02 nM, from about 3 nM to about 34.02 nM, from about 4 nM to about 34.02 nM, from about 5 nM to about 34.02 nM, from about 1 nM to about 25 nM, from about 2 nM to about 20 nM, from about 3 nM to about 15 nM, from about 4 nM to about 10 nM, from about 1 nM to about 7 nM, from about 2 nM to about 8 nM, from about 3 nM to about 9 nM, from about 4 nM to about 10 nM, or from about 5 nM to about 12 nM). For example, an enriched level of SPA within EVs in a sample can be a level that is about 7 nM (e.g., 6.75 nM) of sample (e.g., plasma).

In some cases, an enriched level of S1P within EVs in an AH sample can be a level that is from about 14.98 nM to about 226.57 nM (e.g., from about 14.98 nM to about 200 nM, from about 14.98 nM to about 150 nM, from about 14.98 nM to about 125 nM, from about 14.98 nM to about 100 nM, from about 14.98 nM to about 75 nM, from about 20 nM to about 226.57 nM, from about 25 nM to about 226.57 nM, from about 35 nM to about 226.57 nM, from about 50 nM to about 226.57 nM, from about 15 nM to about 200 nM, from about 20 nM to about 150 nM, from about 25 nM to about 125 nM, from about 50 nM to about 100 nM, from about 25 nM to about 75 nM, from about 35 nM to about 85 nM, from about 45 nM to about 95 nM, or from about 55 nM to about 105 nM). For example, an enriched level of S1P within EVs in a sample can be a level that is about 70 nM (e.g., 68.10 nM) of sample (e.g., plasma).

In some cases, an enriched level of C14-cer within EVs in a sample can be a level that is from about 0.25 nM to about 54.7 nM (e.g., from about 0.25 nM to about 50 nM, from about 0.25 nM to about 40 nM, from about 0.25 nM to about 30 nM, from about 0.25 nM to about 20 nM, from about 0.25 nM to about 10 nM, from about 0.25 nM to about 5 nM, from about 1 nM to about 54.7 nM, from about 2 nM to about 54.7 nM, from about 3 nM to about 54.7 nM, from about 4 nM to about 54.7 nM, from about 0.5 nM to about 50 nM, from about 1 nM to about 35 nM, from about 2 nM to about 25 nM, from about 3 nM to about 15 nM, from about 4 nM to about 10 nM, from about 2 nM to about 5 nM, from about 3 nM to about 6 nM, or from about 4 nM to about 7 nM). For example, an enriched level of C14-cer within EVs in a sample can be a level that is about 5 nM (e.g., 4.56 nM) of sample (e.g., plasma).

In some cases, an enriched level of C16-cer within EVs in a sample can be a level that is from about 19 nM to about 1765.9 nM (e.g., from about 25 nM to about 1765.9 nM, from about 50 nM to about 1765.9 nM, from about 100 nM to about 1765.9 nM, from about 150 nM to about 1765.9 nM, from about 200 nM to about 1765.9 nM, from about 250 nM to about 1765.9 nM, from about 19 nM to about 1500 nM, from about 19 nM to about 1250 nM, from about 19 nM to about 1000 nM, from about 19 nM to about 750 nM, from about 19 nM to about 500 nM, from about 19 nM to about 400 nM, from about 19 nM to about 300 nM, from about 19 nM to about 250 nM, from about 25 nM to about 1500 nM, from about 50 nM to about 1000 nM, from about 75 nM to about 500 nM, from about 100 nM to about 300 nM, from about 20 nM to about 100 nM, from about 100 nM to about 200 nM, or from about 200 nM to about 300 nM). For example, an enriched level of C16-cer within EVs in a sample can be a level that is about 225 nM (e.g., 226.48 nM) of sample (e.g., plasma).

In some cases, an enriched level of C18:1-cer within EVs in an AH sample can be a level that is from about 0.07 nM to about 18 nM (e.g., from about 0.1 nM to about 18 nM, from about 0.5 nM to about 18 nM, from about 0.75 nM to about 18 nM, from about 1 nM to about 18 nM, from about 1.2 nM to about 18 nM, from about 1.3 nM to about 18 nM, from about 1.4 nM to about 18 nM, from about 1.5 nM to about 18 nM, from about 0.07 nM to about 15 nM, from about 0.07 nM to about 10 nM, from about 0.07 nM to about 7 nM, from about 0.07 nM to about 5 nM, from about 0.07 nM to about 4 nM, from about 0.07 nM to about 3 nM, from about 0.07 nM to about 2 nM, from about 0.8 nM to about 15 nM, from about 0.9 nM to about 12 nM, from about 1 nM to about 10 nM, from about 1.1 nM to about 7 nM, from about 1.2 nM to about 5 nM, from about 1.3 nM to about 2 nM, from about 0.1 nM to about 2 nM, or from about 0.5 nM to about 2.5 nM). For example, an enriched level of C18:1-cer within EVs in a sample can be a level that is about 1 nM (e.g., 1.50 nM) of sample (e.g., plasma).

In some cases, an enriched level of C18-cer within EVs in an AH sample can be a level that is from about 2.07 nM to about 991.1 nM (e.g., from about 5 nM to about 991.1 nM, from about 10 nM to about 991.1 nM, from about 25 nM to about 991.1 nM, from about 50 nM to about 991.1 nM, from about 2.07 nM to about 750 nM, from about 2.07 nM to about 500 nM, from about 2.07 nM to about 250 nM, from about 2.07 nM to about 100 nM, from about 2.07 nM to about 75 nM, from about 10 nM to about 750 nM, from about 15 nM to about 500 nM, from about 20 nM to about 250 nM, from about 25 nM to about 100 nM, from about 35 nM to about 90 nM, from about 50 nM to about 75 nM, from about 25 nM to about 75 nM, from about 30 nM to about 70 nM, or from about 40 nM to about 80 nM). For example, an enriched level of C18-cer within EVs in a sample can be a level that is about 70 nM (e.g., 67.56 nM) of sample (e.g., plasma).

In some cases, an enriched level of C20-cer within EVs in an AH sample can be a level that is from about 1.49 nM to about 423.3 nM (e.g., from about 1.49 nM to about 350 nM, from about 1.49 nM to about 250 nM, from about 1.49 nM to about 150 nM, from about 1.49 nM to about 100 nM, from about 1.49 nM to about 75 nM, from about 1.49 nM to about 50 nM, from about 3 nM to about 423.3 nM, from about 5 nM to about 423.3 nM, from about 10 nM to about 423.3 nM, from about 15 nM to about 423.3 nM, from about 20 nM to about 423.3 nM, from about 25 nM to about 423.3 nM, from about 2.5 nM to about 250 nM, from about 3 nM to about 200 nM, from about 5 nM to about 150 nM, from about 10 nM to about 100 nM, from about 15 nM to about 75 nM, from about 25 nM to about 50 nM, from about 15 nM to about 40 nM, from about 20 nM to about 50 nM, from about 25 nM to about 55 nM, or from about 30 nM to about 60 nM). For example, an enriched level of C20-cer within EVs in a sample can be a level that is about 35 nM (e.g., 34.54 nM) of sample (e.g., plasma).

In some cases, an enriched level of C22-cer within EVs in an AH sample can be a level that is from about 10.3 nM to about 1359.64 nM (e.g., from about 10.3 nM to about 1000 nM, from about 10.3 nM to about 750 nM, from about 10.3 nM to about 500 nM, from about 10.3 nM to about 250 nM, from about 10.3 nM to about 200 nM, from about 25 nM to about 1359.64 nM, from about 50 nM to about 1359.64 nM, from about 75 nM to about 1359.64 nM, from about 100 nM to about 1359.64 nM, from about 125 nM to about 1359.64 nM, from about 15 nM to about 1000 nM, from about 25 nM to about 750 nM, from about 50 nM to about 500 nM, from about 75 nM to about 250 nM, from about 100 nM to about 200 nM, from about 25 nM to about 175 nM, from about 50 nM to about 200 nM, from about 75 nM to about 225 nM, from about 100 nM to about 250 nM, or from about 125 nM to about 300 nM). For example, an enriched level of C22-cer within EVs in a sample can be a level that is about 150 nM (e.g., 154.38 nM) of sample (e.g., plasma).

In some cases, an enriched level of C24:1-cer within EVs in an AH sample can be a level that is from about 20.3 nM to about 2856.03 nM (e.g., from about 20.3 nM to about 2500 nM, from about 20.3 nM to about 2000 nM, from about 20.3 nM to about 1500 nM, from about 20.3 nM to about 1000 nM, from about 20.3 nM to about 750 nM, from about 20.3 nM to about 500 nM, from about 20.3 nM to about 400 nM, from about 50 nM to about 2856.03 nM, from about 150 nM to about 2856.03 nM, from about 200 nM to about 2856.03 nM, from about 250 nM to about 2856.03 nM, from about 300 nM to about 2856.03 nM, from about 25 nM to about 2000 nM, from about 50 nM to about 1500 nM, from about 100 nM to about 1000 nM, from about 150 nM to about 750 nM, from about 200 nM to about 500 nM, from about 50 nM to about 350 nM, from about 75 nM to about 375 nM, from about 100 nM to about 400 nM, from about 125 nM to about 425 nM, from about 150 nM to about 450 nM, or from about 300 nM to about 400 nM). For example, an enriched level of C24:1-cer within EVs in a sample can be a level that is about 325 nM (e.g., 326.75 nM) of sample (e.g., plasma).

In some cases, an enriched level of C24-cer within EVs in a sample can be a level that is from about 23.2 nM to about 2030.62 nM (e.g., from about 23.2 nM to about 1500 nM, from about 23.2 nM to about 1250 nM, from about 23.2 nM to about 1000 nM, from about 23.2 nM to about 750 nM, from about 23.2 nM to about 500 nM, from about 23.2 nM to about 300 nM, from about 50 nM to about 2030.62 nM, from about 100 nM to about 2030.62 nM, from about 150 nM to about 2030.62 nM, from about 200 nM to about 2030.62 nM, from about 250 nM to about 2030.62 nM, from about 25 nM to about 1500 nM, from about 50 nM to about 1000 nM, from about 100 nM to about 750 nM, from about 150 nM to about 500 nM, from about 200 nM to about 300 nM, from about 50 nM to about 275 nM, from about 75 nM to about 300 nM, from about 100 nM to about 325 nM, from about 150 nM to about 350 nM, or from about 200 nM to about 400 nM). For example, an enriched level of C24-cer within EVs in a sample can be a level that is about 270 nM (e.g., 265.81 nM) of sample (e.g., plasma).

In some cases, a mammal can be identified as having an ALD (e.g., AH) based, at least in part, on enriched SPH, enriched SPA, enriched S1P, enriched C14-cer, enriched C16-cer, enriched C18-cer, enriched C20-cer, enriched C22-cer, enriched C24:1-cer, and enriched C24-cer in EVs (e.g., circulating EVs) in a sample from the mammal.

In some cases, a mammal can be identified as having an ALD (e.g., AH) based, at least in part, on enriched SPA, enriched C16-cer, enriched C18:1-cer, enriched C18-cer, enriched C20-cer, enriched C22-cer, enriched C24:1-cer, and enriched C24-cer in EVs (e.g., circulating EVs) in a sample from the mammal.

In some cases, a mammal can be identified as having an ALD (e.g., AH) based, at least in part, on enriched SPH, enriched SPA, enriched S1P, enriched C16-cer, enriched C20-cer, enriched C22-cer, enriched C24:1-cer, and enriched C24-cer in EVs (e.g., circulating EVs) in a sample from the mammal.

In some cases, a mammal can be identified as having an ALD (e.g., AH) based, at least in part, on enriched SPH, enriched SPA, enriched S1P, enriched C16-cer, enriched C20-cer, enriched C22-cer, enriched C24:1-cer, and enriched C24-cer in EVs (e.g., circulating EVs) in a sample from the mammal.

Any appropriate method can be used to determine the presence, absence, or level of one or more cargos in EVs (e.g., circulating EVs) within a sample (e.g., a sample obtained from a mammal such as a human). For example, transmission electron microscopy, mass spectrometry techniques (e.g., tandem mass spectroscopy techniques such as LC/MS), and/or biochemical assays can be used to determine if the EVs contain one or more lipids. For example, immunoassays (e.g., immunohistochemistry (IHC) techniques and western blotting techniques), mass spectrometry techniques (e.g., proteomics-based mass spectrometry assays), transmission electron microscopy, and/or enzyme-linked immunosorbent assays (ELISAs) can be used to determine if the EVs contain one or more polypeptides. In some cases, the presence, absence, or level of one or more cargos in EVs within a sample can be determined as described in Example 1, Example 2, Example 3, and/or Example 4. In some cases, the presence, absence, or level of one or more cargos in EVs within a sample can be determined as described elsewhere (see, e.g., Li et al., Hepatology Communications; 3(9):1235-1249 (2019).

Any appropriate mammal (e.g., a mammal suspected as having an ALD such as AH) can be assessed and/or treated as described herein. Examples of mammals that can be assessed and/or treated as described herein include, without limitation, humans, non-human primates such as monkeys, dogs, cats, horses, cows, pigs, sheep, mice, and rats. In some cases, a human can be assessed and/or treated as described herein.

Any appropriate sample from a mammal (e.g., a human such as a human suspected as having an ALD such as AH) can be assessed as described herein (e.g., for the presence, absence, or level of EVs and/or for the presence, absence, or level of one or more cargos in EVs in the sample). In some cases, a sample can be a biological sample. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, fluid samples (e.g., whole blood, serum, plasma, PBMCs, urine, and CSF), tissue samples, saliva, tears, and lymph. A sample can be a fresh sample or a fixed sample (e.g., EDTA plasma, citrate plasma, and heparinized plasma). In some cases, a sample can be a processed sample (e.g., an embedded sample such as a paraffin or OCT embedded sample, or processed to isolate or extract one or more biological molecules). For example, a blood (e.g., plasma) sample can be obtained from a mammal and can be assessed for the presence, absence, or level of EVs to determine if the mammal has an ALD based, at least in part, on the presence of an elevated level of EVs in the sample. In some cases, a blood (e.g., plasma) sample can be obtained from a mammal and can be assessed for presence, absence, or level of EVs and/or for the presence, absence, or level of one or more cargos in EVs within the sample to determine if the mammal has an ALD.

In some cases, after a mammal (e.g., a human such as a human suspected as having an ALD such as AH) is identified as having an ALD (e.g., AH) based, at least in part, on an elevated level of EVs (e.g., circulating EVs) and/or the presence of one or more enriched cargos (e.g., enriched SPA, enriched C14-cer, enriched C16-cer, and enriched C24-cer) in the EVs in a sample as described herein, the presence of an ALD can be confirmed using one or more diagnostic techniques. Any appropriate technique can be used to confirm that a mammal has an ALD. For example, liver function tests (e.g., blood tests for levels of certain enzymes and proteins such as ALT, AST, alkaline phosphatase (ALP), albumin, bilirubin, gamma-glutamyltransferase (GGT), L-lactate dehydrogenase (LD), and prothrombin time (PT)), imaging of the liver (e.g., imaging of the liver using ultrasound, computerized tomography (CT) scanning, and/or magnetic resonance imaging (MRI)), and liver tissue analysis (e.g., liver tissue analysis of a liver biopsy) can be used to confirm that the mammal has an ALD.

In some cases, a mammal (e.g., a human such as a human suspected as having an ALD such as AH) identified as having an ALD based, at least in part, on the presence of an elevated level of EVs (e.g., circulating EVs) and/or the presence, absence, or level of one or more cargos in EVs in a sample obtained from a mammal can be assessed for an increased risk of mortality (e.g., an increased risk of mortality due to the ALD). For example, a mammal can be assessed for an elevated level of EVs in a sample that is from about 1.2×10¹¹ to about 5×10¹¹ EVs/mL of sample (e.g., plasma). A mammal having an elevated level of EVs in a sample that is from about 1.2×10¹¹ to about 5×10¹¹ EVs/mL can be identified as having a low risk of mortality (e.g., a low risk of mortality within about 90 days). For example, a mammal can be assessed for an elevated level of EVs in a sample that is greater than about 5×10¹¹ EVs/mL of sample (e.g., plasma). A mammal having an elevated level of EVs in a sample that is greater than about 5×10¹¹ EVs/mL of sample can be identified as having a high risk of mortality (e.g., a high risk of mortality within about 90 days). In some cases, a mammal having an elevated level of EVs in a sample that is from about 5×10¹¹ to about 2.5×10¹² EVs/mL of sample (e.g., plasma) can be identified as having a high risk of mortality (e.g., a high risk of mortality within about 90 days).

In some cases, after a mammal (e.g., a human such as a human suspected as having an ALD such as AH) is identified as having an ALD (e.g., AH) and as having a high risk of mortality (e.g., a high risk of mortality within about 90 days) based, at least in part, on an elevated level of EVs (e.g., circulating EVs) as described herein, the prognosis (e.g., the risk of mortality) of the ALD, the disease severity, and/or the treatment futility can be confirmed using one or more scoring methods. Any appropriate prognostic method can be used to assess the risk of mortality in a mammal having an ALD. For example, static and dynamic scores such as a MELD score, Maddrey's Discriminant Function (MDF), and Lille score can be used to assess the risk of mortality, severity, and/or treatment futility in a mammal having an ALD. In some cases, the risk of mortality in a mammal having an ALD can be assessed as described elsewhere (see, e.g., Dunn et al., Hepatology; 41:353-358 (2005); Maddrey et al., Gastroenterology; 75:193-199 (1978); Kamath et al., Hepatology; 45:797-805 (2007); and Louvet et al., Hepatology; 45:1348-1354 (2007)).

When assessing and/or treating an ALD as described herein, the ALD can be any type of ALD. Examples of ALDs that can assessed and/or treated as described herein include, without limitation, AH, fatty liver, chronic hepatitis (e.g., chronic hepatitis with liver fibrosis), reversible steatosis, steatohepatitis, and cirrhosis. In some cases, an ALD can be as described elsewhere (see, e.g., Gao et al., Gastroenterology; 141:1572-1585 (2011)).

In some cases, after identifying a mammal (e.g., a human such as a human suspected as having an ALD such as AH) as having an ALD (e.g., AH) based, at least in part, on an elevated level of EVs (e.g., circulating EVs) and/or the presence of one or more enriched cargos (e.g., enriched SPA, enriched C14-cer, enriched C16-cer, and enriched C24-cer) in the EVs in a sample as described herein, the mammal can be administered or instructed to self-administer one or more IL-22 polypeptides (e.g., a composition including one or more IL-22 polypeptides) and/or one or more nucleic acids encoding an IL-22 polypeptide (e.g., a composition including one or more nucleic acids encoding an IL-22 polypeptide). Any appropriate IL-22 polypeptide(s) (and/or any appropriate nucleic acids encoding an IL-22 polypeptide) can be administered to a mammal as described herein. Examples of IL-22 polypeptides and nucleic acid sequences encoding IL-22 polypeptides can include, without limitation, those set forth in National Center for Biotechnology Information (NCBI) accession no. Q9GZX6, version Q9GZX6.1; accession no. NP_065386, version NP_065386.1; accession no. AAH69308, version AAH69308.1; and accession AAH70261, version AAH70261.1.

In some cases, after identifying a mammal (e.g., a human such as a human suspected as having an ALD such as AH) as having an ALD (e.g., AH) based, at least in part, on an elevated level of EVs (e.g., circulating EVs) and/or the presence of one or more enriched cargos (e.g., enriched SPA, enriched C14-cer, enriched C16-cer, and enriched C24-cer) in the EVs in a sample as described herein, the mammal can be administered or instructed to self-administer one or more inhibitors of a BRD4 polypeptide. An inhibitor of a BRD4 polypeptide can be an inhibitor of BRD4 polypeptide activity or an inhibitor of BRD4 polypeptide expression. In some cases, an inhibitor of a BRD4 polypeptide can be an inhibitor of the bromodomain 1 (BD1) of the BRD4 polypeptide. In some cases, an inhibitor of a BRD4 polypeptide can be an inhibitor of the BD2 of the BRD4 polypeptide. In some cases, an inhibitor of a BRD4 polypeptide can be an inhibitor of both the BD1 and the BD2 of the BRD4 polypeptide. Examples of inhibitors of a BRD4 polypeptide that can be used as described herein include, without limitation, iBET151, RVX-208, iBET 762, Zen-3694, JQ1, OTX-015, GSK620, and ABBV-744.

In some cases, once identified as having as having an ALD (e.g., AH) based, at least in part, on an elevated level of EVs (e.g., circulating EVs) and/or the presence of one or more enriched cargos (e.g., enriched SPA, enriched C14-cer, enriched C16-cer, and enriched C24-cer) in the EVs in a sample as described herein, a mammal can be administered or instructed to self-administer one or more ALD treatments. Treatments for ALD include, without limitation, administering nutritional supplements, attending alcohol cessation counseling (e.g., to reduce or eliminate alcohol consumption), administering corticosteroids (e.g., glucocorticoids such as prednisolone), administering pentoxifylline, and undergoing liver transplantation.

When a mammal having an ALD (e.g., AH) is identified as being at low risk of mortality based, at least in part, on the level of EVs (e.g., circulating EVs) in a sample as described herein, the mammal can be administered one or more IL-22 polypeptides, can be administered one or more inhibitors of a BRD4 polypeptide, can be administered nutritional supplements, and/or can attend alcohol cessation counseling (e.g., to reduce or eliminate alcohol consumption).

When a mammal having an ALD (e.g., AH) is identified as being at high risk of mortality (e.g., a high risk of mortality within about 90 days) based, at least in part, on an elevated level of EVs (e.g., circulating EVs) in a sample as described herein, the mammal can be administered one or more IL-22 polypeptides, can be administered one or more inhibitors of a BRD4 polypeptide, can be administered tEVs, can be administered corticosteroids (e.g., glucocorticoids such as prednisolone), can be administered pentoxifylline, and/or can undergo a liver transplant. A mammal having an ALD and identified as being at high risk of mortality also can be monitored for response to the one or more ALD treatments. For example, the level of EVs in a sample (e.g., a plasma sample) obtained from a mammal can be assessed about every 4 weeks (e.g., about 4 weeks after being administered one or more IL-22 polypeptides and/or one or more ALD treatments). In cases where the level of EVs in a sample obtained from a mammal has been reduced (e.g., reduced by about 3-fold as compared to a previously determined level) about 4 weeks after administering one or more IL-22 polypeptides and/or one or more ALD treatments, the mammal can be administered or instructed to self-administer a subsequence round of one or more IL-22 polypeptides and/or one or more ALD treatments. This cycle of assessing and treating can be repeated any appropriate number of times (e.g., two, three, four, or more times). In cases where the level of EVs in a sample obtained from a mammal has not been reduced (e.g., has not been reduced by about 3-fold as compared to a previously determined level, has remained the same as compared to a previously determined level, or has increased as compared to a previously determined level) about 4 weeks after administering one or more IL-22 polypeptides and/or one or more ALD treatments, the mammal can be classified as being in need of a liver transplant.

When treating a mammal (e.g., a human) having an ALD (e.g., AH) as described herein, the treatment can be effective to reduce the severity of one or more symptoms of the ALD. Symptoms of an ALD can include, without limitation, anorexia, weight loss, abdominal pain, abdominal distention, nausea, vomiting, hepatomegaly, jaundice, angiomas (e.g., spider angiomas), fever, encephalopathy, thrombocytopenia, hypoalbuminemia, coagulopathy, fatigue, weakness, and liver failure. For example, a treatment described herein can be effective to reduce the severity of one or more symptoms of ALD in a mammal having an ALD by, for example, 10, 20, 30, 40, 50, 60, 70, 80, 90, 95, or more percent.

When treating a mammal (e.g., a human) having an ALD (e.g., AH) as described herein, the treatment can be effective to reduce the severity of one or more complications associated with the ALD. Complications associated with an ALD can include, without limitation, enlarged veins (varices), ascites, hepatic encephalopathy, kidney failure, and infection. For example, a treatment described herein can be effective to reduce the severity of one or more complications associated with an ALD in a mammal having an ALD by, for example, 10, 20, 30, 40, 50, 60, 70, 80, 90, 95, or more percent.

When treating a mammal (e.g., a human) having an ALD (e.g., AH) as described herein, the treatment can be effective to increase the survival of the mammal. For example, a treatment described herein can be effective to increase the survival of a mammal having an ALD (e.g., having an ALD and identified as being at high risk of mortality) by, for example, 10, 20, 30, 40, 50, 60, 70, 80, 90, 95, or more percent.

The invention will be further described in the following examples, which do not limit the scope of the invention described in the claims.

### EXAMPLES

### Example 1: Circulating Extracellular Vesicles Carrying Sphingolipid Cargo for the Diagnosis and Dynamic Risk Profiling of AH

This Example investigated whether plasma EV concentration and sphingolipid cargo could serve as diagnostic biomarkers for AH and inform prognosis to permit dynamic risk profiling of AH subjects.

### Methods

### Sample Collection and Study Design

Blood samples from 36 heavy drinkers and 36 AH subjects were prospectively collected as reported elsewhere (see, e.g., Peeraphatdit T B, Kamath P S, Karpyak V M, Davis B, Desai V, Liangpunsakul S, Sanyal A, et al., Clin Gastroenterol Hepatol; 18(2):477-485 (2020)). Samples from 29 additional AH subjects were obtained. Twenty-nine additional subjects with severe end-stage liver disease (MELD score>20) due to etiologies other than alcohol-related liver disease, including non-alcoholic steatohepatitis (NASH), primary sclerosing cholangitis (PSC), and primary biliary cholangitis (PBC) were prospectively recruited and included as controls for MELD score>20 group. Samples from 30 decompensated alcoholic cirrhosis subjects were also obtained. Thirty-six healthy control samples were also included in the analysis. Eighteen AH subjects and 18 healthy controls were randomly included, and the remaining 25 AH subjects and 18 healthy controls included.

The diagnosis of heavy drinkers and AH was confirmed in the clinical setting by an experienced hepatologist using NIAAA criteria (see, e.g., Singal et al., Am J Gastroenterol; 113:175-194 (2018); and Crabb et al., Gastroenterology; 150:785-790 (2016)). Heavy alcohol drinking was defined as consumption of >40 grams of alcohol per day on average in women and >60 grams of alcohol per day on average in men for a minimum of 6 months and within 6 weeks prior to study enrollment. Criteria for inclusion in the heavy drinker group included: (1) AST, ALT, and total bilirubin levels within normal range; (2) no prior history of known ALD; and (3) absence of hepatosplenomegaly (from physical examination or radiographic imaging) or stigmata of liver disease. The diagnosis of AH was established following published criteria based on history of heavy alcohol consumption, clinical evaluation and appropriate laboratory testing (as defined as total bilirubin >2 mg/dL, ALT and AST >50 U/L but ≤500 U/L, and AST/ALT of ≥1.5). When a diagnosis of AH remained in question, a liver biopsy was pursued. Blood was collected in ethylenediaminetetraacetic acid tubes, and platelet poor plasma was separated. Plasma aliquots were stored at −80° C. EV isolation and analysis were performed in a blinded manner. Plasma aliquots were thawed only once, immediately before EV isolation.

Extraction of EVs from Plasma

Blinded plasma samples were used for isolation and analysis. Plasma samples were thawed on ice. 25 μL aliquots were set aside for sphingolipidomics from individual plasma samples. EVs were isolated from 800 μL of individual plasma samples. Samples were diluted 1:1 with sterile Dulbecco's phosphate-buffered saline (D-PBS) to reduce viscosity and centrifuged at 2000 g (Allegra 6R GH3.8) to remove cellular debris. The pellet was resuspended in sterile D-PBS and subjected to sequential ultracentrifugation at 12000 g and twice at 110,000 g (BC 344057 tubes; SW55Ti rotors; BC UTC Optima XPN-80, Beckman-Coulter, USA) to collect large and small EVs, respectively. Pellets were finally resuspended to 100 μL volume in sterile D-PBS (17, 30-33). 10 μL was aliquoted for nano-tracking analysis (NTA) and 90 μL for EV spingolipidomics. These were then immediately stored at −80° C. and thawed only once, at the time of analyses including NTA, sphingolipidomics, transmission electron microscopy (TEM), and western blot (WB).

### Assessment of EV Concentration in Plasma Samples by Nano-Tracking Analysis

NTA analysis was performed as described elsewhere (see, e.g., Hirsova et al., Gastroenterology; 150:956-967 (2016); Kakazu et al., Journal of Lipid Research; 57:233-245 (2016); and Verma et al., Journal of Hepatology; 64:651-660 (2016)). EV aliquots were diluted to a volume of 1000 μL with sterile D-PBS and analyzed for nanoparticle size distribution and concentration with a NanoSight NS300 (Malvern Instruments, Malvern, UK) equipped with a monochromatic 488-nm laser, an sCMOS camera, and a syringe pump. Several dilutions were assayed for each sample to fit within the optimal linear range of the instrument (10-40 particles/frame). Three consecutive 30-second videos with a rate of 20 frames per second were recorded for each sample and processed using the NTA 3.0 (build 0064) software (Malvern Instruments). EV counts were expressed per mL volume of plasma.

### Lipidomics Analyses of Plasma and Extracellular Vesicle Fractions

75 μL of the resuspended extracellular vesicles pellets were used for sphingolipidomic analyses as described elsewhere (see, e.g., Li et al., Hepatology Communications; 3(9):1235-1249 (2019). Ceramides were extracted from cell pellets suspended in PBS after the addition of internal standards and sonication. The extracts were measured against a standard curve on the Thermo TSQ Quantum Ultra mass spectrometer (West Palm Beach, Fla.) coupled with a Waters Acquity UPLC system (Milford, Mass.) as described in Li et al. (Hepatology Communications; 3(9):1235-1249 (2019). Briefly, cell pellets were suspended in 1×PBS, spiked with internal standard prior to extraction. The extracts were dried down and brought up in running prior to injecting on the LC/MS. Data was acquired under negative electrospray ionization condition.

### Statistical Analyses

Subjects were characterized into MELD score groups of ≤20 and >20 based on current consensus guidelines (see, e.g., Dunn et al., Hepatology; 41:353-358 (2005); Singal et al., Am J Gastroenterol; 113:175-194 (2018); and Crabb et al., Hepatology; doi:10.1002/hep.30866 (2020)). Continuous variables were summarized as mean or median and standard error as appropriate. Sample comparisons between groups using the Mann-Whitney U test, one-way ANOVA, or two-way ANOVA were performed as appropriate with post-test corrections when needed. The diagnostic accuracy parameters and associations were estimated using receiver operating characteristic (ROC) curve analysis. Area under the ROC curves (AUROC) with corresponding 95% confidence intervals (CI) and p values were calculated. Youden's index was used to establish a diagnostic cut-off in the discovery cohort where sensitivity and specificity reached maximum value with convergence. Kaplan-Meier (KM) survival curves were plotted to estimate the cumulative survival probability. The significance of observed differences and hazard ratios were calculated using logrank tests. The association between sphingolipids and clinical outcomes was determined using univariate Cox regression models. For statistical analyses and graphical and tabular preparations, GraphPad Prism 8 (GraphPad Software Inc, La Jolla, Calif.) and R-studio were used. R-packages pROC (for ROC curves); cutpointr (for Youden's index calculations); ggplot2 (for PCA plots); complexheatmap (for heatmap); and survival (for Kaplan-Meier curve analysis) were used.

### Results

### Characterization of Subjects and EVs

Subjects included healthy controls, heavy drinkers, end-stage liver disease controls with MELD score >20, decompensated alcoholic cirrhosis, and AH. Subjects with AH were divided into mild/moderate or severe based on recommended MELD score cut-off of 20. End stage liver disease controls had NASH cirrhosis or cirrhosis secondary to PSC or PBC. Subject characteristics are described in Table 1.

TABLE 1
Baseline characteristics of heavy drinkers, end-stage liver disease,
decompensated alcoholic cirrhosis, and alcoholic hepatitis subjects.
All values are mean ± SD.

| | | | Severe | Severe End- | |
|---|---|---|---|---|---|
| | | Alcoholic | Alcoholic | Stage Liver | Alcoholic |
| | | Hepatitis | Hepatitis | Disease | Cirrhosis |
| | Heavy | (MELD ≤ 20) | (MELD > 20) | (MELD > 20) | (MELD 6-38) |
| | Drinkers | (n = 23) | (n = 28) | (n = 29) | (n = 30) |
| Age (years) | 43.4 ± 9.9 | 51.2 ± 10.3 | 46.8 ± 8.9 | 54.4 ± 11.9 | 53.0 ± 7.3 |
| Albumin (g/dl) | 3.6 ± 0.4 | 3.0 ± 0.6 | 2.8 ± 0.5 | 3.1 ± 0.5 | 3.2 ± 0.7 |
| T. Bilirubin (mg/dl) | 0.6 ± 0.3 | 5.4 ± 4.3 | 23.2 ± 13.4 | 9.7 ± 7.7 | 14.4 ± 44.8 |
| Creatinine (mg/dl) | 0.8 ± 0.2 | 0.7 ± 0.2 | 1.9 ± 1.7 | 1.5 ± 0.8 | 1.1 ± 0.7 |
| ALT (IU/L) | 25.7 ± 9.3 | 92.3 ± 111.1 | 57.5 ± 51.7 | 78 ± 67.4 | 36.4 ± 30.8 |
| AST (IU/L) | 25.7 ± 9.3 | 149.7 ± 102.9 | 119.4 ± 65.2 | 120.2 ± 80.7 | 70.6 ± 84.6 |
| ALP (IU/L) | 67.5 ± 23.8 | 240.9 ± 142.9 | 227.9 ± 145.8 | 285.6 ± 267.1 | 289.9 ± 211.6 |
| T. Protein (g/dl) | 6.27 ± 0.5 | 6.0 ± 0.5 | 5.6 ± 0.7 | 7.4 ± 4.8 | 6.3 ± 0.7 |
| BMI* | 29.6 ± 7.8 | 26.6 ± 6.7 | 33.7 ± 7.7 | 31.2 ± 7.4 | — |
| CTP | 5.4 ± 0.5 | 8.6 ± 1.8 | 11.3 ± 1.2 | 10.2 ± 1.4 | 8.5 ± 1.8 |
| MELD | 6.4 ± 0.8 | 14.7 ± 3.6 | 31.0 ± 7.6 | 24.5 ± 4.7 | 16.0 ± 6.9 |

*BMI data not available for 22 alcoholic hepatitis subjects and 30 decompensated alcoholic cirrhosis subjects.

The concentration of EVs isolated from plasma ranged between 2.8×10⁹ and 2.5×10¹² EVs/mL, with an overall median value of 8.9×10¹⁰ particles/mL in all subjects (Table 2).

TABLE 2
Circulating EV Counts.

| EV | | | | | Decompensated | |
|---|---|---|---|---|---|---|
| concentration | Healthy | Heavy | | PBC and | Alcoholic | Alcoholic |
| (particles/mL) | Controls | Drinkers | NASH | PSC | Cirrhosis | Hepatitis |
| Minimum | 2.8 × 10⁹ | 1.25 × 10¹⁰ | 1.08 × 10¹⁰ | 1.8 × 10¹⁰ | 1.6 × 10¹⁰ | 7.65 × 10¹⁰ |
| 25^{th} Percentile | 1.91 × 10¹⁰ | 8.94 × 10¹⁰ | 2.6 × 10¹⁰ | 2.9 × 10¹⁰ | 5.18 × 10¹⁰ | 2.75 × 10¹¹ |
| Median | 4.38 × 10¹⁰ | 1.28 × 10¹¹ | 3.06 × 10¹⁰ | 6.29 × 10¹⁰ | 9.2 × 10¹⁰ | 5.38 × 10¹¹ |
| 75^{th} Percentile | 8.88 × 10¹⁰ | 1.91 × 10¹¹ | 5.74 × 10¹⁰ | 1.18 × 10¹¹ | 1.53 × 10¹¹ | 1.01 × 10¹² |
| Maximum | 3.63 × 10¹¹ | 4.68 × 10¹¹ | 1.76 × 10¹¹ | 1.98 × 10¹¹ | 1.1 × 10¹² | 2.5 × 10¹² |

Statistical comparisons of EV counts: AH vs. healthy controls (p <0.0001), AH vs. heavy drinkers (p = 0.0001), AH vs. NASH and cholestatic liver diseases (p <0.0001), AH vs. decompensated alcoholic cirrhosis (p <0.0001).

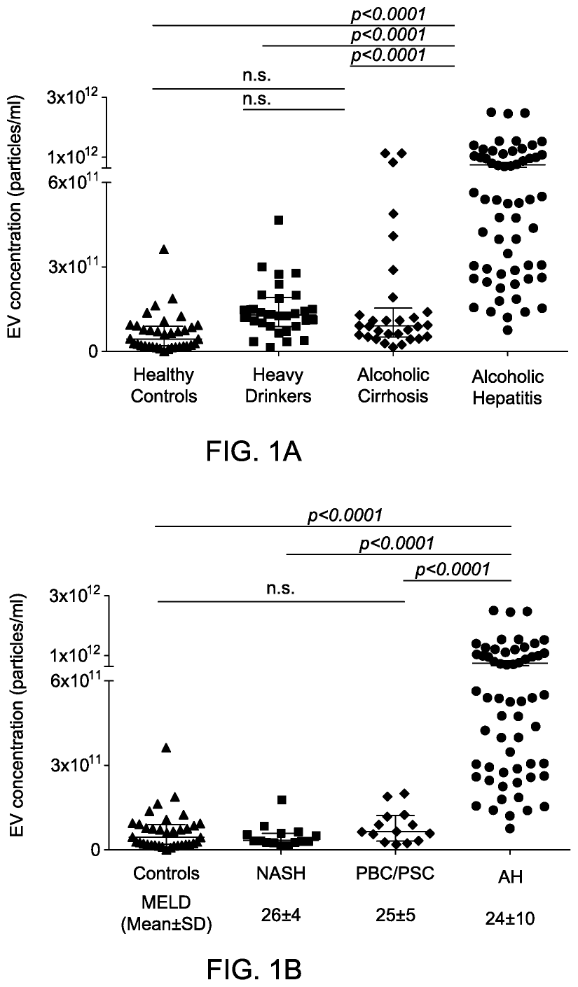

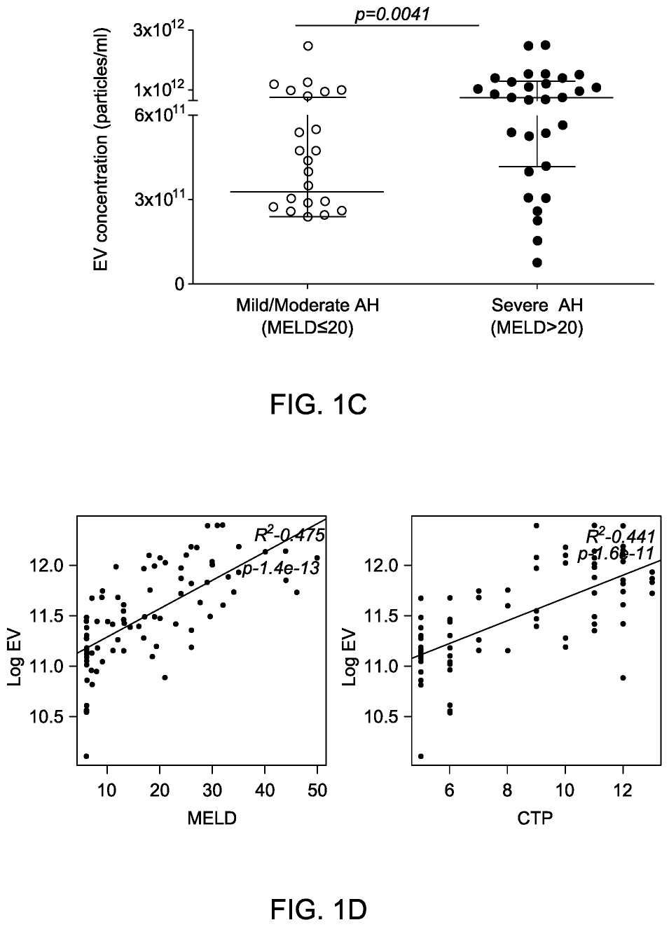

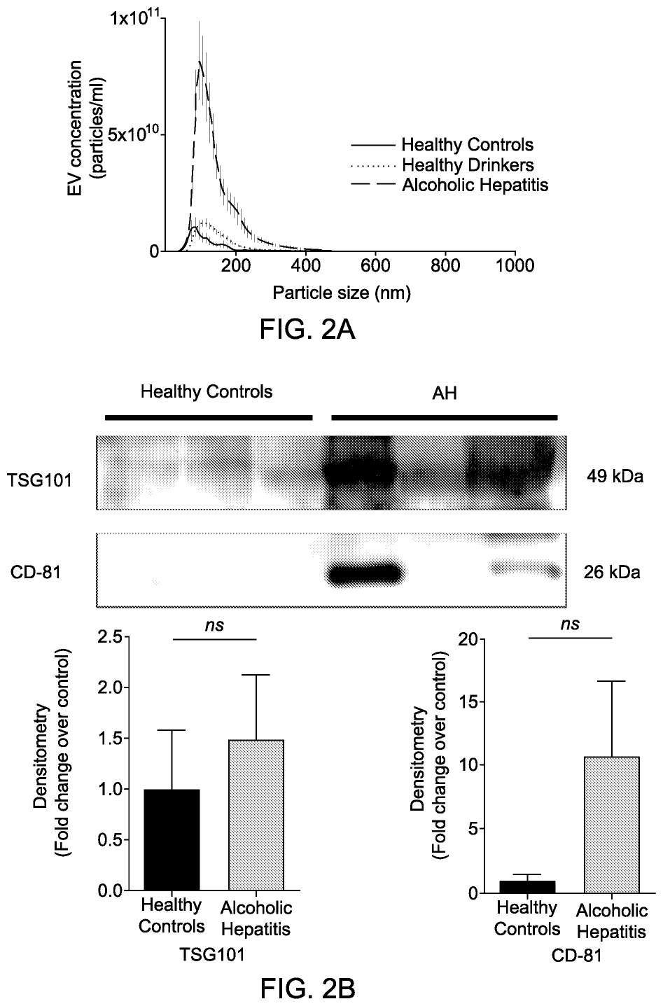

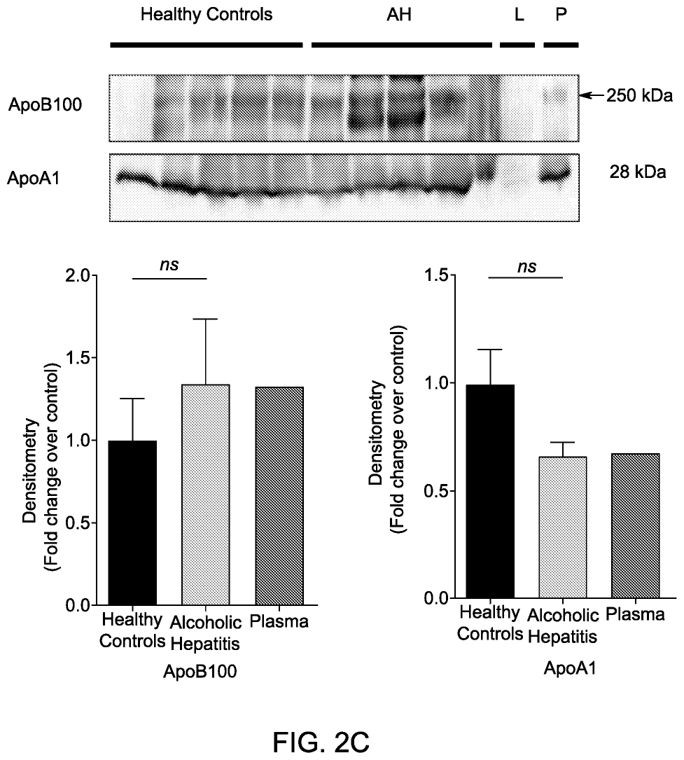

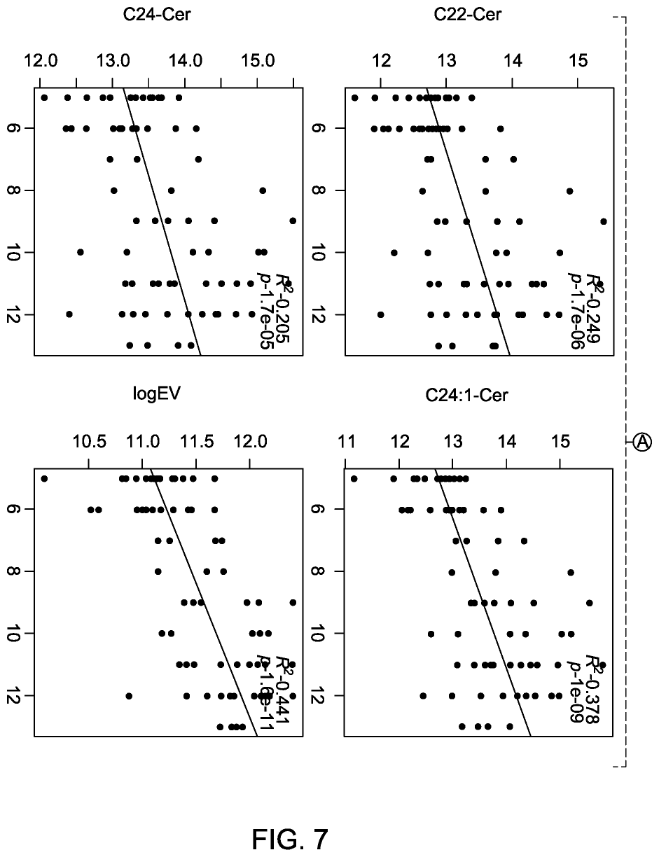

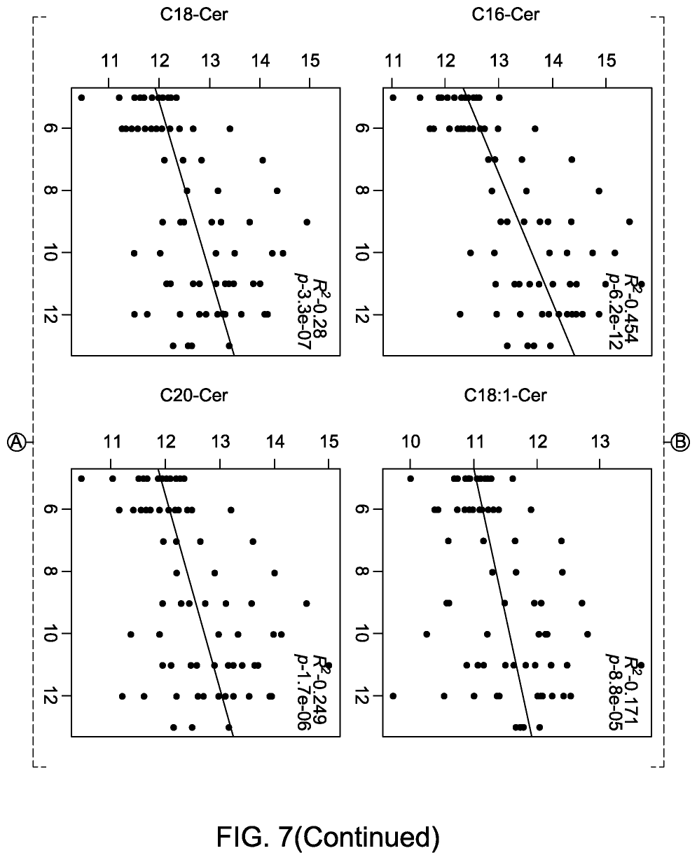

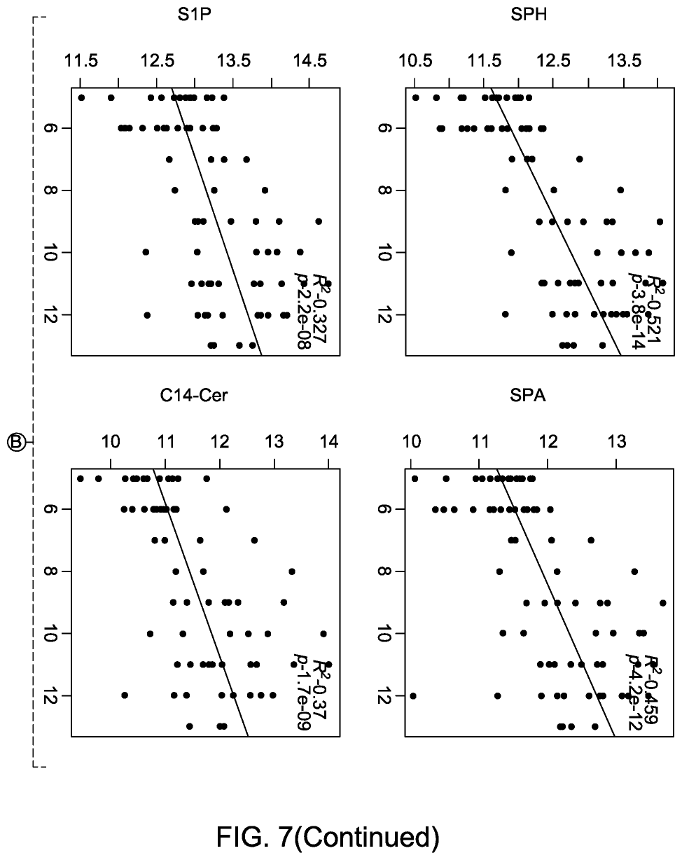

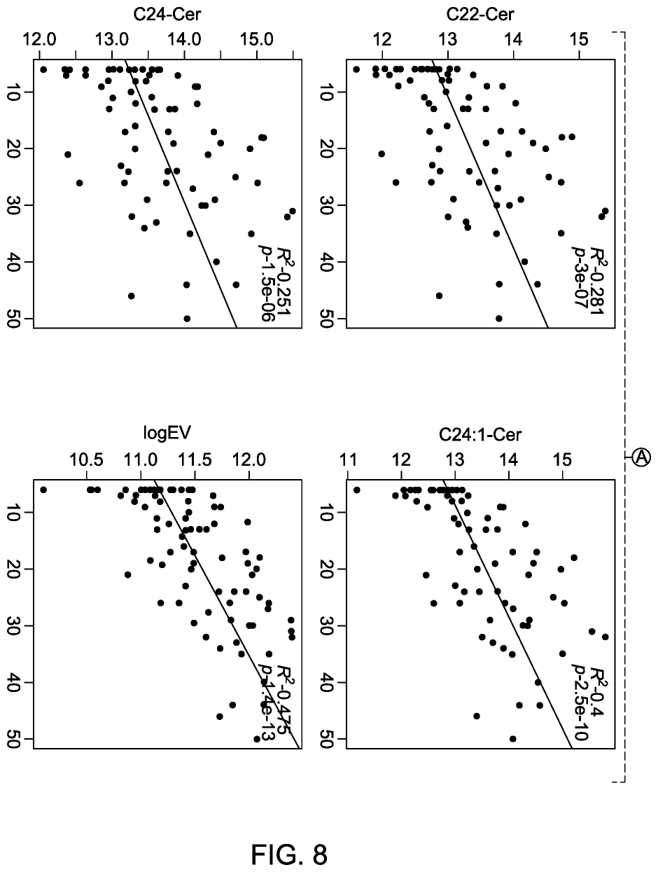

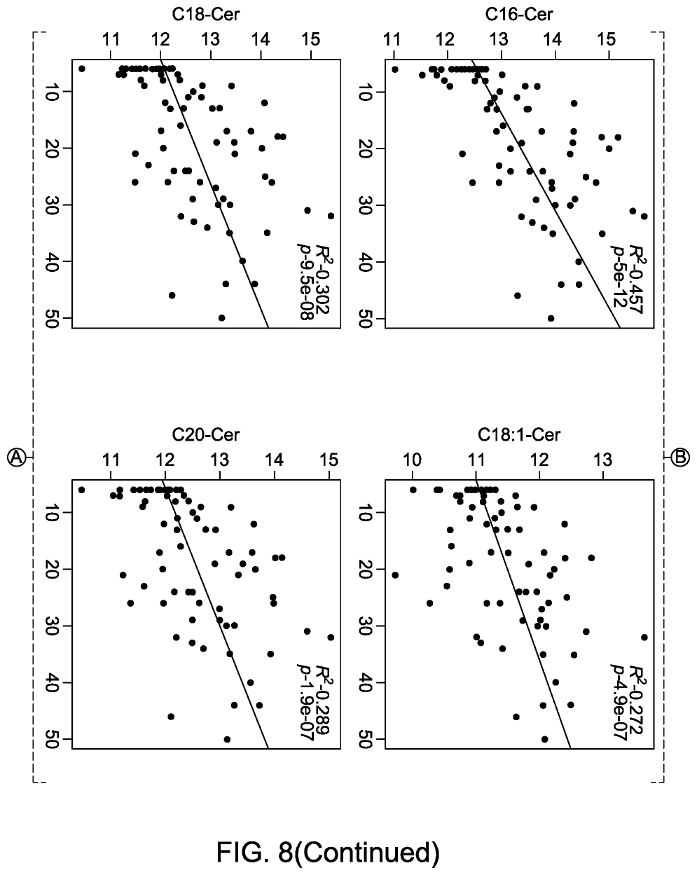

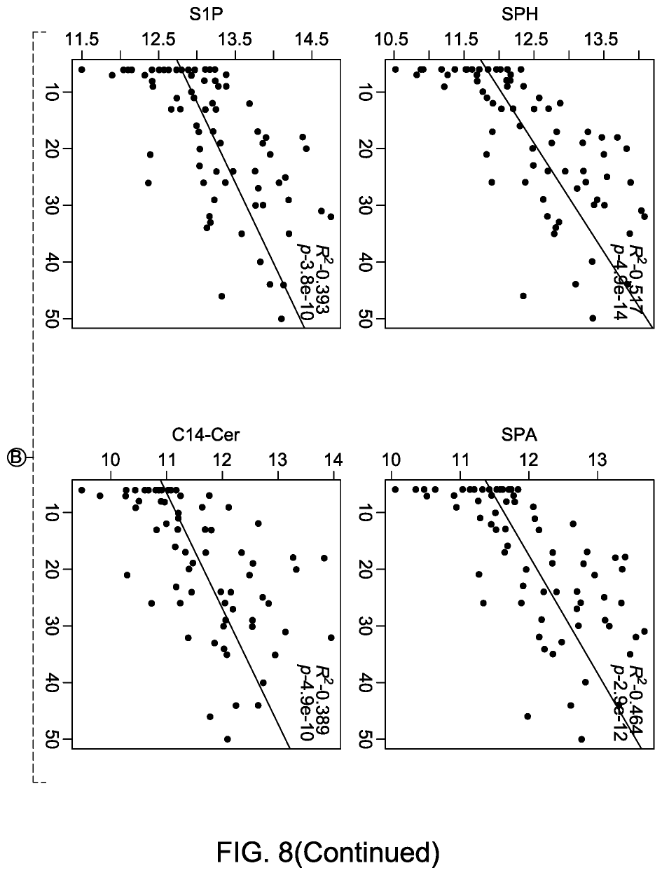

The median concentration of EVs in healthy controls, heavy drinkers, ESLD, decompensated AC, and AH subjects was 4.38×10¹⁰, 1.28×10¹¹, 3.06×10¹⁰, 6.29×10¹⁰, 9.2×10¹⁰, and 5.38×10¹¹ particles/mL respectively (Table 2). Plasma EV concentrations were significantly higher among AH subjects than healthy controls (p<0.0001), heavy drinkers (p=0.0001), ESLD subjects (p<0.0001), and decompensated AC (p<0.0001) (FIG. 1). Moreover, the median EV counts were significantly higher in subjects with severe AH when compared with mild/moderate AH subjects (p=0.0041) (FIG. 1). EV counts correlated well with traditional clinical risk stratifiers, such as the MELD and CTP scores (FIGS. 7-8). Interestingly, plasma EV concentrations were also significantly higher in heavy drinkers (median=1.28×10¹¹ EVs/mL) when compared with healthy controls (median=4.38×10¹⁰; p=0.003). The mean sizes of isolated EVs were 124.3 (healthy controls), 136.6 (heavy drinkers), 126.3 (ESLD), 131 (decompensated AC), and 127.4 (AH) nm and all within small EV size range of 50-150 nm (FIG. 2). The isolated and enriched small EVs expressed EV-specific protein markers cluster of differentiation 81 (CD81; transmembrane protein) and tumor susceptibility gene 101 (TSG101; cytosolic protein recovered in EVs) (FIG. 2). Thus, EV counts are significantly higher in AH subjects when compared to ESLD and decompensated AC subjects.

### Threshold Detection and Validation of Extracellular Vesicle Count for the Diagnosis of Alcoholic Hepatitis

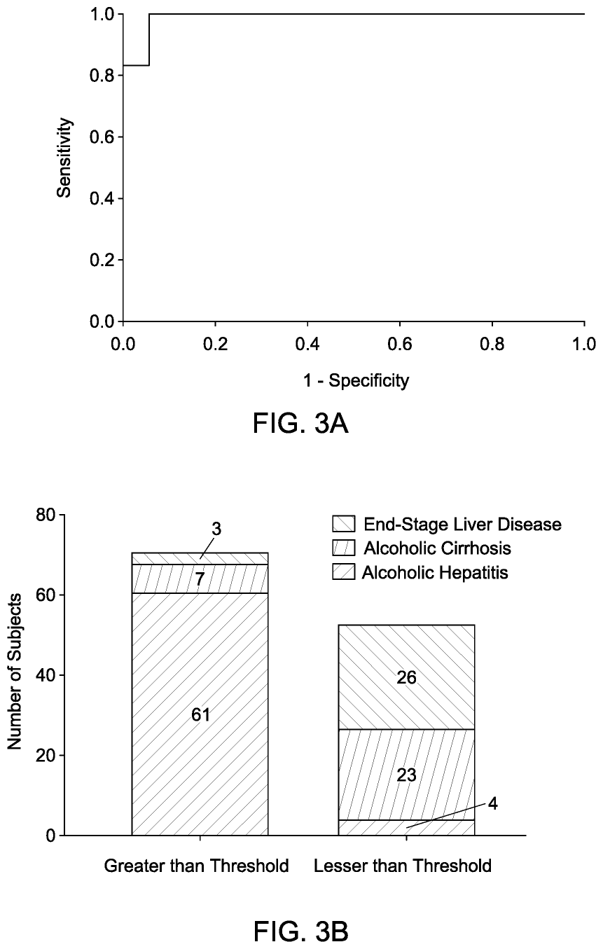

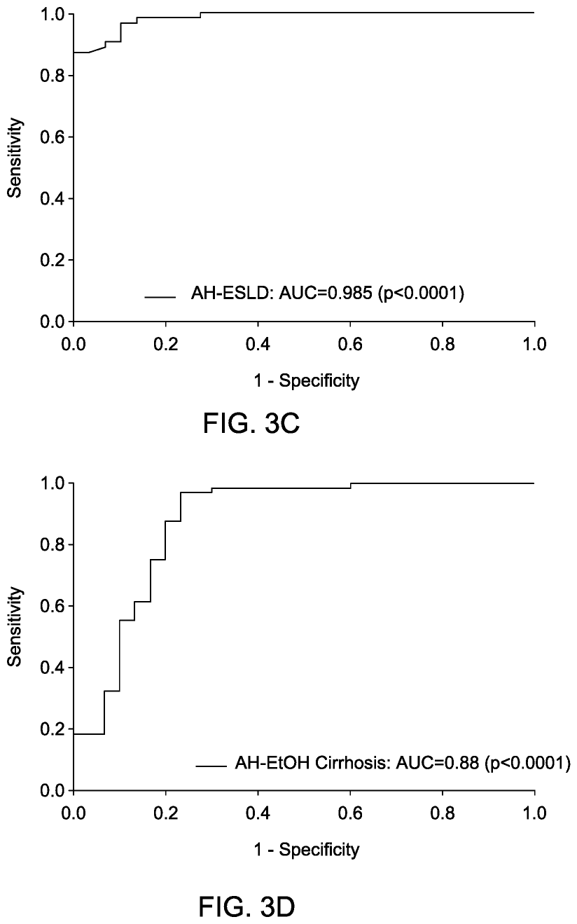

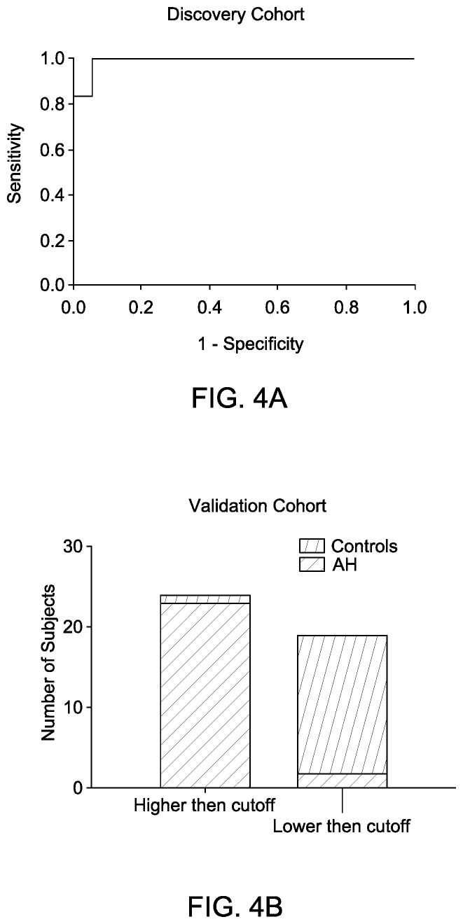

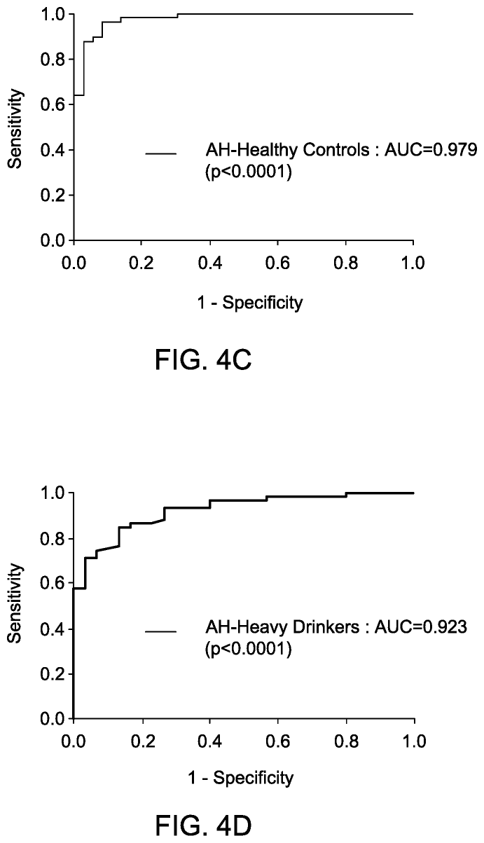

With the aim of establishing a diagnostic threshold, healthy controls and AH subjects from the TREAT consortium were randomly divided in half to establish a discovery cohort. First an ROC curve was generated for the diagnosis of AH vs. healthy controls and the AUROC curve value was 0.99 (95% CI 0.97 to 1.00; p<0.0001) (FIG. 3). A threshold of 1.56×10¹¹ EVs/mL was determined using Youden's index to differentiate AH subjects from healthy controls with a sensitivity of 1.00 (95% CI 0.81 to 1.00) and a specificity of 0.89 (95% CI 0.65 to 0.99). The performance of this diagnostic threshold to diagnose AH was tested in a validation cohort. The validation cohort consisted of 25 AH subjects and 18 healthy controls from TREAT consortium and UPMC. This diagnostic threshold of 1.56×10¹¹ EVs/mL yielded a significantly (p<0.0001) high sensitivity of 0.92 (95% CI 0.75, 0.99) and specificity of 0.94 (95% CI 0.74, 1.00) for distinguishing subjects with AH from healthy controls (FIG. 4, Table 3).

The ability of this diagnostic threshold to differentiate AH from heavy drinkers and disease controls with ESLD and decompensated AC. All of these analyses were performed in complete subject cohorts. This diagnostic threshold was able to differentiate AH subjects from heavy drinkers with a sensitivity of 0.93 (95% CI 0.84, 0.98) and a specificity of 0.73 (95% CI 0.54, 0.88) and an AUROC curve of 0.92 (95% CI 0.87, 0.98; p<0.0001) (FIG. 4, Table 3). Furthermore, this diagnostic threshold was able to differentiate AH from ESLD subjects with a sensitivity of 0.95 (95% CI 0.84 to 0.99) and specificity of 0.90 (95% CI 0.73 to 0.98), and an AUROC curve of 0.99 (95% CI 0.97, 1.00; p<0.0001) (FIG. 3, Table 3). Similarly, it was able to differentiate AH from decompensated AC with a sensitivity of 0.94 (95% CI 0.85 to 0.98) and a specificity of 0.77 (95% CI 0.58 to 0.90) based on ROC curve (AUROC: 0.88; p<0.0001) analysis (FIG. 3, Table 3). This initial analysis confirmed that EVs are significantly elevated in AH and could be of clinical utility. Thus the EV concentration diagnostic threshold of 1.56×10¹¹ EVs/mL can diagnose AH from subjects with ESLD and decompensated AC.

TABLE 3
Diagnostic performance of EV count cut-off determined from
discovery AH cohort for diagnosis in other subgroups.

| | Sensitivity | | Specificity | |
|---|---|---|---|---|
| | | 95% CI | | 95% CI |
| AH validation cohort | 0.92 | 0.75, 0.99 | 0.94 | 0.74, 1.00 |
| Heavy Drinkers | 0.93 | 0.84, 0.98 | 0.73 | 0.54, 0.88 |
| End-Stage | 0.94 | 0.85, 0.98 | 0.90 | 0.73, 0.98 |
| Liver Disease | | | | |
| Alcoholic Cirrhosis | 0.94 | 0.85, 0.98 | 0.77 | 0.58, 0.90 |

Predicting Survival with EV Concentration in AH Subjects

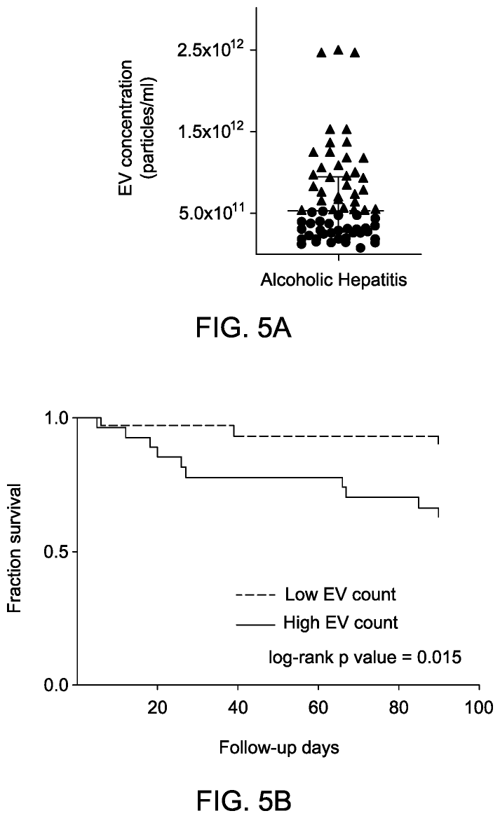

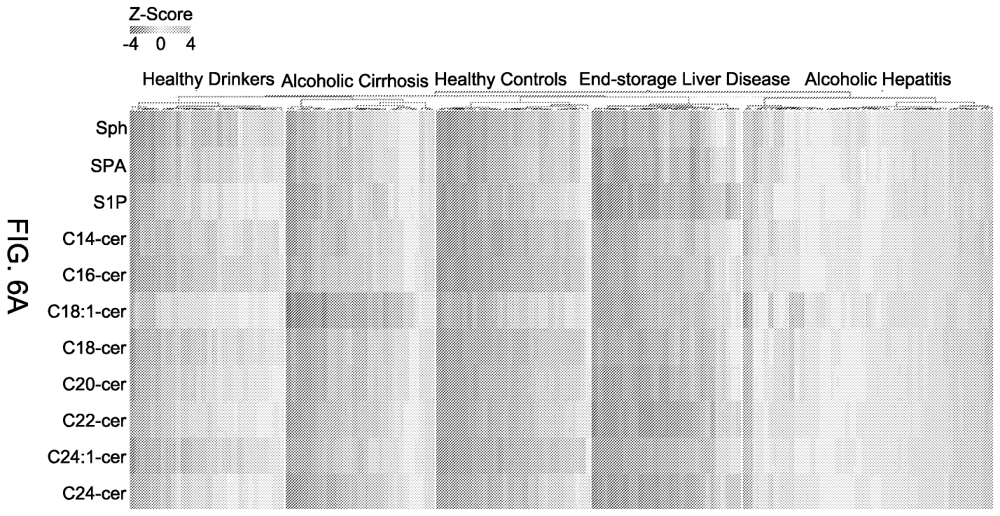

After establishing a diagnostic threshold for AH, the association between EV counts and 90-day survival in AH was investigated. Ninety-day survival follow-up data was available in 57 AH subjects. In order to study this association, these AH subjects were dichotomized into arbitrary low and high EV count groups using median EV count in our cohort (5.38×10¹¹ EVs/mL) (FIG. 5). There were a total of 13 deaths reported at 90-day follow up. 10 deaths were reported in the high EV group vs. 3 deaths in the low EV group. On KM analysis, the mortality risk in subjects with AH at 90-day follow-up was 37.0% in the high EV group and 10% in the low EV group (logrank test p=0.015 and logrank hazard ratio=4.3, 95% CI 1.5 to 13.0) (FIG. 5). Thus, an EV concentration higher than 5.38×10¹¹ EVs/mL predicts a much higher mortality risk in subjects with AH.

### EV Cargo and Plasma Sphingolipids Concentration Analysis

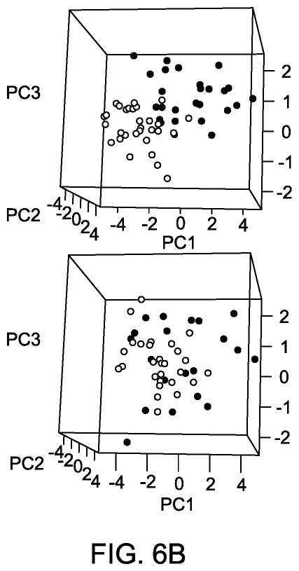

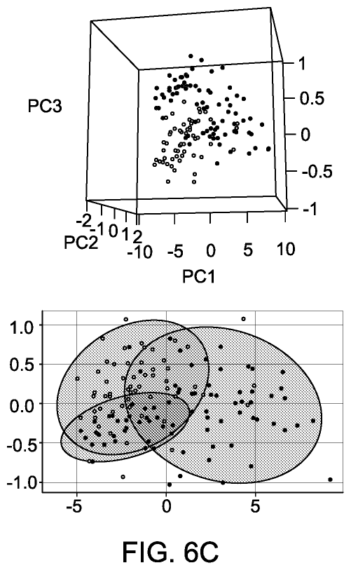

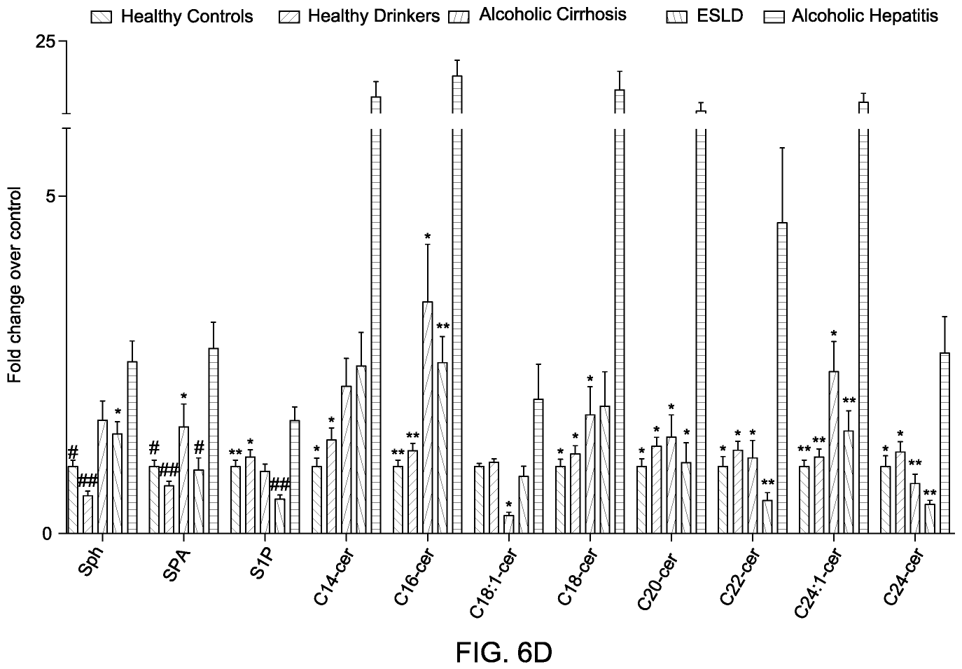

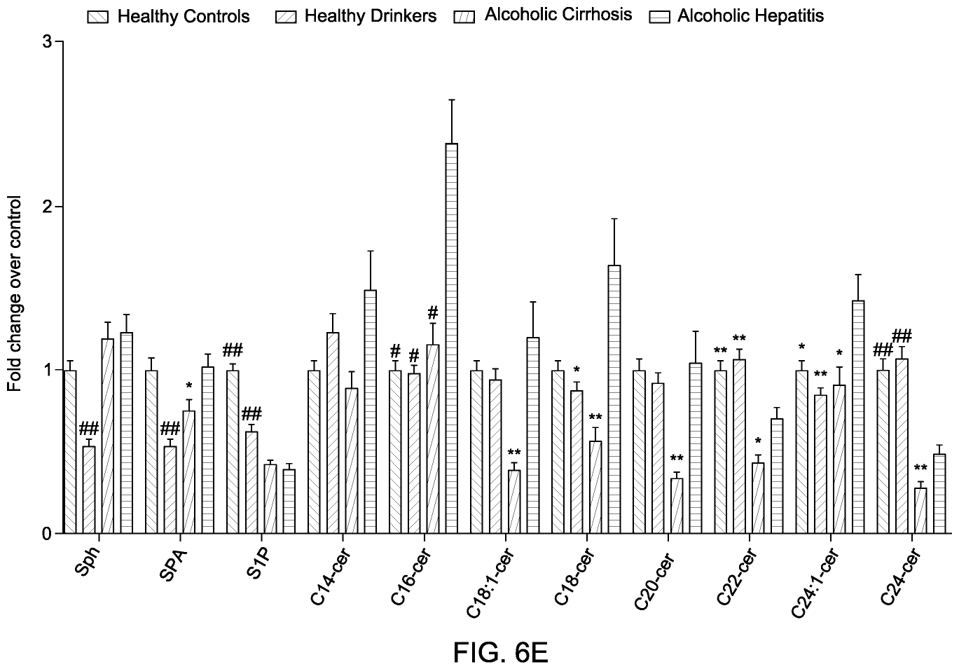

Targeted sphingolipid measurements were performed on isolated EVs and plasma samples from healthy controls, heavy drinkers, ESLD, decompensated AC, and AH subjects. Eleven sphingolipid species were detected from plasma and EV cargo in varying concentrations. These included sphingosine, sphinganine, sphingosine 1-phosphate, C14:0 ceramide, C16:0 ceramide, C18:0 ceramide, C18:1 ceramide, C20:0 ceramide, C22:0 ceramide, C24:0 ceramide, and C24:1 ceramide. The analysis revealed significant differences in sphingolipid cargo content in EVs isolated from AH subjects when compared with healthy controls, heavy drinkers, ESLD, and decompensated AC subjects as evident by the heatmap and principle component analysis (FIG. 6, Table 4). Absolute concentrations of all sphingolipid species detected are provided in Table 4. Of the 11 sphingolipids detected in the EVs, 6 were determined to have statistically significant enrichment in AH subjects when compared with healthy controls, heavy drinkers, ESLD, and decompensated AC subjects (FIG. 6, Table 4). These included C14:0 ceramide, C16:0 ceramide, C18:0 ceramide, C20:0 ceramide, and C24:1 ceramide. Ten sphingolipid species were significantly enriched in AH when compared to healthy controls alone. This sphingolipid signature was found to be unique to EVs and similar trends were not observed in paired plasma samples (FIG. 6, Table 4). To confirm that the increase in EV sphingolipids was not due to a contribution from lipoproteins, the high density lipoprotein (HDL)-specific apoprotein, APOAI, and very low density lipoprotein (VLDL)/low density lipoprotein (LDL)-specific apoprotein, ApoB100 were examined, and comparable expression was found across controls and AH. Thus, the increase in sphingolipids in AH was not due to a co-isolation of HDL, LDL or VLDL lipoproteins (FIG. 2). EV cargo sphingolipid concentrations were found to correlate well with EV counts, whereas plasma sphingolipid concentrations showed no similar trend (Table 5). Thus, a unique, long-chain ceramide-enriched sphingolipid cargo signature was identified in EVs from AH subjects.

TABLE 4
Absolute concentrations of sphingolipid species in all five subgroups.
All values are in nM.

| | | | | | | | End-Stage | | | |
|---|---|---|---|---|---|---|---|---|---|---|
| | Alcoholic | | Healthy | | Heavy | | Liver | | Alcoholic | |
| | Hepatitis | | Controls | | Drinkers | | Disease | | Cirrhosis | |
| | Mean | SD | Mean | SD | Mean | SD | Mean | SD | Mean | SD |
| SPH | 18.21 | 15.05 | 7.14 | 2.99 | 4.03 | 1.95 | 10.53 | 6.41 | 12.02 | 10.30 |
| SPA | 6.75 | 6.57 | 2.45 | 1.15 | 1.74 | 0.8 | 2.31 | 2.03 | 3.86 | 4.65 |
| S1P | 68.10 | 50.02 | 40.31 | 20.22 | 45.96 | 20.8 | 20.58 | 10.76 | 37.05 | 20.05 |
| C14:0 Cer | 4.56 | 9.95 | 0.39 | 0.23 | 0.56 | 0.31 | 1.00 | 0.98 | 0.87 | 0.84 |
| C16:0 Cer | 226.48 | 363.54 | 13.62 | 6.46 | 16.82 | 7.06 | 34.51 | 27.15 | 46.86 | 60.12 |
| C18:1 Cer | 1.50 | 2.61 | 0.74 | 0.09 | 0.80 | 0.11 | 0.64 | 0.50 | 0.20 | 0 15 |
| C18:0 Cer | 67.56 | 155.06 | 5.10 | 2.62 | 6.08 | 2.83 | 9.67 | 13.37 | 9.00 | 10.52 |
| C20:0 Cer | 34.54 | 66.97 | 4.60 | 2.58 | 5.99 | 2.88 | 4.88 | 6.92 | 6.62 | 7.74 |
| C22:0 Cer | 154.38 | 257.16 | 33.51 | 25.58 | 41.87 | 22.09 | 16.44 | 17.20 | 37.79 | 41.71 |
| C24:1 Cer | 326.75 | 552.64 | 33.25 | 17.52 | 37.69 | 19.90 | 50.17 | 51.05 | 79.98 | 76.79 |
| C24:0 Cer | 265.81 | 366.16 | 99.19 | 78.09 | 120.53 | 71.71 | 43.08 | 24.64 | 73.70 | 68.34 |

| | |
|---|---|
| C8:0 Cer | Not Detected |

TABLE 5
Correlation of EV counts with EV cargo and plasma sphingolipids.

| | EV Cargo Sphingolipids | | Plasma Sphingolipids | |
|---|---|---|---|---|
| | Correlation | p-value | Correlation | p-value |
| SPH | 0.79 | <.0001 | 0.41 | 0.03 |
| SPA | 0.69 | <.0001 | 0.47 | <0.01 |
| S1P | 0.67 | <.0001 | 0.12 | 0.53 |
| C14:0 Cer | 0.56 | <0.01 | 0.39 | 0.03 |
| C16:0 Cer | 0.74 | <.0001 | 0.39 | 0.03 |
| C18:1 Cer | 0.70 | <.0001 | 0.31 | 0.10 |
| C18:0 Cer | 0.69 | <.0001 | 0.34 | 0.06 |
| C20:0 Cer | 0.73 | <.0001 | 0.38 | 0.04 |
| C22:0 Cer | 0.76 | <.0001 | 0.35 | 0.06 |
| C24:1 Cer | 0.76 | <.0001 | 0.47 | <0.01 |
| C24:0 Cer | 0.70 | <.0001 | −0.08 | 0.69 |

Correlation of Extracellular Vesicle Sphingolipids with MELD and CTP Scores

An association between specific sphingolipid species and disease severity and mortality was examined. By linear regression, significant positive correlations were found between MELD and log EV counts (r²=0.475, p<0.0001) as well as all 11 detected EV cargo sphingolipids including sphingosine, sphinganine, sphingosine-1 phosphate, C14:0 ceramide, C16:0 ceramide, C18:0 ceramide, C18:1 ceramide, C20:0 ceramide, and C24:1 ceramide (FIGS. 7-8, Table 6-7). A similar significant positive correlation was also observed with CTP score and log EV count (r²=0.441, p<0.0001) as well as all 11 detected EV count sphingolipids (FIGS. 7-8, Table 6-7). By linear regression, models against disease severity determined by both CTP and MELD scores, EV cargo sphingosine, sphinganine, sphingosine 1 phosphate, C14:0 ceramide, C16:0 ceramide, and C24:1 ceramide had significant positive correlations with r²>0.3 and p<0.05. Thus, it was found that EV counts and sphingolipid cargo to correlate positively with MELD and CTP scores.

TABLE 6
Linear Regression Modeling depicting correlation between
EV count-Sphingolipid and MELD score.

| | r² | p-value |
|---|---|---|
| SPH | 0.52 | <0.0001 |
| SPA | 0.46 | <0.0001 |
| S1P | 0.39 | <0.0001 |
| C14:0 Cer | 0.39 | <0.0001 |
| C16:0 Cer | 0.46 | <0.0001 |
| C18:1 Cer | 0.27 | <0.0001 |
| C18:0 Cer | 0.30 | <0.0001 |
| C20:0 Cer | 0.29 | <0.0001 |
| C22:0 Cer | 0.28 | <0.0001 |
| C24:1 Cer | 0.40 | <0.0001 |
| C24:0 Cer | 0.25 | <0.0001 |

TABLE 7
Linear Regression Modeling depicting correlation between
EV count-Sphingolipid and CTP score.

| | r² | p-value |
|---|---|---|
| SPH | 0.52 | <0.0001 |
| SPA | 0.46 | <0.0001 |
| S1P | 0.33 | <0.0001 |
| C14:0 Cer | 0.37 | <0.0001 |
| C16:0 Cer | 0.45 | <0.0001 |
| C18:1 Cer | 0.17 | <0.0001 |
| C18:0 Cer | 0.28 | <0.0001 |
| C20:0 Cer | 0.25 | <0.0001 |
| C22:0 Cer | 0.25 | <0.0001 |
| C24:1 Cer | 0.38 | <0.0001 |
| C24:0 Cer | 0.20 | <0.0001 |

Performance of EV Counts with EV Cargo Sphingolipids

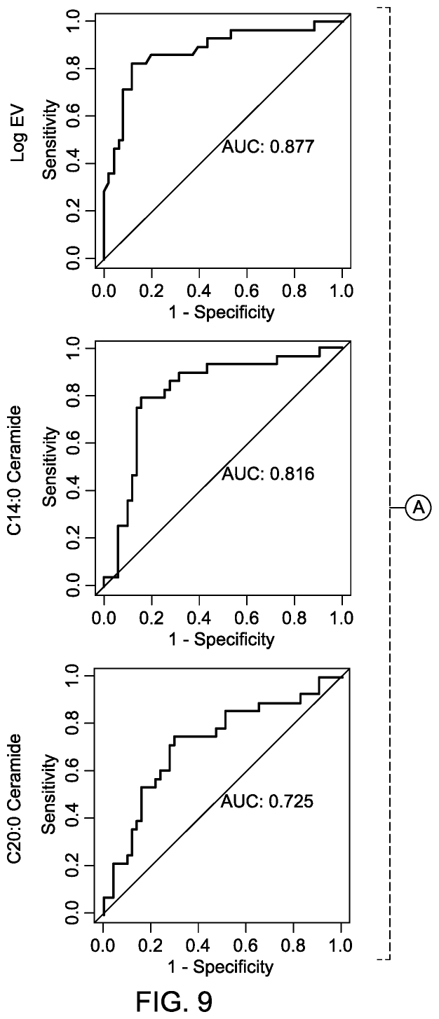

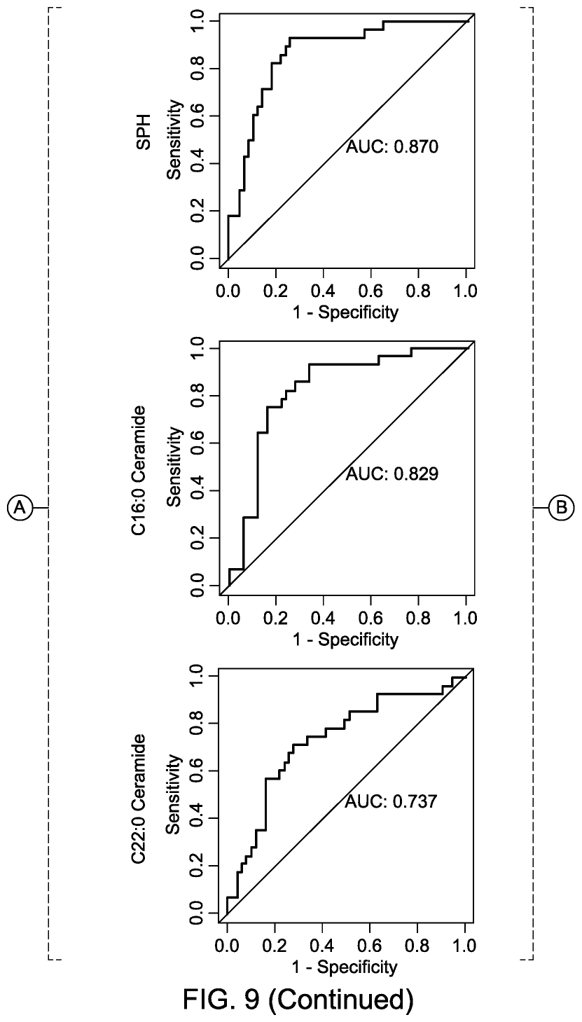

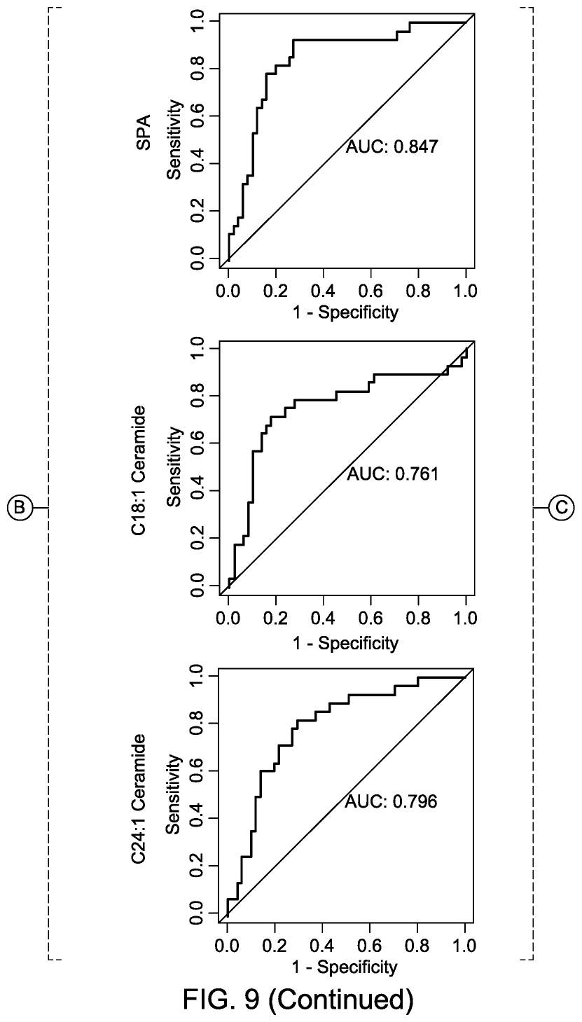

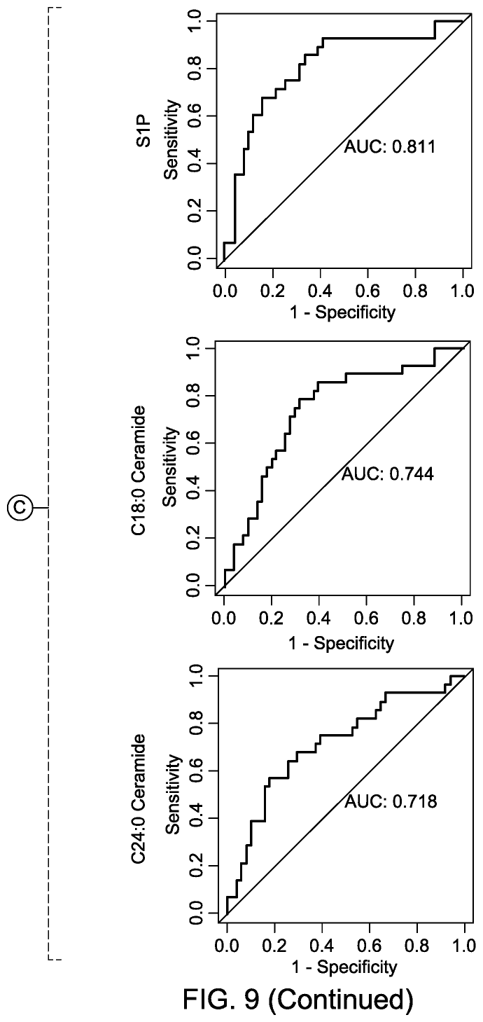

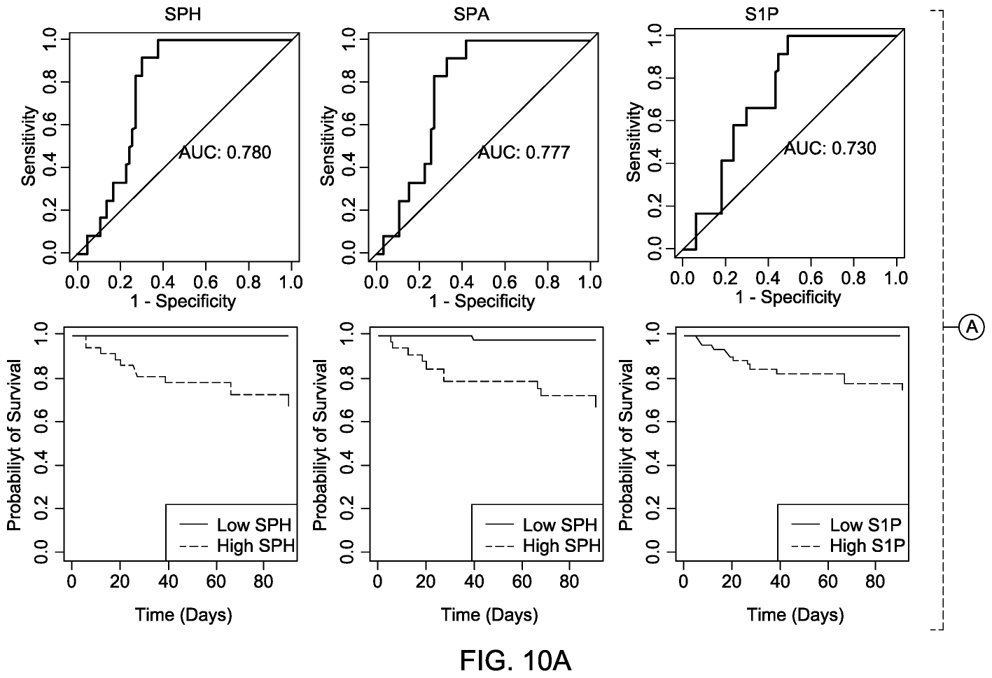

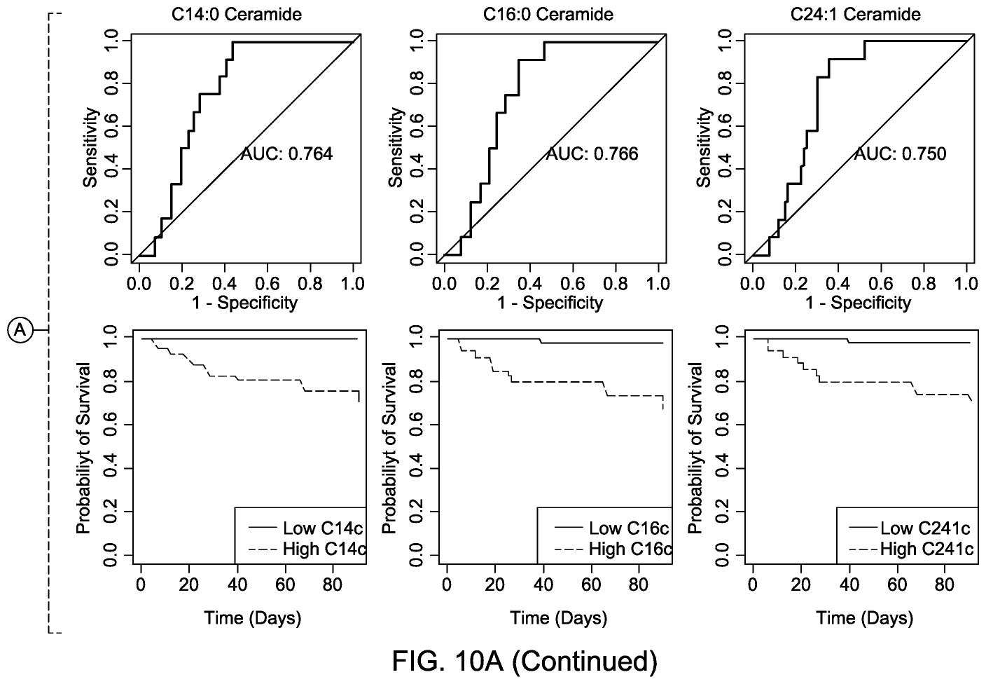

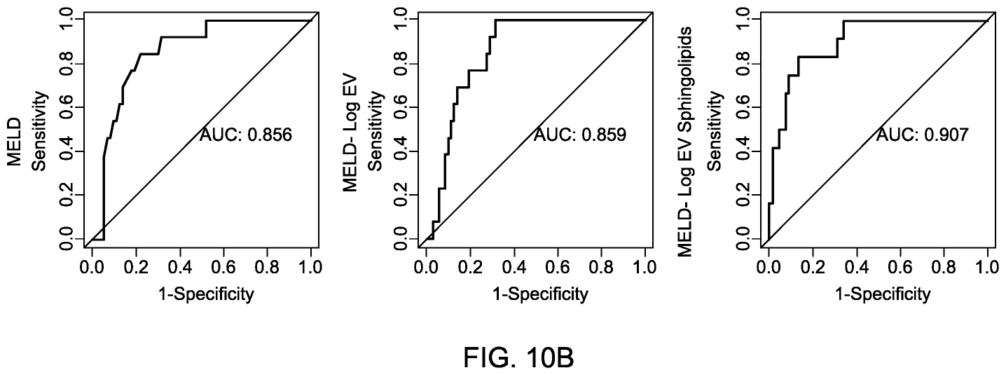

A combinatorial approach was adopted to determine if the interaction of EV sphingolipid content and EV count can predict mortality. Univariate Cox modeling was performed to ascertain risk association with 90-day mortality for each individual sphingolipid as well as EV x sphingolipid (Table 8). MELD score, CTP score, and total bilirubin were all associated with increased risk of mortality. In addition, 6 EV cargo sphingolipids sphingosine, sphinganine, sphingosine 1-phosphate, C14:0 ceramide, C16:0 ceramide, and C24:1 ceramide were predictive of higher mortality risk at 90-day follow up. When these significant risk factors for mortality were analyzed in a multivariate analysis, none attained statistical significance, likely due to limited sample size. The association of EVs and sphingolipids with disease severity (as determined by MELD and CTP scores) and mortality was then assessed and plotted in the form of ROC curves. Youden's index was used to determine optimal cutoffs for each individual sphingolipid when interacting with EV count as EV x sphingolipid (FIGS. 9-10, Tables 9-11). Multiple sphingolipids had good discriminatory power in determining disease severity based on these cutoffs, but under-performed compared to the MELD score or EV count alone. KM curves were plotted for each potential biomarker based on these generated cutoffs, and their ability to prognosticate AH was determined (FIGS. 9-10, Tables 9-11). Performance of EV x Sphingolipid together had enhanced performance when considering disease severity or mortality as outcome (FIGS. 9-10, Tables 9-11). The same 6 sphingolipids identified to have positive hazards ratios in the Cox's Univariate analysis were also found to prognosticate the mortality of AH subjects in KM curves with significance. An ROC curve was generated combining MELD score, log EV count and these 6 sphingolipid populations to generate an E-MELD score and determine association with 90-day mortality. The AUROC was determined to be 0.91. Thus, even though discriminatory capacity for MELD-log EV alone (AUC=0.86) was similar to MELD (AUC=0.86), E-MELD performed better (AUC=0.91) in predicting mortality (FIGS. 9-10, Tables 9-11).

TABLE 8
Univariate Cox Regression analysis of 90-day mortality.

| | Hazard | 95% | 95% | |
|---|---|---|---|---|
| | Ratio | CI-Low | CI-High | p-value |
| Age | 1.033 | 0.978 | 1.092 | 0.239 |
| Albumin | 0.456 | 0.175 | 1.191 | 0.109 |
| T. Bilirubin | 1.072 | 1.039 | 1.105 | <0.0001 |
| Creatinine | 1.260 | 0.922 | 1.721 | 0.147 |
| ALT | 1.000 | 0.992 | 1.008 | 0.971 |
| AST | 1.003 | 0.998 | 1.008 | 0.259 |
| ALP | 1.004 | 1.001 | 1.007 | 0.003 |
| T. Protein | 0.457 | 0.212 | 0.985 | 0.046 |
| BMI | 1.084 | 0.987 | 1.191 | 0.091 |
| MELD | 1.078 | 1.037 | 1.120 | 0.0002 |
| SPH | 2.668 | 1.301 | 5.471 | 0.007 |
| SPA | 2.644 | 1.281 | 5.456 | 0.009 |
| S1P | 2.440 | 1.052 | 5.659 | 0.038 |
| C14:0 Cer | 1.899 | 1.094 | 3.296 | 0.023 |
| C16:0 Cer | 1.970 | 1.153 | 3.366 | 0.013 |
| C18:1 Cer | 1.836 | 0.885 | 3.806 | 0.103 |
| C18:0 Cer | 1.573 | 0.934 | 2.650 | 0.089 |
| C20:0 Cer | 1.619 | 0.912 | 2.874 | 0.100 |
| C22:0 Cer | 1.724 | 0.926 | 3.209 | 0.086 |
| C24:1 Cer | 1.919 | 1.091 | 3.374 | 0.024 |
| C24:0 Cer | 1.798 | 0.901 | 3.589 | 0.096 |

TABLE 9
ROC metrics depicting ability of parameters to predict
90-day mortality, as well as Youden’s index determined
cut-off point for plotting KM curves.

| | AUC | p-value | Cut-Off Point |
|---|---|---|---|
| MELD | 0.859 | 0.01 | 24 |
| Log EV | 0.805 | 0.0031 | 11.48555 |
| SPH | 0.780 | 0.0080 | 12.37172 |
| SPA | 0.777 | 0.0091 | 12.14333 |
| S1P | 0.730 | 0.0369 | 13.08564 |
| C14:0 Cer | 0.764 | 0.0256 | 11.23493 |
| C16:0 Cer | 0.766 | 0.0147 | 13.37651 |
| C18:1 Cer | 0.658 | 0.1107 | Not Significant |
| C18:0 Cer | 0.711 | 0.0878 | Not Significant |
| C20:0 Cer | 0.688 | 0.1011 | Not Significant |
| C22:0 Cer | 0.701 | 0.0848 | Not Significant |
| C24:1 Cer | 0.750 | 0.0255 | 13.51354 |
| C24:0 Cer | 0.693 | 0.0940 | Not Significant |

TABLE 10
P-values for KM Curve depicting 90-day mortality.

| | p-value |
|---|---|
| SPH | <0.0001 |
| SPA | 0.0001 |
| S1P | 0.0013 |
| C14:0 Cer | 0.0003 |
| C16:0 Cer | 0.0001 |
| C24:1 Cer | 0.0003 |

TABLE 11
ROC metrics depicting ability of parameters to predict disease
severity (determined using a MELD score cut-off of 20).

| | AUC | p-value |
|---|---|---|
| Log EV | 0.88 | <0.0001 |
| SPH | 0.87 | <0.0001 |
| SPA | 0.85 | <0.0001 |
| S1P | 0.81 | <0.0001 |
| C14:0 Cer | 0.82 | 0.0001 |
| C16:0 Cer | 0.83 | <0.0001 |
| C18:1 Cer | 0.76 | 0.0009 |
| C18:0 Cer | 0.74 | 0.0015 |
| C20:0 Cer | 0.72 | 0.0018 |
| C22:0 Cer | 0.74 | 0.0014 |
| C24:1 Cer | 0.80 | 0.0001 |
| C24:0 Cer | 0.72 | 0.0024 |

Together, these results demonstrate that a liquid biopsy-based EV biomarker can be used not only to diagnose AH, but also assess the risk of 90-day mortality from the AH.

### Example 2: The C16 Ceramide and S1P Content of Circulating EVs as a Biomarker in AH

### EV Sphingolipids are Increased in AH

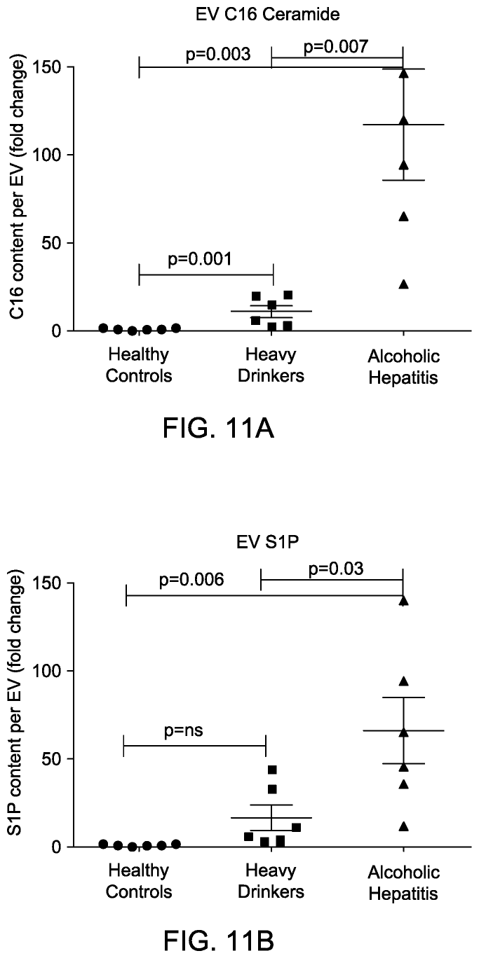

Quantification of the sphingolipidomic content of plasma EVs was performed using tandem mass spectroscopy (LC-MS/MS). An increase in EV C16 ceramide was seen in HD compared to normal controls, and it was further significantly increased in AH EVs (FIG. 11A). Similarly EV S1P was significantly increased in AH compared with HD (FIG. 11B). In contrast, an increase in either C16 ceramide or S1P was not seen in matched plasma samples, suggesting that the enrichment of these two sphingolipids in EVs is likely linked to the ongoing liver injury in AH and not due to a non-specific increase in circulating levels of C16 ceramide or S1P.

EV C16 Ceramide and S1P Content Correlate with Parameters of Liver Injury and MELD

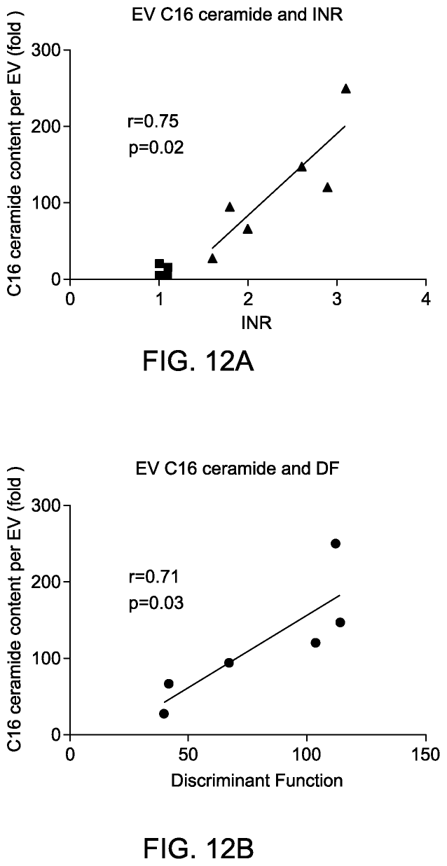

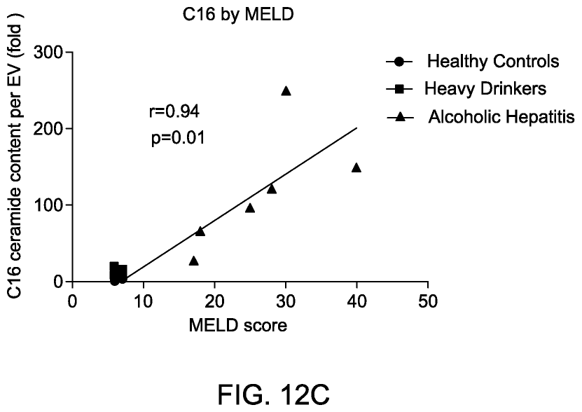

Diagnosis of AH is based on the presence of markers of liver injury on routine biochemical testing of liver function in conjunction with a history of heavy alcohol consumption. The international normalized ratio (INR) is particularly important among these tests as it is a direct measure of loss of normal hepatocellular synthetic function due to acute hepatocellular injury. The importance of INR is highlighted in its incorporation in almost all prognostic scores for AH, including Maddrey's discriminant function (DF), age-bilirubin-INR-creatinine (ABIC), the Glasgow AH score and the MELD, wherein INR is the heaviest weighted component. Therefore, it was reasoned that for EV C16 ceramide and S1P content to serve as diagnostic tools in AH, they would be predicted to match INR, and bilirubin and multiparametric scores such as the DF and MELD. Therefore, the correlation of EV C16 ceramide content with INR, DF and MELD was compared by linear logistic regression (FIG. 12A-C). It was found that EV C16 ceramide content correlated well with INR, DF and MELD (FIG. 12); however, it did not correlate well with creatinine, which, being a renal marker, does not reflect hepatocellular injury. EV S1P demonstrated similar correlations with INR and MELD.

Therefore, in summary, these data demonstrate:
An increase in circulating EVs in AH;
Significant increase in EV 16 ceramide and S1P content in AH;
EV 16 ceramide and S1P content correlate with parameters of hepatocellular injury; and
EV 16 ceramide and S1P content correlate with MELD such that EV C16 ceramide and S1P can diagnose AH in a well-characterized derivation cohort with matched heavy drinking controls, followed by validation in an independent multi-site cohort.

### Methods

### Study Design

A first cohort is comprised of well-characterized AH subjects with matched heavy drinking controls. De-identified plasma samples from these subjects are utilized. Normal human plasma samples (healthy controls) are sex, age, and race matched. Plasma samples collected at study entry are referred to as Day 0 (D0) samples. EVs are isolated using standardized differential ultracentrifugation (UTC) and their sphingolipids measured. An EV C16 ceramide and S1P cutoff value and a statistical model is established to predict the diagnosis of AH. Mortality prediction using D0 EV C16 and S1P is a secondary endpoint.

A second cohort is derived from plasma samples from AH subjects and HD controls archived under a multi-site consortium grant, thus forming an independent multi-site validation cohort. These samples are handled, EVs isolated, and sphingolipidomics performed in a blinded manner. Using the EV C16 ceramide and EV S1P cutoffs and model established with the derivation cohort, the diagnostic performance of these EV sphingolipid-based biomarkers are assessed. Mortality prediction is a secondary endpoint in this cohort as well.

### Plasma EV Sphingolipidomics

Plasma is collected from EDTA anticoagulated whole blood, depleted of platelets by centrifugation and stored at −80° C. until isolation of EVs by UTC. Aliquots of plasma totaling 1 mL each are thawed on ice. Following this, 800 μL of plasma is utilized to isolate total circulating EVs utilizing UTC as described elsewhere (see, e.g., Kakazu et al., J Lipid Res.; 57(2):233-45 (2016); and Altamirano et al., Gastroenterology.; 146(5):1231-9 el-6 (2014)). Matched 25 μL plasma aliquots are reserved for the measurement of plasma sphingolipids and placed at −80° C. Isolated EVs are resuspended in 100 μL of sterile PBS. A 5 μL aliquot of total EVs isolated by UTC is utilized for EV quantification using nanoparticle tracking analysis (NTA), and the remainder stored at −80° C. for sphingolipid measurements. Sphingolipids are extracted from EVs suspended in PBS or plasma after the addition of internal standards and sonication. The extracts are measured against a standard curve on the Thermo TSQ Quantum Ultra mass spectrometer (Thermo Scientific, West Palm Beach, Fla.) coupled with a Waters Acquity UPLC system (Waters, Milford, Mass.), as described elsewhere (see, e.g., Blachnio-Zabielska et al., Rapid Commun Mass Spectrom; 26(9):1134-40 (2012)). 12 sphingolipid species are in EVs: sphinganine, sphingosine, sphingosine 1-phosphate, C8-ceramide, C14-ceramide, C16-ceramide, C18:1-ceramide, C18-ceramide, C20-ceramide, C22-ceramide, C24:1-ceramide, and C24-ceramide.

### Example 3: EV Sphingolipids Predict Survival and Decrease with Cytoprotective Therapy

EV sphingolipids correlate with mortality, and EV sphingolipid-induced proinflammatory macrophage effector responses decrease after IL-22 treatment.

EV C16 Ceramide and S1P Content Correlate with MELD Score and Improve Over Time

The MELD score, an index based on bilirubin, INR, and creatinine, accurately predicts mortality in AH subjects and correlates with the severity of illness. The correlation of EV C16 ceramide content (FIG. 12C) and EV S1P content with MELD was compared by linear logistic regression. There was high correlation between both C16 ceramide content and MELD and S1P content and MELD.

### IL-22 Inhibits EV Release in Response to Alcohol In Vitro

IL-22 is being investigated as a potential therapy in AH due to its hepatoprotective effects. To investigate if IL-22 can inhibit ethanol-induced EV release, HepG2Cyp2E1 (human hepatoma cells that can metabolize ethanol due to the enforced expression of cytochrome P450 2E1) cells were pretreated with IL-22 prior to the addition of ethanol. Subsequently, EVs were isolated from cell culture supernatants by UTC and quantified with NTA. Similarly, IL-22 reduced EV counts in AH patients after treatment. These results suggest that ethanol-induced EV release was significantly inhibited with IL-22 treatment.

### EV C16 and S1P Improve Over Time

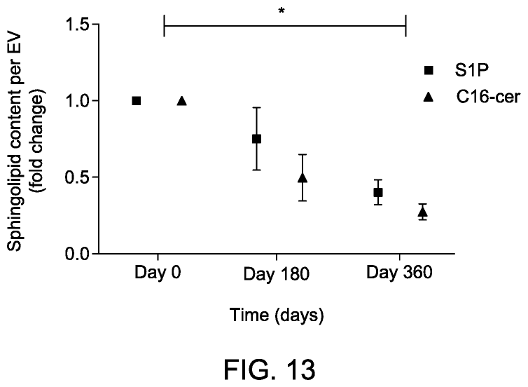

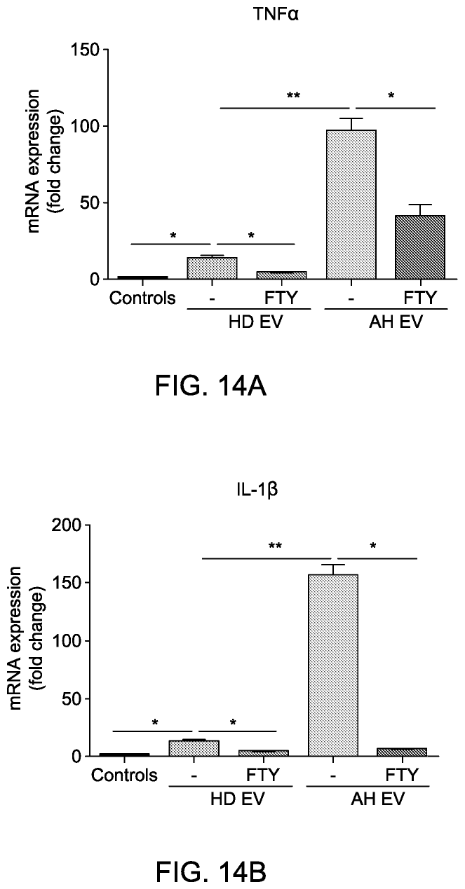

EV C16 and S1P content improve over time in AH patients who did not have adverse outcomes (no re-hospitalization or mortality) and continued to follow-up in the outpatient clinic (FIG. 13). A reduction of EV C16 ceramide content of 25% was observed at day 180 and 60% at day 360 of follow-up; similarly, EV S1P content decreased by 51% at day 180 and 73% by day 360 of follow-up. These data suggest that a hepatoprotective cytokine that hastens recovery might lead to a decrease in EV sphingolipids in vivo.

### Macrophage Proinflammatory Activation is Synergistically Increased by EV S1P

Analysis of EV lipid cargo demonstrates that AH-EVs were several-fold enriched in S1P (FIG. 11B and FIG. 6D); however, the signaling effects and the relevance of this sphingolipid cargo in EVs is poorly studied. PBMC-derived macrophages were treated with EVs derived from HD or AH subjects. AH EVs activated greater proinflammatory signaling in macrophages than HD EVs; this was S1P-dependent as it was significantly inhibited by pharmacologic inhibition of S1P receptors by FTY720.

Therefore, in summary, these data demonstrate:
Robust correlation between EV 16 ceramide or S1P with MELD score;
IL-22 treatment inhibits EV release;
Reduction in EV C16 ceramide and S1P occur with AH resolution; and
EV S1P activates proinflammatory macrophage effector responses.

### Methods

### Study Design

Day 0 (D0) plasma from subjects is utilized to isolate EVs by UTC followed by EV quantification and the measurement of EV sphingolipids. The primary endpoint is to determine the performance of EV 16 ceramide content and EV S1P content in predicting Day 90 mortality. EV C16 ceramide and EV S1P cutoffs are established that predict survival and establish AUROCs for each sphingolipid in predicting survival.

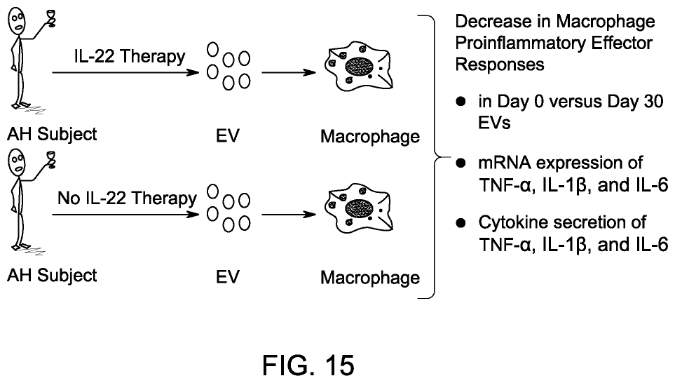

Subjects are treated with IL-22 or placebo. Cytoprotective effects of IL-22 can reduce EV release and EV S1P content. Therefore, a mechanistic basis for hepatocyte-derived EV-induced activation of proinflammatory macrophage effector responses is assessed. EVs are isolated from D0 and Day 30 (D30) samples from IL-22-treated and placebo-treated subjects. Macrophages are derived from PBMC using standardized protocols. Briefly, a density gradient method is utilized for the isolation of monocytes which are ultimately differentiated into macrophages from either the IL-22-treated group or placebo-treated group (FIG. 15).

To study the EV-macrophage cross-talk, AH-EVs are applied to macrophages from both groups. The inflammatory gene expression profile of macrophages is assessed by mRNA expression of the cytokines TNFα, IL-1β, and IL-6. Macrophage cytokine secretion is assessed by performing ELISA for TNFα, IL-1β, and IL-6. Experiments are performed in a cross-over fashion, i.e., EVs from IL-22 treated subjects are applied to control macrophages and IL-22 macrophages, and vice versa. D30 macrophage responses are compared to D0 responses to determine kinetic changes in EV-induced macrophage inflammatory responses.

To demonstrate specificity for EV S1P cargo in activating macrophage effector responses, the above experiments are conducted with pharmacologic inhibitors of S1PR1 (FTY720, W146) to block the S1P receptors on macrophages, and isolated EVs are treated with recombinant active S1P lyase to degrade the bioactive S1P (SRP0191, Sigma Aldrich) before applying EVs to macrophages.

### Example 4: Detecting Hepatocyte-Derived EVs

### ASGR2 is Expressed on Hepatocyte-Derived EVs

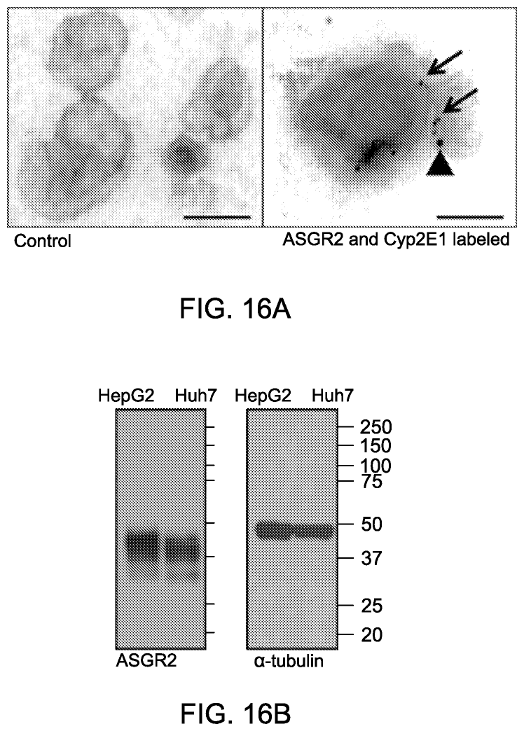

Immuno-gold labeled transmission electron microscopy was used to confirm that ASGR is present on hepatocyte-derived EVs (FIG. 16A, larger gold particle size, arrowhead points to ASGR2). To confirm that these were hepatocyte-derived EVs; Cyp2E1 was also detected on these EVs (FIG. 16A, smaller gold particle size, arrow). For ASGR1 affinity capture, an antibody (BD Pharmingen) that recognizes the extracellular domain was selected. For ASGR2 an antibody raised against amino acids 200-300 was selected, thus recognizing the extracellular domain of ASGR2 (Abcam). The ASGR1 antibody detected several non-specific bands, whereas, the ASGR2 antibody was highly specific by western blotting (FIG. 16B). Only bands at the predicted size were detected, without any non-specific bands.

### ASGR2 can be Utilized for Isolation of Hepatocyte-Derived EVs

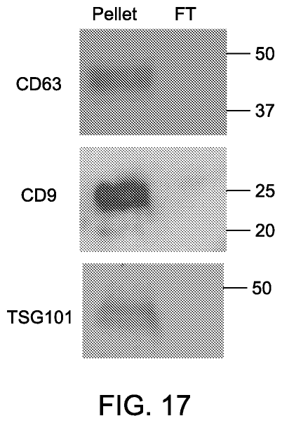

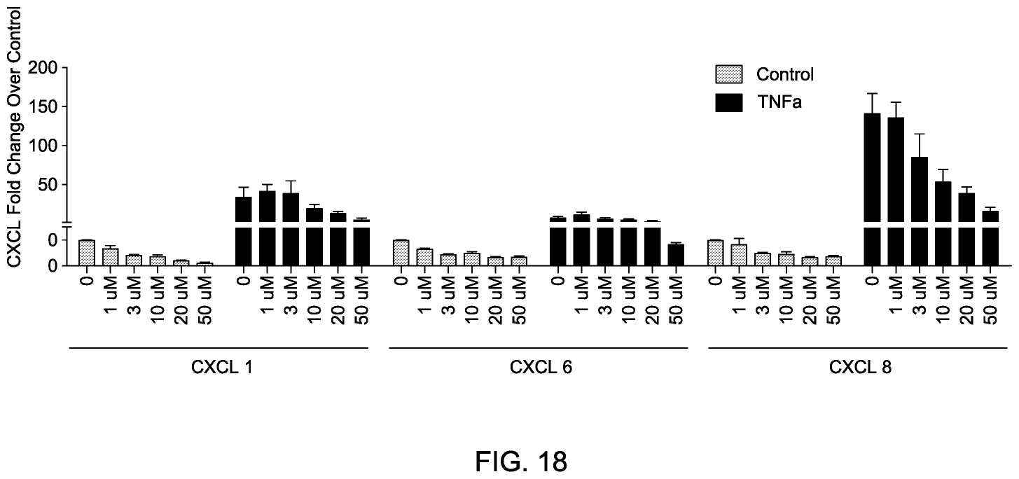

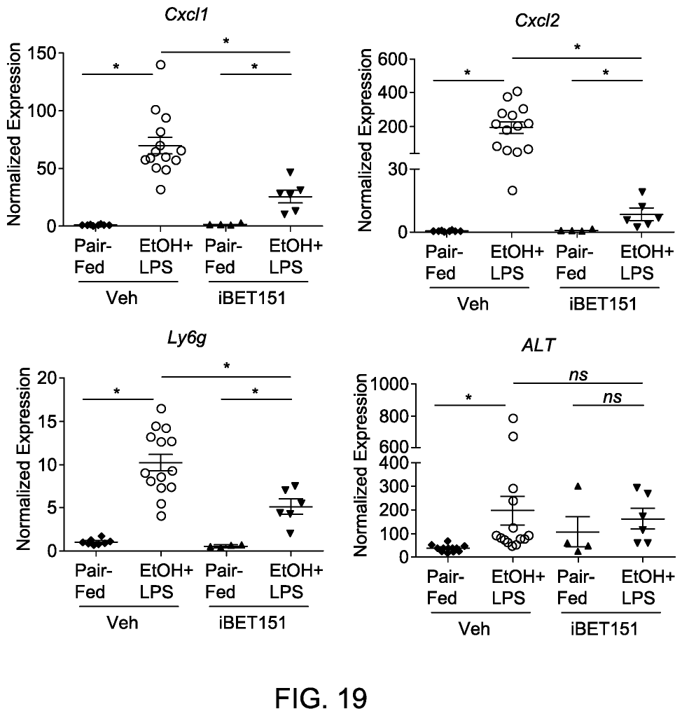

An immune affinity-based EV capture method offers several advantages including the isolation of cell-specific EVs without the need for pre-enrichment of EVs. Furthermore, systematic analyses have demonstrated that immune affinity-based EV capture is more efficient than UTC. Utilizing an anti-ASGR2 antibody coupled to magnetic beads, a method was developed for the immune affinity-based magnetic capture (IAMC) of ASGR2 expressing EVs from plasma. EVs were isolated from 800 μL of plasma. The EV-depleted plasma was further subjected to UTC to isolate remaining EVs. The enrichment of standardized EV markers, CD63, CD9, and TSG101 in IAMC-captured EVs was demonstrated by western blotting (FIG. 17).

### Example 5: Super Enhancer Regulation of Cytokine-Induced Chemokine Production in Alcoholic Hepatitis

This Example describes a role for the super enhancer-regulated pathway in AH pathogenesis, and investigates the therapeutic potential of BET inhibition in AH treatment.

### Materials and Methods

### Cell Culture and TNFα Stimulation

Primary human LSECs were purchased from ScienCell (Cat #5000) and cultured using standard cell culture techniques. Where appropriate, LSECs underwent TNFα (Peprotech, 300-01A) stimulation at 20 ng/mL. For experiments with BRD4 inhibitor, iBET151 (Cayman Chemical 11181), low passage LSECs were plated at 70% confluency and cultured overnight. Next, LSECs were serum starved for 2 hours in low serum medium (0.5% FBS in basal endothelial medium (Lonza CC-3121)). Inhibitors were added at concentrations indicated to low-serum medium, and cells were incubated in inhibitor containing medium for 2 hours. After inhibitor treatment, medium change was performed with TNFα (Peprotech 300-01A) at 20 ng/mL or control low-serum medium for 90 minutes incubation. LSECs were then collected for analysis. In selected experiments, cell supernatant was collected and enzyme-linked immunosorbent assay (ELISA) performed to assess concentration of secreted CXCL1. RNA was extracted and qPCR performed for expression of CXCLs.

### ChIP

LSECs were treated with appropriate conditions as outlined separately, and underwent CHIP according to Millipore High-Sens ChIP kit (Millipore MAGNA0025) manufacture protocols. Briefly, cells were crosslinked with formaldehyde (1% final concentration) followed by glycine treatment (100 mM) for 5 minutes each. Cells were washed, collected, and pelleted with centrifugation, and were then lysed with cell lysis buffer. Cells were repelleted and underwent nuclear lysis with provided nuclear lysis buffer, and DNA was sheared with ultrasonification. Soluble chromatin was aliquoted and immunoprecipitated with magnetic beads with antibodies for BRD4 (Abcam ab128874), NF-κB (Cell signaling 8242S), or H3K9me3 (Abcam ab8898) with appropriate isotype controls. Immunoprecipitated beads were collected and processed according to manufacture protocol. Real-time PCR was performed in purified ChIP and input DNAs at target loci, and enrichment was compared with isotype control IgG.

In vitro Pharmacologic Inhibitor Assays

LSECs were treated with Celastrol (Sigma C0869) or iBET151 (Cayman 11181).

Alcohol Feeding with LPS Model with BRD4 Inhibitor iBET151

WT C57BL/6 mice (10-12 weeks) were purchased from Envigo Laboratories. Mice were subjected to chronic alcohol feeding to induce alcohol-induced liver injury with modification to the NIAAA model (see, e.g., Kong et al., Sci. Rep., 7(1):9292 (2017)). Briefly, mice were acclimated to liquid diet for 5 days, and were fed either 5% alcohol containing liquid diet or isocaloric pair-fed control diet. On Day 11, LPS (Invivogen tlrl-eblps) was intraperitoneally (IP) injected at 4 mg/kg to alcohol-fed mice. PBS of the same volume was given to pair-fed mice. All mice were sacrificed 8 hours later. A subset of mice, concurrent with alcohol or pair-feeding, was given injection of iBET151 daily. Mice were injected IP with iBET151 (6 mg/kg) in 10% Kleptose 2% DMSO solution or equal volume of carrier solution. On Day 11, drug or vehicle was administered 1 hour before LPS injection.

### Statistical Analysis

Means are expressed as means±standard deviation. Statistical analysis was conducted using GraphPad PRISM (La Jolla, USA) and R statistical software. Comparisons between three groups or more were conducted using one-way ANOVA with Dunnet's or Tukey's post-test for multiple comparisons using GraphPad PRISM. Comparisons with two different conditions were performed with two-way ANOVA with Sidak's or Tukey's post-test for multiple comparisons. A comparison of two groups was performed using the Student's t test. P values are as follows: ****p≤0.0001, ***p≤0.001, **p≤0.01, and *p≤0.05.

### Results

### Epigenetic Suppression of the CXCL Super Enhancer and CXCL1 Promoter Sites Modulate Chemokine Gene Expression

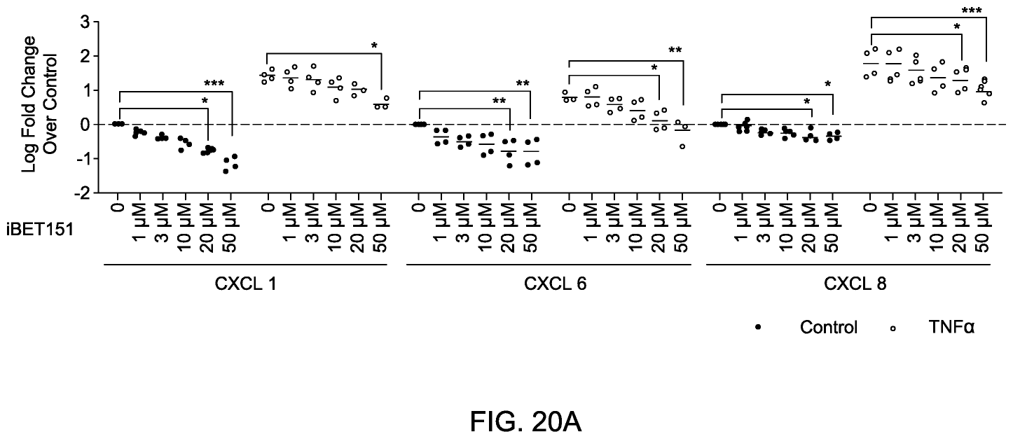

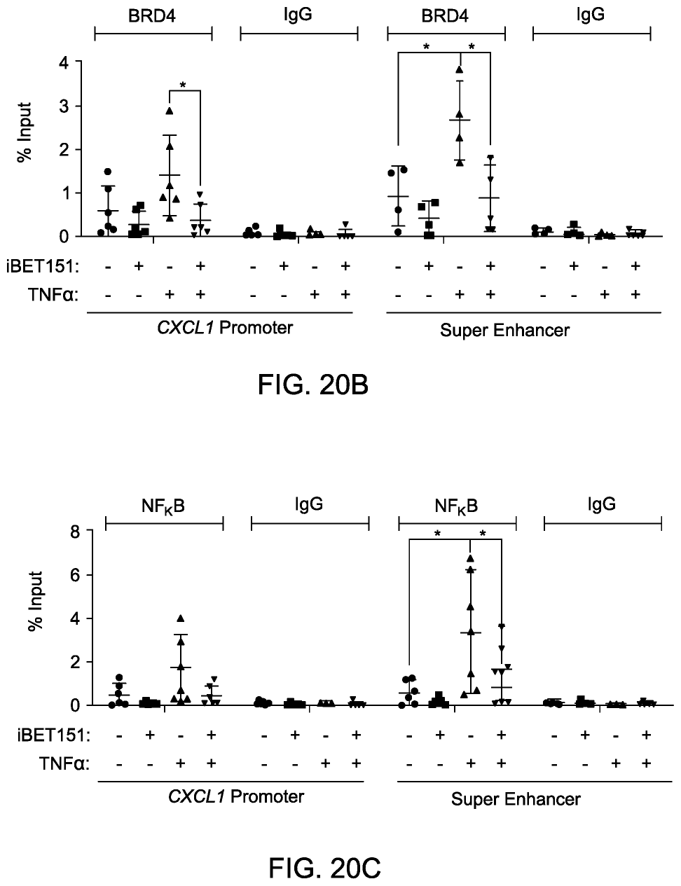

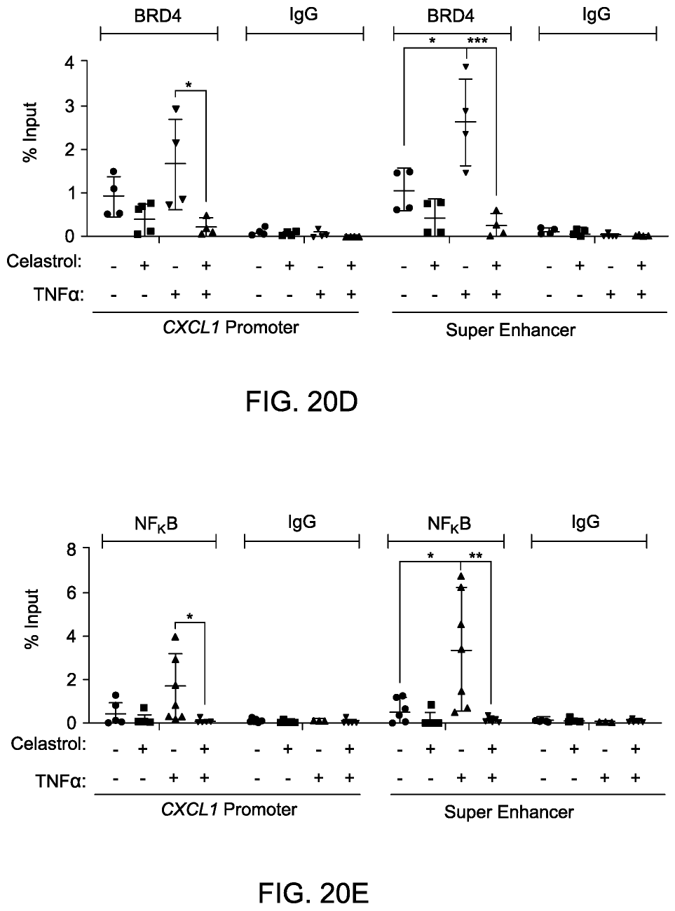

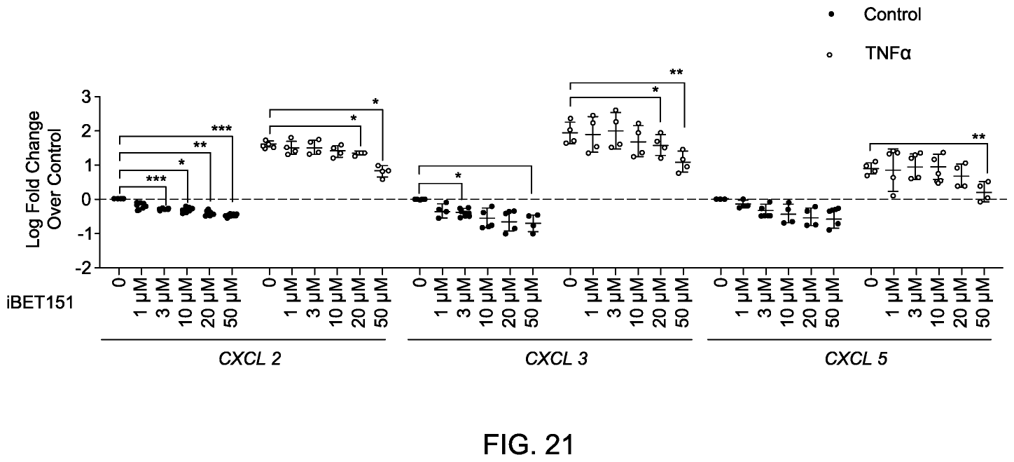

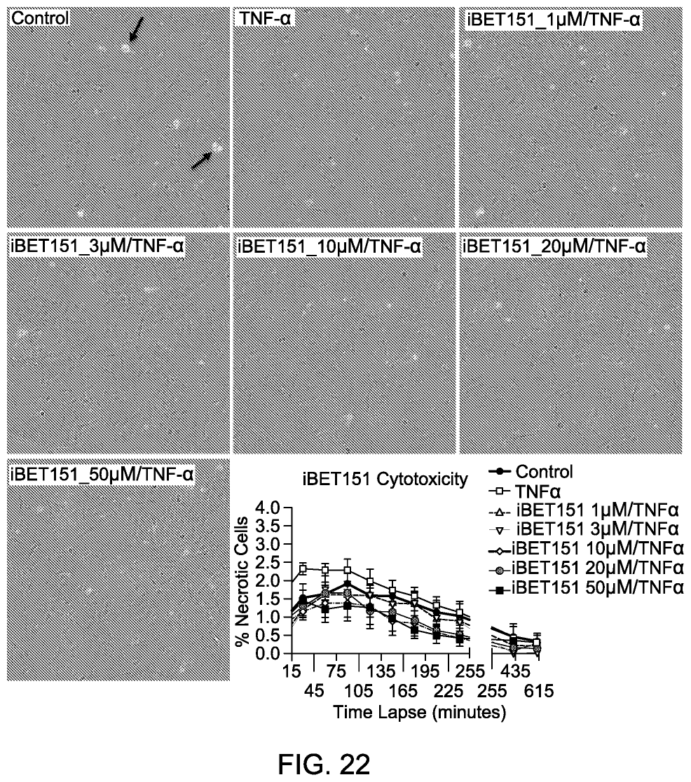

The transcription regulator and epigenetic reader BRD4 contributes to super enhancer function by maintaining super enhancer structure and facilitating the recruitment of other transcriptional cofactors. To observe the role of BRD4 in LSEC super enhancer function, iBET151, a commercially available pan-BET inhibitor that has highly specific activity against both bromodomains (BD) of all four BET proteins (BRD2, 3, 4 and T; see, e.g., Seal et al., Bioorg. Med. Chem. Lett., 22(8):2968-72 (2012)), was studied. It was found that LSEC CXCL expression was significantly decreased in the presence of iBET151 in vitro (FIG. 20A, FIG. 21A) without significant cytotoxicity (FIG. 22). The inhibitory effect of iBET151 on BRD4 occupancy at both the CXCL super enhancer and CXCL1 promoter sites was confirmed by ChIP (FIG. 20B). There was also a trend for decreased NF-κB binding at these sites after iBET151 treatment (FIG. 20C). A similar pattern was seen with celastrol, which restricts NF-κB nuclear translation, leading to decreased binding of both NF-κB and BRD4 (FIG. 20D, 20E).

### BET Inhibition Reduces Cxcl Expression and Neutrophilic Infiltration in Murine Models of AH

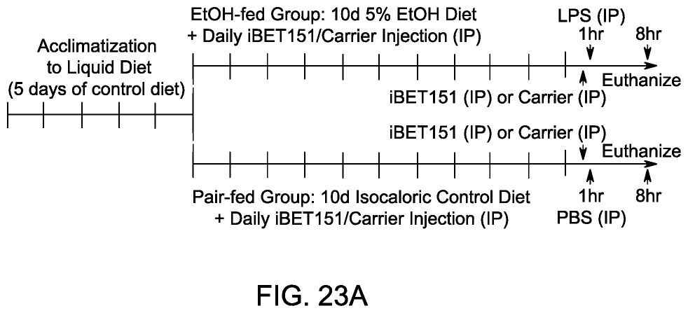

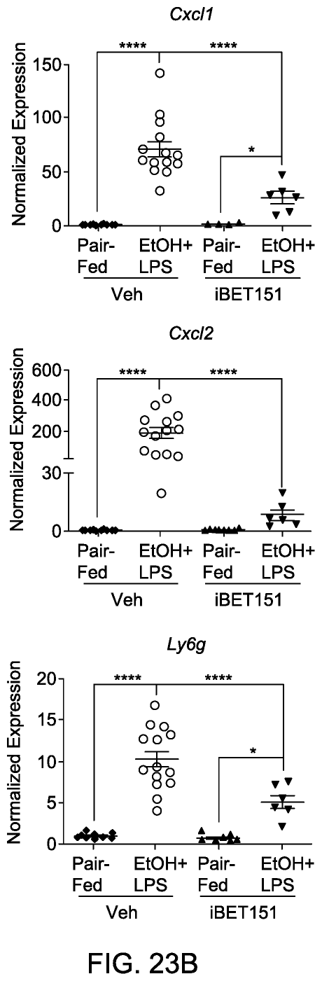

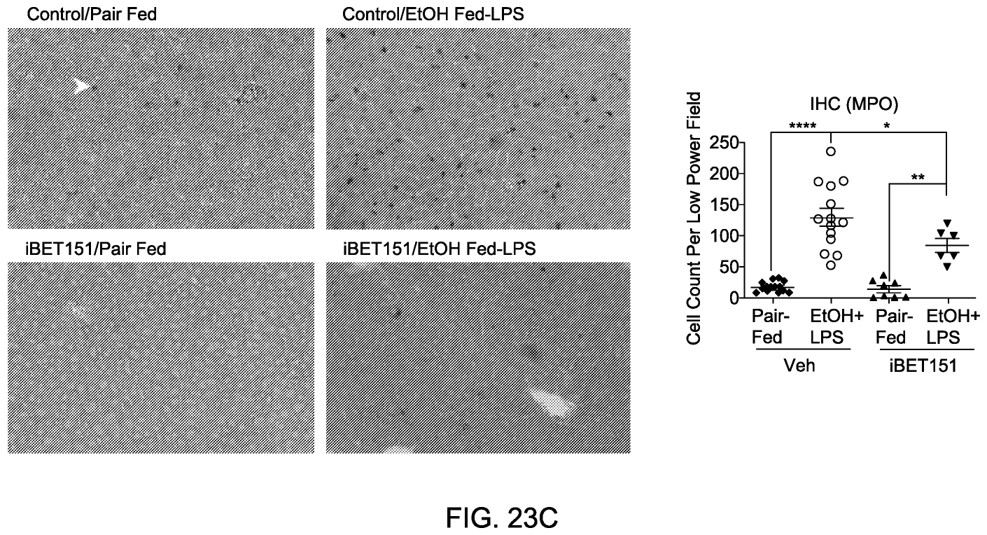

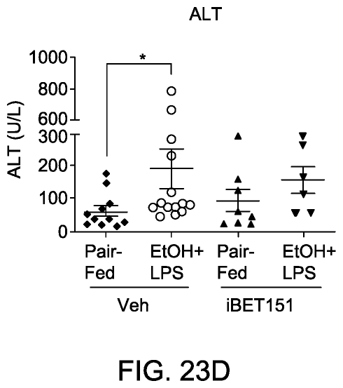

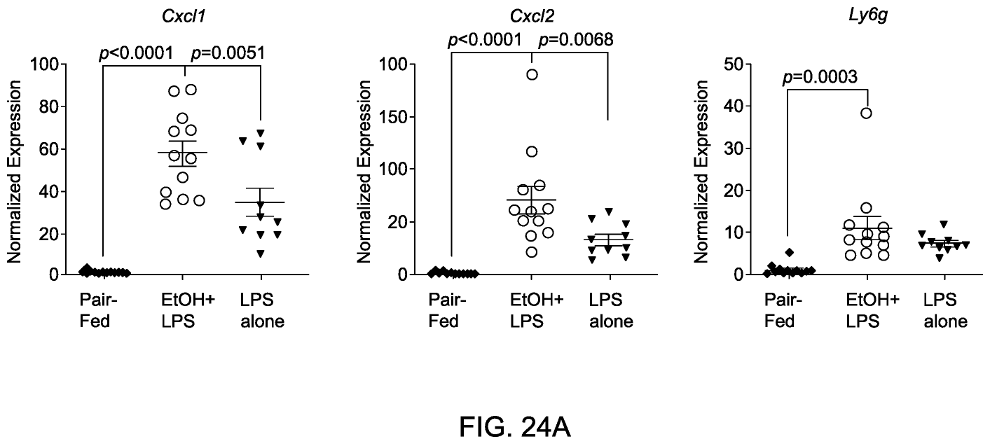

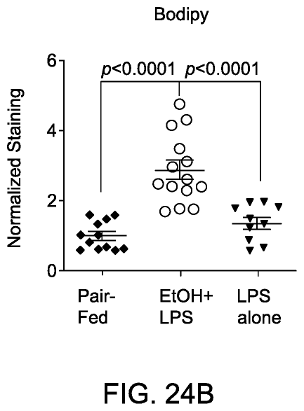

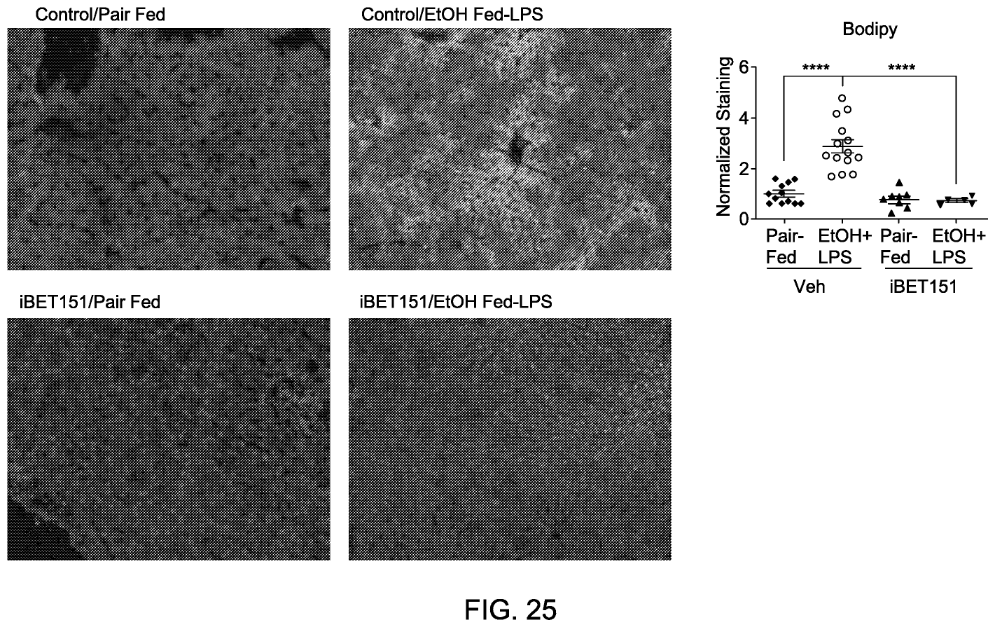

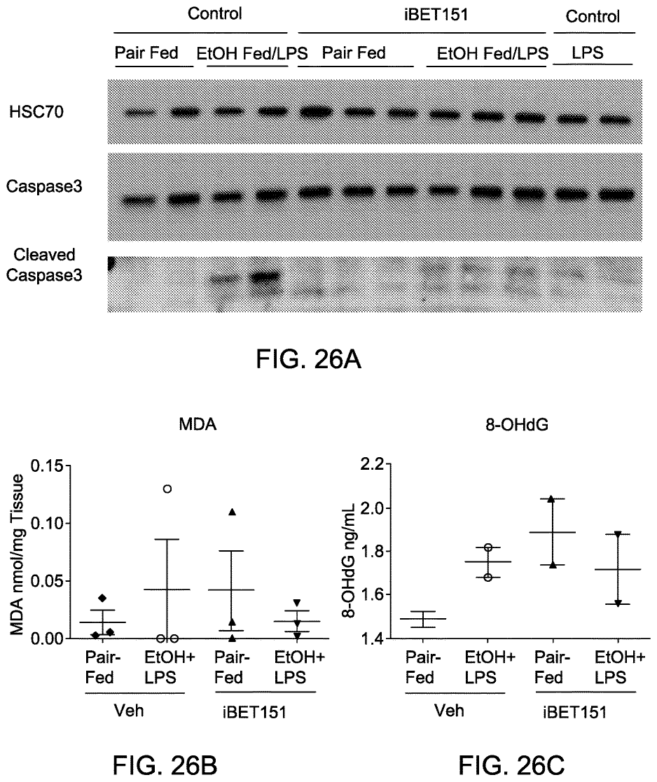

To generate an in vivo model with more exaggerated inflammation that could simulate human AH, a protocol of the chronic alcohol feeding model was modified by substituting the alcohol binge with a single LPS injection (FIG. 23A; see, also, Kong et al., Sci. Rep., 7(1):9292 (2017)). Dramatically elevated hepatic expression of Cxcl1 and Cxcl2 was observed in mice that underwent chronic alcohol feeding and LPS injection (FIG. 23B). Compared to mice given LPS injection alone, alcohol fed/LPS mice showed increased Cxcl1 and Cxcl2 expression and worsened steatosis (FIG. 24). In alcohol fed/LPS mice compared to pair-fed mice, there is increased neutrophil infiltration of the liver demonstrated by higher Ly6g expression (FIG. 23B) and increased IHC staining for MPO (FIG. 23C). Increased steatosis by BODIPY stain (FIG. 25) was observed in alcohol fed/LPS mice. There was a significant increase in serum alanine aminotransferase (ALT) in alcohol fed/LPS mice (FIG. 23D). To assess the role of super enhancer activation in alcohol fed/LPS induced liver inflammation, the pan-BET inhibitor iBET151 was given to mice as daily IP injections alongside alcohol feeding or LPS injection. Expression of Cxcl1 and Cxcl2 was significantly decreased in mice treated with iBET151 (FIG. 23B). iBET151 administration concurrently decreased neutrophil infiltration and steatosis (FIGS. 23B and 23C) but did not show a statistically significant decrease in ALT levels in alcohol fed/LPS mice. There was also a decrease in cleaved caspase 3 level in alcohol fed/LPS mice given iBET151, suggesting decreased apoptosis pathway activation with iBET151 administration (FIG. 26). These in vivo data suggest that suppression of super enhancer activity by pharmacologic BET inhibitor decreases hepatic expression of Cxcl chemokines which reduces liver inflammation in a murine model of human AH, consistent with the findings in LSECs in vitro.

### Example 6: Exemplary Embodiments

Embodiment 1. A method for treating a mammal having an alcohol-associated liver disease (ALD), wherein said method comprises:
identifying said mammal as having greater than 1.5×10¹¹ circulating extracellular vesicles (EVs) per milliliter (mL) in a sample obtained from said mammal; and
administering an IL-22 polypeptide or an ALD treatment to said mammal.

Embodiment 2. A method for treating a mammal having an ALD, wherein said method comprises administering an IL-22 polypeptide or an ALD treatment to a mammal identified as having greater than 1.5×10¹¹ circulating EVs per mL in a sample obtained from said mammal.

Embodiment 3. The method of Embodiment 1 or Embodiment 2, wherein said mammal is a human.

Embodiment 4. The method of any one of Embodiments 1-3, wherein said sample is a blood sample.

Embodiment 5. The method of Embodiment 4, wherein said blood sample is a plasma sample.

Embodiment 6. The method of any one of Embodiments 1-5, wherein said ALD is alcoholic hepatitis.

Embodiment 7. The method of any one of Embodiments 1-6, wherein said circulating EVs comprise an enriched sphingolipid cargo.

Embodiment 8. The method of Embodiment 7, wherein said enriched sphingolipid cargo is selected from the group consisting of sphinganine (SPA), sphingosine (SPH), sphingosine-1-phosphate (S1P), a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 9. The method of Embodiment 8, wherein said enriched sphingolipid cargo is said SPA, and wherein said circulating EVs comprise from about 0.9 nM to about 34.02 nM of said SPA.

Embodiment 10. The method of Embodiment 8, wherein said enriched sphingolipid cargo is said 14:0 ceramide, and wherein said circulating EVs comprise from about 0.25 nM to about 54.7 nM of said 14:0 ceramide.

Embodiment 11. The method of Embodiment 8, wherein said enriched sphingolipid cargo is said C16:0 ceramide, and wherein said circulating EVs comprise from about 19 nM to about 1765.9 nM of said C16:0 ceramide.

Embodiment 12. The method of Embodiment 8, wherein said enriched sphingolipid cargo is said C24:0 ceramide, and wherein said circulating EVs comprise from about 23.2 nM to about 2030.62 nM of said C24:0 ceramide.

Embodiment 13. The method of any one of Embodiments 1-12, said method comprising administering said ALD treatment to said mammal, wherein said ALD treatment is selected from the group consisting of administering nutritional supplementation, alcohol cessation counseling, administering corticosteroids, and administering pentoxifylline.

Embodiment 14. A method for treating a mammal having an ALD and as being at high risk of mortality, wherein said method comprises:
identifying said mammal as having greater than 5×10¹¹ circulating EVs per mL in a sample obtained from said mammal; and
administering an IL-22 polypeptide or an ALD treatment to said mammal.

Embodiment 15. A method for treating a mammal having an ALD and as being at high risk of mortality, wherein said method comprises administering an IL-22 polypeptide or an ALD treatment to a mammal identified as having greater than 5×10¹¹ circulating EVs per mL in a sample obtained from said mammal.

Embodiment 16. The method of Embodiment 14 or Embodiment 15, wherein said mammal is a human.

Embodiment 17. The method of any one of Embodiments 14-16, wherein said sample is a blood sample.

Embodiment 18. The method of Embodiment 17, wherein said blood sample is a plasma sample.

Embodiment 19. The method of any one of Embodiments 14-18, wherein said ALD is alcoholic hepatitis.

Embodiment 20. The method of any one of Embodiments 14-19, wherein said circulating EVs comprise an enriched sphingolipid cargo.

Embodiment 21. The method of Embodiment 20, wherein said enriched sphingolipid cargo is selected from the group consisting of SPA, SPH, S1P, a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 22. The method of Embodiment 21, wherein said enriched sphingolipid cargo is said SPA, and wherein said circulating EVs comprise from about 0.9 nM to about 34.02 nM of said SPA.

Embodiment 23. The method of Embodiment 21, wherein said enriched sphingolipid cargo is said 14:0 ceramide, and wherein said circulating EVs comprise from about 0.25 nM to about 54.7 nM of said 14:0 ceramide.

Embodiment 24. The method of Embodiment 21, wherein said enriched sphingolipid cargo is said C16:0 ceramide, and wherein said circulating EVs comprise from about 19 nM to about 1765.9 nM of said C16:0 ceramide.

Embodiment 25. The method of Embodiment 21, wherein said enriched sphingolipid cargo is said C24:0 ceramide, and wherein said circulating EVs comprise from about 23.2 nM to about 2030.62 nM of said C24:0 ceramide.

Embodiment 26. The method of any one of Embodiments 14-25, said method comprising administering said ALD treatment to said mammal, wherein said ALD treatment is selected from the group consisting of administering corticosteroids, administering pentoxifylline, and/or undergoing a liver transplantation.

Embodiment 27. A method for treating a mammal having an alcohol-associated liver disease (ALD), wherein said method comprises:
identifying said mammal as having circulating extracellular vesicles (EVs) comprising enriched sphingolipid cargo in a sample obtained from said mammal; and
administering an IL-22 polypeptide or an ALD treatment to said mammal.

Embodiment 28. A method for treating a mammal having an ALD, wherein said method comprises administering an IL-22 polypeptide or an ALD treatment to a mammal identified as having circulating EVs comprising enriched sphingolipid cargo in a sample obtained from said mammal.

Embodiment 29. The method of Embodiment 27 or Embodiment 28, wherein said mammal is a human.

Embodiment 30. The method of any one of Embodiments 27-29, wherein said sample is a blood sample.

Embodiment 31. The method of Embodiment 30, wherein said blood sample is a plasma sample.

Embodiment 32. The method of any one of Embodiments 27-31, wherein said ALD is alcoholic hepatitis.

Embodiment 33. The method of any one of Embodiments 27-32, wherein said enriched sphingolipid cargo is selected from the group consisting of SPA, SPH, S1P, a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 34. The method of Embodiment 33, wherein said enriched sphingolipid cargo is said SPA, and wherein said circulating EVs comprise from about 0.9 nM to about 34.02 nM of said SPA.

Embodiment 35. The method of Embodiment 33, wherein said enriched sphingolipid cargo is said 14:0 ceramide, and wherein said circulating EVs comprise from about 0.25 nM to about 54.7 nM of said 14:0 ceramide.

Embodiment 36. The method of Embodiment 33, wherein said enriched sphingolipid cargo is said C16:0 ceramide, and wherein said circulating EVs comprise from about 19 nM to about 1765.9 nM of said C16:0 ceramide.

Embodiment 37. The method of Embodiment 33, wherein said enriched sphingolipid cargo is said C24:0 ceramide, and wherein said circulating EVs comprise from about 23.2 nM to about 2030.62 nM of said C24:0 ceramide.

Embodiment 38. The method of any one of Embodiments 27-37, said method comprising administering said ALD treatment to said mammal, wherein said ALD treatment is selected from the group consisting of administering nutritional supplementation, alcohol cessation counseling, administering corticosteroids, and administering pentoxifylline.

Embodiment 39. A method for identifying a mammal having an alcohol-associated liver disease (ALD), wherein said method comprises:
detecting the presence of greater than 1.5×10¹¹ circulating extracellular vesicles (EVs) per milliliter (mL) in a sample obtained from said mammal; and
classifying said mammal as having said ALD.

Embodiment 40. The method of Embodiment 39, wherein said mammal is a human.

Embodiment 41. The method of any one of Embodiment 39 or Embodiment 40, wherein said sample is a blood sample.

Embodiment 42. The method of Embodiment 41, wherein said blood sample is a plasma sample.

Embodiment 43. The method of any one of Embodiments 39-42, wherein said ALD is alcoholic hepatitis.

Embodiment 44. The method of any one of Embodiments 39-43, wherein said circulating EVs comprise an enriched sphingolipid cargo.

Embodiment 45. The method of Embodiment 44, wherein said enriched sphingolipid cargo is selected from the group consisting of SPA, SPH, S1P, a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 46. The method of Embodiment 45, wherein said enriched sphingolipid cargo is said SPA, and wherein said circulating EVs comprise from about 0.9 nM to about 34.02 nM of said SPA.

Embodiment 47. The method of Embodiment 45, wherein said enriched sphingolipid cargo is said 14:0 ceramide, and wherein said circulating EVs comprise from about 0.25 nM to about 54.7 nM of said 14:0 ceramide.

Embodiment 48. The method of Embodiment 45, wherein said enriched sphingolipid cargo is said C16:0 ceramide, and wherein said circulating EVs comprise from about 19 nM to about 1765.9 nM of said C16:0 ceramide.

Embodiment 49. The method of Embodiment 45, wherein said enriched sphingolipid cargo is said C24:0 ceramide, and wherein said circulating EVs comprise from about 23.2 nM to about 2030.62 nM of said C24:0 ceramide.

Embodiment 50. A method for identifying a mammal having an ALD and as being at high risk of mortality, wherein said method comprises:
detecting the presence of greater than 5×10¹¹ circulating EVs per mL in a sample obtained from said mammal; and
classifying said mammal as having said ALD and as being at high risk of mortality.

Embodiment 51. The method of Embodiment 50, wherein said mammal is a human.

Embodiment 52. The method of any one of Embodiment 50 or Embodiment 51, wherein said sample is a blood sample.

Embodiment 53. The method of Embodiment 52, wherein said blood sample is a plasma sample.

Embodiment 54. The method of any one of Embodiments 50-53, wherein said ALD is alcoholic hepatitis.

Embodiment 55. The method of any one of Embodiments 50-54, wherein said circulating EVs comprise an enriched sphingolipid cargo.

Embodiment 56. The method of Embodiment 55, wherein said enriched sphingolipid cargo is selected from the group consisting of SPA, SPH, S1P, a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 57. The method of Embodiment 56, wherein said enriched sphingolipid cargo is said SPA, and wherein said circulating EVs comprise from about 0.9 nM to about 34.02 nM of said SPA.

Embodiment 58. The method of Embodiment 56, wherein said enriched sphingolipid cargo is said 14:0 ceramide, and wherein said circulating EVs comprise from about 0.25 nM to about 54.7 nM of said 14:0 ceramide.

Embodiment 59. The method of Embodiment 56, wherein said enriched sphingolipid cargo is said C16:0 ceramide, and wherein said circulating EVs comprise from about 19 nM to about 1765.9 nM of said C16:0 ceramide.

Embodiment 60. The method of Embodiment 56, wherein said enriched sphingolipid cargo is said C24:0 ceramide, and wherein said circulating EVs comprise from about 23.2 nM to about 2030.62 nM of said C24:0 ceramide.

Embodiment 61. A method for identifying a mammal having an alcohol-associated liver disease (ALD), wherein said method comprises:
detecting the presence of circulating extracellular vesicles (EVs) comprising enriched sphingolipid cargo in a sample obtained from said mammal; and
classifying said mammal as having said ALD.

Embodiment 62. The method of Embodiment 61, wherein said mammal is a human.

Embodiment 63. The method of Embodiment 61 or Embodiment 62, wherein said sample is a blood sample.

Embodiment 64. The method of Embodiment 63, wherein said blood sample is a plasma sample.

Embodiment 65. The method of any one of Embodiments 61-64, wherein said ALD is alcoholic hepatitis.

Embodiment 66. The method of any one of Embodiments 61-65, wherein said enriched sphingolipid cargo is selected from the group consisting of SPA, SPH, S1P, a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 67. The method of Embodiment 66, wherein said enriched sphingolipid cargo is said SPA, and wherein said circulating EVs comprise from about 0.9 nM to about 34.02 nM of said SPA.

Embodiment 68. The method of Embodiment 66, wherein said enriched sphingolipid cargo is said 14:0 ceramide, and wherein said circulating EVs comprise from about 0.25 nM to about 54.7 nM of said 14:0 ceramide.

Embodiment 69. The method of Embodiment 66, wherein said enriched sphingolipid cargo is said C16:0 ceramide, and wherein said circulating EVs comprise from about 19 nM to about 1765.9 nM of said C16:0 ceramide.

Embodiment 70. The method of Embodiment 66, wherein said enriched sphingolipid cargo is said C24:0 ceramide, and wherein said circulating EVs comprise from about 23.2 nM to about 2030.62 nM of said C24:0 ceramide.

Embodiment 71. A method for treating a mammal having an alcohol-associated liver disease (ALD), wherein said method comprises:
identifying said mammal as having greater than 1.5×10¹¹ circulating extracellular vesicles (EVs) per milliliter (mL) in a sample obtained from said mammal; and
administering an inhibitor of a BRD4 polypeptide to said mammal.

Embodiment 72. A method for treating a mammal having an ALD, wherein said method comprises administering an inhibitor of a BRD4 polypeptide to a mammal identified as having greater than 1.5×10¹¹ circulating EVs per mL in a sample obtained from said mammal.

Embodiment 73. The method of Embodiment 71 or Embodiment 72, wherein said mammal is a human.

Embodiment 74. The method of any one of Embodiments 71-73, wherein said sample is a blood sample.

Embodiment 75. The method of Embodiment 74, wherein said blood sample is a plasma sample.

Embodiment 76. The method of any one of Embodiments 71-75, wherein said ALD is alcoholic hepatitis.

Embodiment 77. The method of any one of Embodiments 71-76, wherein 30 said circulating EVs comprise an enriched sphingolipid cargo.

Embodiment 78. The method of Embodiment 77, wherein said enriched sphingolipid cargo is selected from the group consisting of sphinganine (SPA), sphingosine (SPH), sphingosine-1-phosphate (S1P), a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 79. The method of Embodiment 78, wherein said enriched sphingolipid cargo is said SPA, and wherein said circulating EVs comprise from about 0.9 nM to about 34.02 nM of said SPA.

Embodiment 80. The method of Embodiment 78, wherein said enriched sphingolipid cargo is said 14:0 ceramide, and wherein said circulating EVs comprise from about 0.25 nM to about 54.7 nM of said 14:0 ceramide.

Embodiment 81. The method of Embodiment 78, wherein said enriched sphingolipid cargo is said C16:0 ceramide, and wherein said circulating EVs comprise from about 19 nM to about 1765.9 nM of said C16:0 ceramide.

Embodiment 82. The method of Embodiment 78, wherein said enriched sphingolipid cargo is said C24:0 ceramide, and wherein said circulating EVs comprise from about 23.2 nM to about 2030.62 nM of said C24:0 ceramide.

Embodiment 83. The method of any one of Embodiments 71-82, wherein said inhibitor is iBET151, RVX-208, iBET 762, Zen-3694, JQ1, OTX-015, GSK620, or ABBV-744.

Embodiment 84. A method for treating a mammal having an ALD and as being at high risk of mortality, wherein said method comprises:
identifying said mammal as having greater than 5×10¹¹ circulating EVs per mL in a sample obtained from said mammal; and
administering an inhibitor of a BRD4 polypeptide to said mammal.

Embodiment 85. A method for treating a mammal having an ALD and as being at high risk of mortality, wherein said method comprises administering an inhibitor of a BRD4 polypeptide to a mammal identified as having greater than 5×10¹¹ circulating EVs per mL in a sample obtained from said mammal.

Embodiment 86. The method of Embodiment 84 or Embodiment 85, wherein said mammal is a human.

Embodiment 87. The method of any one of Embodiments 84-86, wherein said sample is a blood sample.

Embodiment 88. The method of Embodiment 87, wherein said blood sample is a plasma sample.

Embodiment 89. The method of any one of Embodiments 84-88, wherein said ALD is alcoholic hepatitis.

Embodiment 90. The method of any one of Embodiments 84-89, wherein said circulating EVs comprise an enriched sphingolipid cargo.

Embodiment 91. The method of Embodiment 90, wherein said enriched sphingolipid cargo is selected from the group consisting of SPA, SPH, S1P, a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 92. The method of Embodiment 91, wherein said enriched sphingolipid cargo is said SPA, and wherein said circulating EVs comprise from about 0.9 nM to about 34.02 nM of said SPA.

Embodiment 93. The method of Embodiment 91, wherein said enriched sphingolipid cargo is said 14:0 ceramide, and wherein said circulating EVs comprise from about 0.25 nM to about 54.7 nM of said 14:0 ceramide.

Embodiment 94. The method of Embodiment 91, wherein said enriched sphingolipid cargo is said C16:0 ceramide, and wherein said circulating EVs comprise from about 19 nM to about 1765.9 nM of said C16:0 ceramide.

Embodiment 95. The method of Embodiment 91, wherein said enriched sphingolipid cargo is said C24:0 ceramide, and wherein said circulating EVs comprise from about 23.2 nM to about 2030.62 nM of said C24:0 ceramide.

Embodiment 96. The method of any one of Embodiments 84-95, wherein said inhibitor is iBET151, RVX-208, iBET 762, Zen-3694, JQ1, OTX-015, GSK620, or ABBV-744.

Embodiment 97. A method for treating a mammal having an alcohol-associated liver disease (ALD), wherein said method comprises:
identifying said mammal as having circulating extracellular vesicles (EVs) comprising enriched sphingolipid cargo in a sample obtained from said mammal; and
administering an inhibitor of a BRD4 polypeptide to said mammal.

Embodiment 98. A method for treating a mammal having an ALD, wherein said method comprises administering an inhibitor of a BRD4 polypeptide to a mammal identified as having circulating EVs comprising enriched sphingolipid cargo in a sample obtained from said mammal.

Embodiment 99. The method of Embodiment 97 or Embodiment 98, wherein said mammal is a human.

Embodiment 100. The method of any one of Embodiments 97-99, wherein said sample is a blood sample.

Embodiment 101. The method of Embodiment 100, wherein said blood sample is a plasma sample.

Embodiment 102. The method of any one of Embodiments 97-101, wherein said ALD is alcoholic hepatitis.

Embodiment 103. The method of any one of Embodiments 97-102, wherein said enriched sphingolipid cargo is selected from the group consisting of SPA, SPH, S1P, a 14:0 ceramide, a C16:0 ceramide, a C18:0 ceramide, a C20:0 ceramide, a C22:0 ceramide, a C24:1 ceramide, a C24:0 ceramide, and combinations thereof.

Embodiment 104. The method of Embodiment 103, wherein said enriched sphingolipid cargo is said SPA, and wherein said circulating EVs comprise from about 0.9 nM to about 34.02 nM of said SPA.

Embodiment 105. The method of Embodiment 103, wherein said enriched sphingolipid cargo is said 14:0 ceramide, and wherein said circulating EVs comprise from about 0.25 nM to about 54.7 nM of said 14:0 ceramide.

Embodiment 106. The method of Embodiment 103, wherein said enriched sphingolipid cargo is said C16:0 ceramide, and wherein said circulating EVs comprise from about 19 nM to about 1765.9 nM of said C16:0 ceramide.

Embodiment 107. The method of Embodiment 33, wherein said enriched sphingolipid cargo is said C24:0 ceramide, and wherein said circulating EVs comprise from about 23.2 nM to about 2030.62 nM of said C24:0 ceramide.

Embodiment 108. The method of any one of Embodiments 97-107, wherein said inhibitor is iBET151, RVX-208, iBET 762, Zen-3694, JQ1, OTX-015, GSK620, or ABBV-744.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having an alcohol-associated liver disease (ALD), wherein said method comprises:
identifying said mammal as having greater than 1.5×10¹¹ circulating extracellular vesicles (EVs) per milliliter (mL) in a sample obtained from said mammal; and
administering an inhibitor of a BRD4 polypeptide to said mammal.

2. (canceled)

3. The method of claim 1, wherein said mammal is a human.

4. The method of claim 1, wherein said sample is a blood sample.

5. (canceled)

6. The method of claim 1, wherein said ALD is alcoholic hepatitis.

7-12. (canceled)

13. The method of claim 1, wherein said inhibitor is iBET151, RVX-208, iBET 762, Zen-3694, JQ1, OTX-015, GSK620, or ABBV-744.

14. A method for treating a mammal having an ALD and as being at high risk of mortality, wherein said method comprises:
identifying said mammal as having greater than 5×10¹¹ circulating EVs per mL in a sample obtained from said mammal; and
administering an inhibitor of a BRD4 polypeptide to said mammal.

15-26. (canceled)

27. A method for treating a mammal having an alcohol-associated liver disease (ALD), wherein said method comprises:
identifying said mammal as having circulating extracellular vesicles (EVs) comprising enriched sphingolipid cargo in a sample obtained from said mammal; and
administering an inhibitor of a BRD4 polypeptide to said mammal.

28. (canceled)

29. The method of claim 27, wherein said mammal is a human.

30. The method of claim 27, wherein said sample is a blood sample.

31. (canceled)

32. The method of claim 27, wherein said ALD is alcoholic hepatitis.

33. The method of claim 27, wherein said enriched sphingolipid cargo is selected from the group consisting of SPA, SPH, S1P, a 14:0 ceramide, a C16:0 ceramide, a C18:0 ceramide, a C20:0 ceramide, a C22:0 ceramide, a C24:1 ceramide, a C24:0 ceramide, and combinations thereof.

34. The method of claim 33, wherein said enriched sphingolipid cargo is said SPA, and wherein said circulating EVs comprise from about 0.9 nM to about 34.02 nM of said SPA.

35. The method of claim 33, wherein said enriched sphingolipid cargo is said 14:0 ceramide, and wherein said circulating EVs comprise from about 0.25 nM to about 54.7 nM of said 14:0 ceramide.

36. The method of claim 33, wherein said enriched sphingolipid cargo is said C16:0 ceramide, and wherein said circulating EVs comprise from about 19 nM to about 1765.9 nM of said C16:0 ceramide.

37. The method of claim 33, wherein said enriched sphingolipid cargo is said C24:0 ceramide, and wherein said circulating EVs comprise from about 23.2 nM to about 2030.62 nM of said C24:0 ceramide.

38. The method of claim 27, wherein said inhibitor is iBET151, RVX-208, iBET 762, Zen-3694, JQ1, OTX-015, GSK620, or ABBV-744.

39. A method for treating a mammal having an alcohol-associated liver disease (ALD), wherein said method comprises:
identifying said mammal as having greater than 1.5×10¹¹ circulating extracellular vesicles (EVs) per milliliter (mL) in a sample obtained from said mammal; and
administering an IL-22 polypeptide or an ALD treatment to said mammal.

40. (canceled)

41. The method of claim 39, wherein said mammal is a human.

42. The method of claim 39, wherein said sample is a blood sample.

43. (canceled)

44. The method of claim 39, wherein said ALD is alcoholic hepatitis.

45-50. (canceled)

51. The method of claim 39, said method comprising administering said ALD treatment to said mammal, wherein said ALD treatment is selected from the group consisting of administering nutritional supplementation, alcohol cessation counseling, administering corticosteroids, and administering pentoxifylline.
